(19) United States
(12) Patent Application Publication
(10) Pub. No.: US 2005/0054682 A1
(43) Pub. Date: Mar. 10, 2005

(54) Pharmaceutical compositions comprising substituted benzimidazoles and methods of using same

(76) Inventor: Jeffrey O. Phillips, Ashland, MO (US)

Correspondence Address: MAYER, BROWN, ROWE & MAW LLP, 190 SOUTH LASALLE ST, CHICAGO, IL, 60603-3441, US

(21) Appl. No.: 10/898,135

(22) Filed: Jul. 23, 2004

Related U.S. Application Data
(63) Continuation-in-part of application No. 10/722,184, filed on Nov. 25, 2003.
Application No. 10/722,184 is a continuation of application No. 10/054,350, filed on Jan. 19, 2002, now Pat. No. 6,699,885.
Application No. 10/054,350 is a continuation-in-part of application No. 09/901,942, filed on Jul. 9, 2001, now Pat. No. 6,645,988.
Application No. 09/901,942 is a continuation-in-part of application No. 09/481,207, filed on Jan. 11, 2000, now Pat. No. 6,489,346.
Application No. 09/481,207 is a continuation-in-part of application No. 09/183,422, filed on Oct. 30, 1998, now abandoned.
Application No. 09/183,422 is a continuation-in-part of application No. 08/680,376, filed on Jul. 15, 1996, now Pat. No. 5,840,737.
(60) Provisional application No. 60/009,608, filed on Jan. 4, 1996.

(51) Int. Cl. A61K 31/4439

(52) U.S. Cl. 514/338

(57) ABSTRACT

The present invention is directed to, inter alia, pharmaceutical compositions comprising at least one proton pump inhibitor and at least one buffering agent. Compositions of the invention are useful in treating, inter alia, gastric acid related disorders.

This application is a continuation-in-part of co-pending U.S. patent application Ser. No. 10/722,184, filed Nov. 25, 2003, which is a continuation of U.S. patent application Ser. No. 10/054,350, filed Jan. 19, 2002, which is a continuation-in-part of U.S. patent application Ser. No. 09/901,942, now U.S. Pat. No. 6,645,988, filed Jul. 9, 2001, which is a continuation-in-part of U.S. patent application Ser. No. 09/481,207, filed Jan. 11, 2000, now U.S. Pat. No. 6,489,346, which is a continuation-in-part of U.S. patent application Ser. No. 09/183,422, filed Oct. 29, 1998, now abandoned, which is a continuation-in-part of U.S. patent application Ser. No. 08/680,376, filed Jul. 15, 1996, now U.S. Pat. No. 5,840,737, which claimed priority of U.S. Provisional Application Ser. No. 60/009,608, filed Jan. 4, 1996. This application claims priority to all such previous applications, and all such applications are hereby incorporated by reference herein in their entirety.

### BACKGROUND OF THE INVENTION

Gastrointestinal disorders such as active duodenal ulcers, gastric ulcers, gastroesophageal reflux disease (GERD), severe erosive esophagitis, poorly responsive symptomatic GERD, and pathological hypersecretory conditions such as Zollinger Ellison syndrome represent a major health concern impacting millions of people globally. In fact, it is estimated that as many as 60 million Americans alone experience acid reflux at least once a month, while approximately 19 million Americans suffer from GERD.

In the past, the above-described (and other related) gastrointestinal disorders and their associated symptoms have been treated with H₂ histamine antagonists and antacids. Unfortunately, many such available treatments are not completely effective in ameliorating the disorders themselves or their symptoms; additionally, many produce adverse side effects including, among others, constipation, diarrhea, and thrombocytopenia. Moreover, H₂ antagoinists such as ranitidine and cimetidine are relatively costly modes of therapy.

More recently, at least some of the above-described gastrointestinal disorders have been treated with proton pump inhibitors (also called PPIs). PPIs are believed to reduce gastric acid production by inhibiting H⁺, K⁺-ATPase of the parietal cel—the final common pathway for gastric acid secretion. One particular class of PPIs includes substituted benzimidazole compounds that contain a sulfinyl group bridging substituted benzimidazole and pyridine rings.

At neutral pH, these PPIs are chemically stable, lipid-soluble compounds that have little or no inhibitory activity. It is believed that the neutral PPIs reach parietal cells from the blood and diffuse into the secretory canaliculi where they become protonated and thereby trapped. The protonated agent is then believed to rearrange to form a sulfenic acid and a sulfenamide. The sulfenamide, in turn, is thought to interact covalently with sulfhydryl groups at critical sites in the extracellular (luminal) domain of the membrane-spanning H⁺, K⁺-ATPase. See, Hardman et al., Goodman & Gilman's The Pharmacological Basis of Therapeutics, p. 907, 9^{th} ed. (1996).

Unfortunately, commercially available substituted benzimidazole compounds are unstable at neutral or acidic pH and undergo decomposition in gastrointestinal fluid upon oral administration, thereby resulting in loss of therapeutic activity. To overcome this acid instability, such compounds are typically formulated for oral delivery as enteric coated solid dosage forms, for example enteric coated tablets, which coating protects the drug from contact with acidic stomach secretions. An undesirable consequence of such enteric coating is that therapeutic onset time is significantly delayed by comparison with non-enteric coated dosage forms. Such prolonged time to therapeutic onset is particularly undesirable for patients in need of rapid relief from one or more of the above described disorders or symptoms.

For example, U.S. Pat. No. 4,786,505 to Lovgren et al. discloses that a pharmaceutical oral solid dosage form of omeprazole must be protected from contact with acidic gastric juice by an enteric coating to maintain its pharmaceutical activity. That patent describes an enteric coated omeprazole preparation containing an alkaline core comprising omeprazole, a subcoating over the core, and an enteric coating over the subcoating. Specific examples of enteric coated PPIs include omeprazole (Prilosec®), lansoprazole (Prevacid®), perprazole (also referred to as esomeprazole; Nexium®), rabeprazole (Aciphex®), and pantoprazole (Protonix®). Omeprazole, a substituted benzimidazole, has the following chemical name: 5-methoxy-2-[(4-methoxy-3,5-dimethyl-2-pyridinyl) methyl]sulfinyl]-1H-benzimidazole.

Due at least in part to the above problems and observations, it would be advantageous if pharmaceutical formulations comprising PPIs could be prepared that provide faster onset of therapeutic activity while overcoming or minimizing acid degradation problems.

### SUMMARY OF THE INVENTION

In one embodiment, the present invention provides an orally deliverable pharmaceutical composition comprising: (a) a therapeutically effective amount of at least one acid labile, substituted benzimidazole H⁺,K⁺-ATPase proton pump inhibitor; and (b) at least one buffering agent in a total amount greater than 10 mEq. In a related embodiment, the composition comprises substantially no or no poly[phosphoryl/sulfon]-ated carbohydrate and is in the form of a solid dosage unit. In still another related embodiment, if such a composition comprises a poly[phosphoryl/sulfon]-ated carbohydrate (e.g. sucralfate or sucrose octasulfate), the weight ratio of poly[phosphoryl/sulfon]-ated carbohydrate to buffering agent is less than 1:5 (0.2), less than 1:10 (0.1) or less than 1:20 (0.05). Alternatively, the poly[phosphoryl/sulfon]-ated carbohydrate is present in the composition, if at all, in an amount less than 50 mg, less than 25 mg, less than 10 mg or less than 5 mg.

In varying embodiments, compositions of the invention can be in the form of one or more orally deliverable dosage units. The terms “orally deliverable” or “oral administration” herein include any form of delivery of a therapeutic agent or a composition thereof to a subject wherein the agent or composition is placed in the mouth of the subject, whether or not the agent or composition is swallowed. Thus “oral administration” includes buccal and sublingual as well as esophageal administration.

Compositions provided by various embodiments of the present invention can be in the form of solids or liquids. In one embodiment, such compositions are in the form of discrete dosage units. The terms “dose unit” and/or “dosage unit” herein refer to a portion of a pharmaceutical composition that contains an amount of a therapeutic agent suitable for a single administration to provide a therapeutic effect.

Compositions of the invention are believed to provide, inter alia, one or more of: improved stability, decreased time to therapeutic effect, improved ease of handling, improved physical and/or chemical stability, more timely and/or consistent absorption, improved side-effect profile, reduced dosing amount and/or frequency, reduced time/cost to prepare, improved storage stability, and/or improved patient convenience and compliance.

Also provided herein are methods of treating gastrointestinal disorders by administering to a subject one or more compositions described herein. The phrase “gastrointestinal disorder” or “gastrointestinal disease” refers generally to a disorder or disease that occurs due an imbalance between acid and pepsin production on the one hand, so-called aggressive factors, and mucous, bicarbonate, and prostaglandin production on the other hand, so-called defensive factors. In mammals such disorders include, but are not limited to, duodenal ulcer, gastric ulcer, acid dyspepsia, gastroesophageal reflux disease (GERD), severe erosive esophagitis, poorly responsive symptomatic gastroesophageal reflux disease, heartburn, other esophageal disorders, and a gastrointestinal pathological hypersecretory condition such as Zollinger Ellison Syndrome.

### BRIEF DESCRIPTION OF THE DRAWINGS

FIG. 1 is a graph showing effect of omeprazole solution on gastric pH in patients at risk for upper gastrointestinal bleeding from stress-related mucosal damage.

FIG. 2 is a flow chart illustrating a patient enrollment scheme.

FIG. 3 is a bar graph illustrating gastric pH as measured both pre- and post-administration of omeprazole solution.

FIG. 4 is a graph illustrating stomach pH values after oral administration of both ChocoBase™ plus lansoprazole and lansoprazole alone.

FIG. 5 is a graph illustrating environmental pH values after administration of a proton pump inhibitor/buffering agent formulation.

FIG. 6 is a graph showing results of an in vitro stomach model experiment using sodium bicarbonate.

FIG. 7 is a graph showing results of an in vitro stomach model experiment using calcium carbonate.

### DETAILED DESCRIPTION OF THE INVENTION

Several specific embodiments of the present invention are discussed herein with the understanding that the present disclosure is to be considered only as an exemplification of the principles of the invention, and is not intended to limit the invention in any way. For example, where the present invention is illustrated herein with particular reference to omeprazole, lansoprazole, pantoprazole, rabeprazole, esomeprazole, pariprazole, or leminoprazole, it will be understood that any other proton pump inhibitor, if desired, can be substituted in whole or in part for the proton pump inhibitor described.

As described above, in one embodiment, the present invention provides an orally deliverable pharmaceutical composition comprising: (a) a therapeutically effective amount of at least one acid labile, substituted benzimidazole H⁺,K⁺-ATPase proton pump inhibitor; and (b) at least one buffering agent in a total amount greater than 10 mEq; the composition comprises substantially no or no amount of poly[phosphoryl/sulfon]-ated carbohydrate. The terms “substantially no amount of poly[phosphoryl/sulfon]-ated carbohydrate”, “substantially no poly[phosphoryl/sulfon]-ated carbohydrate”, “substantially no sucralfate”, “substantially no amount of sucralfate”, and related terms, in the present context, embrace situations in which no sucralfate or poly[phosphoryl/sulfon]-ated carbohydrate is present in a given composition and also embraces situations in which a de minimus amount of sucralfate or poly[phosphoryl/sulfon]-ated carbohydrate is present in a composition, for example to avoid a patent claim that excludes any amount of poly[phosphoryl/sulfon]-ated carbohydrate from being present.

In a related embodiment, if such a composition comprises a poly[phosphoryl/sulfon]-ated carbohydrate, the weight ratio of poly[phosphoryl/sulfon]-ated carbohydrate to buffering agent is less than 1:5 (0.2), less than 1:10 (0.1) or less than 1:20 (0.05). Alternatively, a poly[phosphoryl/sulfon]-ated carbohydrate is present in the composition, if at all, in an amount less than 50 mg, less than 25 mg, less than 10 mg or less than 5 mg.

In another embodiment, the invention provides an orally deliverable pharmaceutical composition comprising: (a) a therapeutically effective amount of at least one acid labile, substituted benzimidazole H⁺,K⁺-ATPase proton pump inhibitor; and (b) a combination of at least two non-amino acid buffering agents, wherein the combination of at least two non-amino acid buffering agents comprises substantially no aluminum hydroxide-sodium bicarbonate co-precipitate. In a related embodiment, the composition comprises substantially no or no poly[phosphoryl/sulfon]-ated carbohydrate and the composition is in the form of a solid dosage unit. In another related embodiment, if such a composition comprises a poly[phosphoryl/sulfon]-ated carbohydrate, the weight ratio of poly[phosphoryl/sulfon]-ated carbohydrate to buffering agent is less than 1:5 (0.2), less than 1:10 (0.1) or less than 1:20 (0.05). Alternatively, a poly[phosphoryl/sulfon]-ated carbohydrate is present in the composition, if at all, in an amount less than 50 mg, less than 25 mg, less than 10 mg or less than 5 mg.

In yet another embodiment, the present invention provides an orally deliverable pharmaceutical composition comprising: (a) a therapeutically effective amount of at least one acid labile, substituted benzimidazole H⁺,K⁺-ATPase proton pump inhibitor in a total amount of about 20 to about 40 mg; and (b) at least one non-amino acid buffering agent wherein the non-amino acid buffering agent is present in the composition in a total amount greater than 800 mg, the composition comprises substantially no or no amount of poly[phosphoryl/sulfon]-ated carbohydrate. In a related embodiment, if such a composition comprises a poly[phosphoryl/sulfon]-ated carbohydrate, the weight ratio of poly[phosphoryl/sulfon]-ated carbohydrate to buffering agent is less than 1:5 (0.2), less than 1:10 (0.1) or less than 1:20 (0.05). Alternatively, the poly[phosphoryl/sulfon]-ated carbohydrate is present in the composition, if at all, in an amount less than 50 mg, less than 25 mg, less than 10 mg or less than 5 mg.

In still another embodiment, the present invention provides an orally deliverable pharmaceutical composition comprising: (a) a therapeutically effective amount of at least one acid labile, substituted benzimidazole H⁺,K⁺-ATPase proton pump inhibitor; and (b) at least one buffering agent in a total amount greater than 10 mEq, wherein the composition comprises substantially no or no poly[phosphoryl/sulfon]-ated carbohydrate, and the composition is in the form of a solid dosage unit. In a related embodiment, if an amino acid buffering agent is present, at least one of the following conditions is met: (1) the weight ratio of amino acid buffering agent:proton pump inhibitor is greater than 20:1; (2) the composition comprises at least two non-amino acid buffering agents; (3) the composition comprises at least one non-amino acid buffering agent wherein the weight ratio of the at least one non-amino acid buffering agent:proton pump inhibitor is greater than 20:1; and/or (4) the weight ratio of total buffering agent:proton pump inhibitor is greater than 40:1. In another related embodiment, if such a composition comprises a poly[phosphoryl/sulfon]-ated carbohydrate, the weight ratio of poly[phosphoryl/sulfon]-ated carbohydrate to buffering agent is less than 1:5 (0.2), less than 1:10 (0.1) or less than 1:20 (0.05). Alternatively, a poly[phosphoryl/sulfon]-ated carbohydrate is present in the composition, if at all, in an amount less than 50 mg, less than 25 mg, less than 10 mg or less than 5 mg.

Proton Pump Inhibitors

Compositions of the invention comprise at least one pharmaceutically acceptable acid labile, substituted benzimidazole H⁺,K⁺-ATPase proton pump inhibitor (PPI). Illustrative PPIs are those compounds of Formula (I): 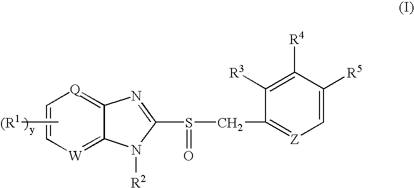
wherein
R¹ is hydrogen, alkyl, halogen, cyano, carboxy, carboalkoxy, carboalkoxyalkyl, carbamoyl, carbamoylalkyl, hydroxy, alkoxy which is optionally fluorinated, hydroxyalkyl, trifluoromethyl, acyl, carbamoyloxy, nitro, acyloxy, aryl, aryloxy, alkylthio, or alkylsulfinyl;
R² is hydrogen, alkyl, acyl, acyloxy, alkoxy, amino, aralkyl, carboalkoxy, carbamoyl, alkylcarbamoyl, dialkylcarbamoyl, alkylcarbonylmethyl, alkoxycarbonylmethyl, or alkylsulfonyl;
R³ and R⁵ are the same or different and each is hydrogen, alkyl, alkoxy, amino, or alkoxyalkoxy;
R⁴ is hydrogen, alkyl, alkoxy which may optionally be fluorinated, or alkoxyalkoxy;
Q is nitrogen, CH, or CR¹;
W is nitrogen, CH, or CR¹;
y is an integer of 0 through 4; and
Z is nitrogen, CH, or CR¹;
   or a free base, salt, ester, hydrate, amide, enantiomer, isomer, tautomer, prodrug, polymorph, or derivative thereof.

Specific examples of suitable PPIs include omeprazole, tenatoprazole, lansoprazole, rabeprazole, esomeprazole (also referred to as S-omeprazole), pantoprazole, pariprazole, leminoprazole and nepaprazole or a free base, a free acid, or a salt, hydrate, ester, amide, enantiomer, isomer, tautomer, polymorph, prodrug, or derivative of such compounds.

In another embodiment, compositions of the invention comprise at least one PPI in a total amount of about 1 mg to about 1000 mg, about 7.5 mg to about 750 mg, about 10 mg to about 500 mg, about 10 mg to about 100 mg, about 12.5 mg to about 250 mg, about 15 mg to about I 00 mg, or about 20 mg to about 40 mg.

Buffering Agent

Compositions of the invention comprise one or more pharmaceutically acceptable buffering agents. Buffering agents useful in the present invention include agents possessing pharmacological activity as a weak or strong base. In one embodiment, the buffering agent, when formulated with or administered substantially simultaneously with a PPI, functions to raise the pH of gastrointestinal fluid and thereby to substantially prevent or inhibit acid degradation of the PPI by gastrointestinal fluid.

In another embodiment, buffering agents useful in accordance with the present invention comprise a salt of a Group IA metal including, for example, a bicarbonate salt of a Group IA metal, a carbonate salt of a Group IA metal, an alkaline earth metal buffering agent, an amino acid, an alkaline salt of an amino acid, an aluminum buffering agent, a calcium buffering agent, a sodium buffering agent, or a magnesium buffering agent. Other suitable buffering agents include alkali (sodium and potassium) or alkaline earth (calcium and magnesium) carbonates, phosphates, bicarbonates, citrates, borates, acetates, phthalates, tartrates, succinates and the like.

Illustrative examples of specific buffering agents include, without limitation, aluminum hydroxide, aluminum hydroxide/magnesium carbonate, aluminum hydroxide/magnesium carbonate/calcium carbonate co-precipitate, aluminum magnesium hydroxide, aluminum hydroxide/magnesium hydroxide co-precipitate, aluminum hydroxide/sodium bicarbonate coprecipitate, aluminum glycinate, calcium acetate, calcium bicarbonate, calcium borate, calcium carbonate, calcium citrate, calcium chloride, calcium gluconate, calcium glycerophosphate, calcium hydroxide, calcium lactate, calcium phthalate, calcium phosphate, calcium succinate, calcium tartrate, dibasic sodium phosphate, dipotassium hydrogen phosphate, dipotassium phosphate, disodium hydrogen phosphate, disodium succinate, dry aluminum hydroxide gel, L-arginine, magnesium acetate, magnesium aluminate, magnesium borate, magnesium bicarbonate, magnesium carbonate, magnesium citrate, magnesium gluconate, magnesium hydroxide, magnesium lactate, magnesium metasilicate aluminate, magnesium oxide, magnesium phthalate, magnesium phosphate, magnesium silicate, magnesium succinate, magnesium tartrate, potassium acetate, potassium carbonate, potassium bicarbonate, potassium borate, potassium citrate, potassium metaphosphate, potassium phthalate, potassium phosphate, potassium polyphosphate, potassium pyrophosphate, potassium succinate, potassium tartrate, sodium acetate, sodium bicarbonate, sodium borate, sodium carbonate, sodium citrate, sodium gluconate, sodium dihydrogen phosphate, sodium hydrogen phosphate, sodium hydroxide, sodium lactate, sodium phthalate, sodium phosphate, sodium polyphosphate, sodium pyrophosphate, sodium sesquicarbonate, sodium succinate, sodium tartrate, sodium tripolyphosphate, synthetic hydrotalcite, tetrapotassium pyrophosphate, tetrasodium pyrophosphate, trihydroxymethylaminomethane, tripotassium phosphate, trisodium phosphate, and trometamol, and mixtures thereof. (Based in part upon the list provided in The Merck Index, Merck & Co. Rahway, N.J. (2001)). In addition, proteins or protein hydrolysates can serve as buffering agents owing to their ability to rapidly neutralize acid.

In another embodiment, one or more buffering agents are present in a composition of the invention in a total amount of about 0.05 mEq to about 10 mEq per mg of PPI, about 0.1 mEq to about 5 mEq per mg of PPI, or about 0.2 mEq to about 2.5 mEq per mg of PPI.

In another embodiment, one or more buffering agents are present in a total amount of about 0.5 mEq to about 150 mEq, about 1 mEq to about 150 mEq, about 10 mEq to about 150 mEq, about 10 mEq to about 75 mEq, about 10 mEq to about 60 mEq, or about 10 mEq to about 50 mEq.

In yet another embodiment, one or more buffering agents are present in a total amount of at least about 10 mEq, at least about 11 mEq, at least about 12 mEq, at least about 13 mEq, at least about 14 mEq, or at least about 15 mEq.

In still another embodiment, one or more buffering agents and the PPI are present in a weight ratio of at least about 5:1, at least about 7:1, at least about 10:1, at least about 20:1, greater than 20:1, at least about 21:1, at least about 22:1, at least about 23:1, at least about 25:1, at least about 30:1, at least about 35:1, at least about 40:1, greater than 40:1, or at least about 45:1.

In another embodiment, one or more buffering agents are present in a composition of the invention in a total amount of about 100 to about 20,000 mg, about 300 to about 10,000 mg, about 800 to about 2,500, about 800 to about 2,000 mg or about 800 to about 1,800 mg. Illustratively, the total amount of buffering agent present in a composition of this embodiment will be about 850 mg, about 900 mg, about 920 mg, 950 mg, about 1,000 mg, about 1,050 mg, about 1,100 mg, about 1,150 mg, about 1,200 mg, about 1,250 mg, about 1,300 mg, about 1,350 mg, about 1,400 mg, about 1,450 mg, about 1,500 mg, about 1,550 mg, about 1,600 mg, about 1,620 mg, about 1,640 mg, about 1,680 mg, about 1,700 mg, about 1,725 mg, about 1,750 mg, about 1,800 mg, about 1,825 mg, about 1,850 mg, about 1,875 mg, about 1,900 mg, about 1,950 mg, about 2,000 mg, or greater.

In another embodiment, one or more buffering agents are present in a composition of the invention in a total amount that is greater than 800 mg, for example at least about 920 mg or at least about 1,000 mg.

In still another embodiment, particularly where the composition is other than a dosage form selected from the group consisting of a suspension tablet, a chewable tablet, an effervescent powder, an effervescent tablet, lozenge and/or a troche, the buffering agent and PPI are present in a weight ratio greater than 20:1, not less than about 21:1, not less than about 22:1, not less than about 23:1, not less than about 24:1, not less than about 25:1, not less than about 26:1, not less than about 27:1, not less than about 28:, not less than about 29:, not less than about 30:1, not less than about 31:1, not less than about 32:1, not less than about 33:!, not less than about 34:1, not less than about 35:1, not less than about 36:1, not less than about 37:1, not less than about 38:1, not less than about 39:1, not less than about 40:1, not less than about 41:1, not less than about 42:1, not less than about 43:1, not less than about 44:1, not less than about 45:1, not less than about 46:1, not less than about 47:1, not less than about 48:1, not less than about 49:1, or not less than about 50:1.

In another embodiment, a composition is provided that comprises a combination of at least two non-amino acid buffering agents, wherein the combination of at least two non-amino acid buffering agents comprises substantially no aluminum hydroxide-sodium bicarbonate co-precipitate. In a related embodiment, if such a composition comprises a poly[phosphoryl/sulfon]-ated carbohydrate, the weight ratio of poly[phosphoryl/sulfon]-ated carbohydrate to buffering agent is less than 1:5 (0.2), less than 1:10 (0.1) or less than 1:20 (0.05). Alternatively, the poly[phosphoryl/sulfon]-ated carbohydrate is present in the composition, if at all, in an amount less than 50 mg, less than 25 mg, less than 10 mg or less than 5 mg.

The phrase “amino acid buffering agent” as used herein includes amino acids, amino acid salts, and amino acid alkali salts including: glycine, alanine, threonine, isoleucine, valine, phenylalanine, glutamic acid, asparagininic acid, lysine, aluminum glycinate and/or lysine glutamic acid salt, glycine hydrochloride, L-alanine, DL-alanine, L-threonine, DL-threonine, L-isoleucine, L-valine, L-phenylalanine, L-glutamic acid, L-glutamic acid hydrochloride, L-glutamic acid sodium salt, L-asparaginic acid, L-asparaginic acid sodium salt, L-lysine and L-lysine-L-glutamic acid salt. The term “non-amino acid buffering agent” herein includes buffering agents as defined hereinabove but does not include amino acid buffering agents.

In another embodiment, a composition of the invention comprises at least one non-amino acid buffering agent wherein the non-amino acid buffering agent is present in the composition in a total amount greater than 800 mg. In a related embodiment, if such a composition comprises a poly[phosphoryl/sulfon]-ated carbohydrate, the weight ratio of poly[phosphoryl/sulfon]-ated carbohydrate to buffering agent is less than 1:5 (0.2), less than 1:10 (0.1) or less than 1:20 (0.05). Alternatively, the poly[phosphoryl/sulfon]-ated carbohydrate is present in the composition, if at all, in an amount less than 50 mg, less than 25 mg, less than 10 mg or less than 5 mg.

In still another embodiment, a composition is provided which comprises at least one buffering agent in a total amount of at least about 10 mEq. In a related embodiment, if an amino acid buffering agent is present in the composition, at least one of the following conditions is met: (1) the weight ratio of amino acid buffering agent:proton pump inhibitor is greater than 20:1; (2) the composition comprises at least two non-amino acid buffering agents; (3) the composition comprises at least one non-amino acid buffering agent wherein the weight ratio of the at least one non-amino acid buffering agent:proton pump inhibitor is greater than 20:1; and/or (4) the weight ratio of total buffering agent:proton pump inhibitor is greater than 40:1. In a related embodiment, the composition comprises substantially no or no amount of poly[phosphoryl/sulfon]-ated carbohydrate. In yet another related embodiment, if such a composition comprises a poly[phosphoryl/sulfon]-ated carbohydrate, the weight ratio of poly[phosphoryl/sulfon]-ated carbohydrate to buffering agent is less than 1:5 (0.2), less than 1:10 (0.1) or less than 1:20 (0.05). Alternatively, the poly[phosphoryl/sulfon]-ated carbohydrate is present in the composition in an amount less than 50 mg, less than 25 mg, less than 10 mg or less than 5 mg.

Pharmaceutical Dosage Forms

Compositions of the invention can be in the form of solid or liquid dosage forms. Non-limiting examples of suitable solid dosage forms include tablets (e.g. suspension tablets, bite suspension tablets, rapid dispersion tablets, chewable tablets, effervescent tablets, bilayer tablets, etc.), caplets, capsules (e.g. a soft or a hard gelatin capsule), powder (e.g. a packaged powder, a dispensable powder or an effervescent powder), lozenges, sachets, cachets, troches, pellets, granules, microgranules, encapsulated microgranules, powder aerosol formulations, or any other solid dosage form reasonably adapted for oral administration. In one embodiment, compositions of the invention are directed to solid final dosage forms (also referred to herein as finished dosage forms) that do not require enteric coating or further processing prior to packaging and administration to a subject. Such finished dosage forms do not include, for example, tablet or granule intermediates that are to be subsequently enteric coated or microencapsulated.

Non-limiting examples of suitable liquid dosage forms include solutions, suspension, elixirs, syrups, liquid aerosol formulations, etc. Compositions of the invention can be formulated for any method of delivery, for example oral, rectal, topical, or parenteral (e.g. subcutaneous, intramuscular, intravenous and intradermal or infusion) delivery.

In one embodiment, compositions of the invention are in the form of discrete dosage units. Such dosage units can be administered one to a small plurality (i.e. 1 to about 4) of times per day, or as many times as needed to elicit a therapeutic response. A particular dosage unit can be selected to accommodate any desired frequency of administration to achieve a specified daily dose. Typically, one dosage unit, or a small plurality (i.e. up to about 4) of dosage units, provides a sufficient amount of the PPI to result in the desired response or effect.

Individual dosage units of this embodiment typically contain about 5 mg to about 100 mg, about 7.5 mg to about 75 mg, about 10 mg to about 50 mg, about 15 mg to about 45 mg, or about 20 mg to about 40 mg of PPI. In still another embodiment, individual dosage units of the invention contain about 5 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, or about 100 mg of proton pump inhibitor.

In another embodiment, a single dosage unit of the invention comprises a therapeutically effective amount or a therapeutically and/or prophylactically effective amount of PPI. The term “therapeutically effective amount” or “therapeutically and/or prophylactically effective amount” as used herein refers to an amount of compound that is sufficient to elicit a required or desired therapeutic and/or prophylactic response, as the particular treatment context may require.

It will be understood that a therapeutically and/or prophylactically effective amount of a drug for a subject is dependent on, inter alia, the body weight, age, sex, condition and overall disease state of the subject. A “subject” herein includes a human subject of either sex and of any age, and also includes any nonhuman animal including domestic or companion animals (e.g. a cat, dog or a horse). Illustratively, where the subject is a child or a small animal (e.g., a dog) a relatively low amount of the PPI in the dose ranges provided herein will likely provide blood serum concentrations consistent with therapeutic effectiveness. Where the subject is an adult human or a large animal (e.g., a horse), achievement of such blood serum concentrations of the PPI are likely to require dose units containing a relatively greater amount of the agent.

Solid Dosage Units

In one embodiment, solid dosage units of the present invention are in the form of compressed tablets. Compressed tablets are solid dosage forms prepared by compacting a formulation containing an active ingredient and excipients selected to aid the processing and improve the properties of the product. The term “compressed tablet” generally refers to a plain, uncoated tablet for oral ingestion, prepared by a single compression or by pre-compaction tapping followed by a final compression.

Such tablets or other solid dosage forms can be prepared according to any of the many well known pharmacy techniques. In one embodiment, tablets or other solid dosage forms are prepared by processes that employ one or a combination of methods including, without limitation, (1) dry mixing, (2) direct compression, (3) milling, (4) dry or non-aqueous granulation, (5) wet granulation, or (6) fusion.

The individual steps in the wet granulation process of tablet preparation typically include milling and sieving of the ingredients, dry powder mixing, wet massing, granulation and final grinding. Dry granulation involves compressing a powder mixture into a rough tablet or “slug” on a heavy-duty rotary tablet press. The slugs are then broken up into granular particles by a grinding operation, usually by passage through an oscillation granulator. The individual steps include mixing of the powders, compressing (slugging) and grinding (slug reduction or granulation). Typically, no wet binder or moisture is involved in any of the steps.

Solid dosage forms such as tablets can also be prepared by mixing PPI with at least one pharmaceutically acceptable buffering agent as described herein above, and with one or more optional pharmaceutical excipient (in any order) to form a substantially homogeneous preformulation blend. The preformulation blend can then be subdivided and optionally further processed (e.g. compressed, encapsulated, packaged, dispersed, etc.) into any desired dosage forms.

Tablets according to the present invention may be coated or otherwise compounded to provide a dosage form affording improved handling or storage characteristics. However, any such coatings should be selected so as to not substantially delay onset of therapeutic effect of a composition of the invention upon administration to a subject (e.g. such coatings should not be effective enteric coatings).

Compositions of the invention can be prepared utilizing micronized PPI, micronized buffering agent, and/or micronized pharmaceutical excipients. Micronization is the process by which solid particles are reduced in size. Since dissolution rate is directly proportional to the surface area of a solid, and since reducing particle size of a solid increases its surface area, it is generally believed that reducing particle size of a solid will increases dissolution rate of that solid. Although micronization results in increased surface area possibly causing particle aggregation (which can negate the benefit of micronization) and is an expensive manufacturing step, it does have the significant benefit of increasing dissolution rate of relatively water insoluble drugs, such as omeprazole and other proton pump inhibitors.

In another embodiment, the present invention provides a pharmaceutical composition comprising at least one PPI and at least one buffering agent in a form convenient and stable for storage, whereby when the composition is placed into an aqueous or liquid vehicle, the composition dissolves and/or disperses yielding a solution and/or suspension suitable for oral administration to a subject. Such tablets or other solid dosage forms advantageously provide for continuous and precise dosing of a proton pump inhibitor that may be of low solubility in water and may be particularly useful for medicating children, the elderly and others who have difficulty swallowing or chewing solid dosage forms. Preferably, such dosage forms have low friability, making them easily transportable.

Suspension tablets, powder or granules are an illustrative dosage form suitable for rapid disintegration in liquid or aqueous media. The term “suspension tablet” as used herein refers to compressed tablets which rapidly disintegrate and/or disperse after being placed in water or other liquid and are thereby readily form a suspension. By providing a pharmaceutical composition including omeprazole or other proton pump inhibitor with at least one buffering agent in a solid form that can be stored and later dissolved or suspended in a prescribed amount of aqueous solution to yield the desired concentration of omeprazole and buffering agent, costs of production, shipping, and storage are greatly reduced as no liquids are shipped (reducing weight), and there is no need to refrigerate the liquid composition during transit since such final liquid composition can be prepared after shipment, for example just prior to administration to a subject. Once mixed the resultant solution can then be used to provide dosages for a single subject over a course of time, or for several subjects.

Suspension tablets can further comprise a disintegrant in addition to at least one PPI, at least one buffering agent, and optional pharmaceutical excipients. Non-limiting examples of suitable disintegrants include starches, sodium starch glycolate, clays (such as Veegum® HV), celluloses (such as purified cellulose, methylcellulose, sodium carboxymethylcellulose, and carboxymethylcellulose), alginates, pregelatinized corn starches (such as National® 1551 and National® 1550), crospovidone, and gums (such as agar, guar, locust bean, karaya, pectin, and tragacanth gums). Croscarmellose sodium, available from FMC Corporation, Philadelphia, Pa. under the trademark Ac-Di-Sol®, can be utilized in compressed tableting formulations either alone or in combination with microcrystalline cellulose to achieve rapid disintegration of the tablet.

Microcrystalline cellulose, alone or co-processed with other ingredients, is also a common additive for compressed tablets and is well known for its ability to improve compressibility of difficult to compress tablet materials. It is commercially available under the Avicel® trademark. Two different Avicel® products are utilized, Avicel® PH which is microcrystalline cellulose, and Avicel® AC-815, a co processed spray dried residue of microcrystalline cellulose and a calcium-sodium alginate complex in which the calcium to sodium ratio is in the range of about 0.40:1 to about 2.5:1. While AC-815 is comprised of 85% microcrystalline cellulose (MCC) and 15% of a calcium-sodium alginate complex, for purposes of the present invention this ratio may be varied from about 75% MCC to 25% alginate up to about 95% MCC to 5% alginate. Depending on the particular formulation and active ingredient, these two components may be present in approximately equal amounts or in unequal amounts, and either may comprise from about 1% to about 50% or about 10% to about 50% by weight of the dosage unit.

Effervescent tablets and powders are also provided by the present invention. Effervescent salts have been used to disperse medicines in water for oral administration. Effervescent salts are granules or coarse powders containing a medicinal agent in a dry mixture, usually composed of sodium bicarbonate, citric acid and tartaric acid. When the salts are added to water, the acids and the base react to liberate carbon dioxide gas, thereby causing “effervescence.”

The choice of ingredients for effervescent granules depends both upon the requirements of the manufacturing process and the necessity of making a preparation which dissolves readily in water. The two required ingredients are at least one acid and at least one base. The base releases carbon dioxide upon reaction with the acid. Examples of such acids include, but are not limited to, tartaric acid and citric acid. Preferably, the acid is a combination of both tartaric acid and citric acid. Examples of bases include, but are not limited to, sodium carbonate, potassium bicarbonate and sodium bicarbonate. Preferably, the base is sodium bicarbonate, and the effervescent combination has a pH of about 6.0 or higher.

Effervescent salts preferably include the following ingredients, which are believed to actually produce the effervescence: sodium bicarbonate, citric acid and tartaric acid. When added to water the acids and base react to liberate carbon dioxide, resulting in effervescence. It should be noted that any acid-base combination which results in the liberation of carbon dioxide could be used in place of the combination of sodium bicarbonate and citric and tartaric acids, as long as the ingredients were suitable for pharmaceutical use and result in a pH of about 6.0 or higher.

Importantly, 3 molecules of NaHCO₃ are required in order to neutralize 1 molecule of citric acid and 2 molecules of NaHCO₃ are required to neutralize 1 molecule of tartaric acid. Therefore, where an effervescent composition is desired, it is preferable that the approximate ratio of ingredients is as follows: Citric Acid:Tartaric Acid:Sodium Bicarbonate=1:2:3.44 (by weight). This ratio can be varied and continue to produce an effective release of carbon dioxide. For example, ratios of about 1:0:3 or 0:1:2 are also effective.

Effervescent granules of the present invention can be prepared according to any suitable process, for example by wet granulation, dry granulation, or fusion. The fusion method is used for the preparation of most commercial effervescent powders. Wet granulation is the oldest method of granule preparation. The individual steps in the wet granulation process of tablet preparation include milling and sieving of the ingredients; dry powder mixing; wet massing; granulation; and final grinding.

Dry granulation involves compressing a powder mixture into a rough tablet or “slug” on a heavy-duty rotary tablet press. The slugs are then broken up into granular particles by a grinding operation, usually by passage through an oscillation granulator. The individual steps include mixing of the powders; compressing (slugging); and grinding (slug reduction or granulation). No wet binder or moisture is involved in any of the steps.

The fusion method is the most preferred method for preparing the granules of the present invention. In this method, the compressing (slugging) step of the dry granulation process is eliminated. Instead, the powders are heated in an oven or other suitable source of heat.

In one embodiment, where a composition of the invention is in the form of a solid dosage unit, the weight ratio of buffering agent to PPI is greater than 20:1, for example at least about 21:1, at least about 23:1, or at least about 26:1. Illustratively, in such an embodiment, the weight ratio of buffering agent to PPI will be greater than 20:1 and less than or equal to about 150:1; greater than or equal to about 21:1 and less than or equal to about 125:1; greater than or equal to about 23:1 and less than or equal to about 100:1; or greater than 60:1 and less than or equal to about 100:1.

In another embodiment, where a composition of the invention is in the form of a solid dosage unit, the weight ratio of buffering agent to PPI is greater than 40:1, for example at least about 41:1, at least about 42:1, or at least about 43:1. Illustratively, in such an embodiment, the weight ratio of buffering agent to PPI will be greater than 40:1 and less than or equal to about 150:1; greater than or equal to about 41:1 and less than or equal to about 125:1; greater than or equal to about 42:1 and less than or equal to about 100:1; or greater than 43:1 and less than or equal to about 100:1.

Liquid Dosage Forms

In another embodiment, compositions of the present invention can be in the form of a liquid dosage form. Such compositions can be prepared in any suitable manner, for example by admixing together enteric-coated PPI granules (e.g. Prilosec® AstraZeneca) or uncoated PPI together with buffering agent, a liquid vehicle, and any other desired excipients (in any order of admixing). In one embodiment, the PPI is mixed with a pre-made solution comprising buffering agent to achieve a desired final PPI concentration. Illustratively, the concentration of PPI in the solution can range from approximately 0.2 mg/ml to about 20 mg/ml, about 0.3 mg/ml to about 15 mg/ml, or about 0.4 mg/ml to approximately 10.0 mg/ml.

Liquid dosage forms can comprise one or more optional pharmaceutical excipients including suspending agents (for example, gums, xanthans, celluloses and sugars), humectants (for example, sorbitol), solubilizers (for example, ethanol, water, PEG and propylene glycol), surfactants (for example, sodium lauryl sulfate, Spans, Tweens, and cetyl pyridine), preservatives, antioxidants (for example, parabens, vitamins E and C, and ascorbic acid), anti-caking agents, and chelating agents (for example, EDTA).

Additionally, various additives can be incorporated into such liquid dosage forms to enhance stability, sterility and/or isotonicity (e.g. sugars, sodium chloride, etc). Antimicrobial preservatives, such as ambicin, antioxidants, chelating agents, and additional buffers can be added. Various antibacterial and antifungal agents such as, for example, parabens, chlorobutanol, phenol, sorbic acid, and the like can enhance prevention of the action of microorganisms. Additionally, thickening agents such as methylcellulose can be used in order to reduce the settling of the PPI in suspension.

Liquid dosage forms may further comprise flavoring agents (e.g., chocolate, thalmantin, aspartame, peppermint, spearmint, grape, cherry, strawberry, lemon, root beer, watermelon, etc.) or other flavorings stable at pH 7 to 9, anti-foaming agents (e.g., simethicone 80 mg, Mylicon®) and parietal cell activators (discussed below).

In one embodiment, a liquid PPI composition is provided that is stable at room temperature for several weeks and that inhibits the growth of bacteria or fungi as shown in Example 10 below. In another embodiment, a liquid composition of the invention maintains greater than 90% of its PPI potency for a period of at least 12 months.

Liquid compositions of the invention can be administered to a subject via a nasogastric (ng) tube or other indwelling tubes placed in the subject's GI tract. In one embodiment, in order to avoid the critical disadvantages associated with administering large amounts of sodium bicarbonate, a composition of the invention is administered in a single dose which does not require any further administration of bicarbonate or other buffer following the administration of the composition, thereby eliminating the need for pre-or post-dose washes with additional volumes of water and sodium bicarbonate.

Pharmaceutical Excipients

Compositions of the invention can include one or more pharmaceutically acceptable excipients. Non-limiting examples of suitable excipients include suspending agents (for example, gums, xanthans, cellulosics and sugars), humectants (for example, sorbitol), solubilizers (for example, ethanol, water, PEG and propylene glycol), surfactants (for example, sodium lauryl sulfate, Spans, Tweens, and cetyl pyridine), preservatives, antioxidants (for example, parabens, and vitamins E and C), anti-caking agents, coating agents, chelating agents (for example, EDTA), stabilizers, antimicrobial agents, antifungal or antibacterial agents (for example, parabens, chlorobutanol, phenol, sorbic acid), isotonic agents (for example, sugar, sodium chloride), thickening agents (for example, methyl cellulose), flavoring agents (for example, chocolate, thalmantin, aspartame, root beer or watermelon or other flavorings stable at pH 7 to 9), anti-foaming agents (e.g., simethicone, Mylicon®), disintegrants, flow aids, lubricants, adjuvants, colorants, diluents, moistening agents, preservatives, carriers, parietal cell activators, binders (for example, hydroxypropylmethylcellulose, polyvinyl pyrilodone, other cellulosic materials and starch), diluents (for example, lactose and other sugars, starch, dicalcium phosphate and cellulosic materials), disintegrating agents (for example, starch polymers and cellulosic materials), glidants and water insoluble or water soluble lubricants or lubricating agents.

In one embodiment, compositions of the invention are in non-enteric coated form. In another embodiment, a first portion of the PPI can be enteric coated while a second portion of the PPI is non-enteric coated to provide a dual-release system. Such a composition is contemplated where both an immediate and a delayed release of the proton pump inhibiting agent into the absorption pool is desired, thereby providing an immediate and extended therapeutic effect.

In still another embodiment, the invention provides a method for preparing a pharmaceutical composition by mixing enteric coated granules of a proton pump inhibiting agent with one or more buffering agents (e.g., omeprazole 20 mg granules plus 500 mg sodium bicarbonate and 500 mg calcium carbonate) in a solid dosage form. Upon oral administration, the buffering agents elevate the gastric pH such that all or part of the enteric coating is dissolved in the gastric fluid (rather than, for example, in the higher pH environment of the duodenum), and the omeprazole is available for immediate release in the gastric fluid for absorption into the bloodstream. Many variations in this type of formulation (i.e., higher or lower amounts of inhibiting agent and/or buffering agent) may be utilized in the present invention.

Rapid Onset of Therapeutic Effect

In one embodiment, compositions of the invention are in the form of immediate release dosage units that are suitable to provide rapid onset of therapeutic effect. The term “immediate release” is intended to refer to any composition in which release of the proton pump inhibitor occurs relatively quickly after oral administration. In one immediate release embodiment, at least a therapeutically effective amount of PPI will be released from such compositions and will be available for absorption (i.e. not degraded) in the gastrointestinal tract.

In another immediate release embodiment, upon oral administration of a composition of the invention to a subject, at least a therapeutically effective amount of the active ingredient (e.g. PPI) is available for absorption in the stomach of the subject. As discussed above, commercially available PPIs require enteric coating to prevent exposure of the PPI to gastrointestinal fluids (and consequent drug degradation). Such coatings, however, by preventing release and subsequent absorption of PPI in gastrointestinal fluids, lead to delayed therapeutic onset of action. Compositions of the present embodiment, by contrast, do not require enteric coating to maintain drug stability in gastrointestinal fluids and thereby allow for rapid absorption and onset of therapeutic effect.

In one embodiment, upon oral administration of a composition of the invention to a human subject, for example a fasted adult human subject, the subject exhibits a plasma Tₘₐₓ of PPI within about 30 seconds to about 90 minutes, within about 1 minute to about 80 minutes, within about 5 minutes to about 60 minutes, within about 10 minutes to about 50 minutes, or within about 15 minutes to about 45 minutes of administration.

In another embodiment, a therapeutically-effective dose of the PPI is achieved in the blood serum of a subject at any time within about 10, about 20, about 30 or about 40 minutes from the time of administration of the composition to the subject.

In another embodiment, a therapeutically-effective dose of the PPI is achieved in the blood serum of a subject at about 20 minutes to about 12 hours, about 20 minutes to about 6 hours, about 20 minutes to about 2 hours, about 40 minutes to about 2 hours, or about 40 minutes to about 1 hour from the time of administration of the composition to the subject.

In general, a composition of the present invention is administered at a dose suitable to provide an average blood serum concentration of a proton pump inhibiting agent of at least about 1.0 μg/ml in a subject over a period of about 1 hour after administration. In one embodiment, contemplated compositions of the present invention provide a therapeutic effect as proton pump inhibiting agent medications over an interval of about 5 minutes to about 24 hours after administration, enabling once-a-day or twice-a-day administration, if desired. In another embodiment, a composition of the invention is administered at a dose suitable to provide an average blood serum concentration of a proton pump inhibiting agent of at least about 1.0 μg/ml in a subject at any time within about 10, 20, 30, or 40 minutes after administration of the composition to the subject.

In another embodiment, the present invention provides administration kits to ease mixing and administration of a composition of the invention. Illustratively, a month's supply of powder or tablets, for example, can be packaged with a separate month's supply of diluent, and a re-usable plastic dosing cup. More specifically, the package could contain thirty (30) suspension tablets containing 20 mg omeprazole each, 1 L sodium bicarbonate 8.4% solution, and a 30 ml dose cup. The user places the tablet in the empty dose cup, fills it to the 30 ml mark with the sodium bicarbonate, waits for it to dissolve (gentle stirring or agitation may be used), and then ingests the suspension. One skilled in the art will appreciate that such kits may contain many different variations of the above components. For example, if the tablets or powder are compounded to contain PPI and buffering agent, the diluent may be water, sodium bicarbonate, or other compatible diluent, and the dose cup can be larger or smaller than 30 ml in size. Also, such kits can be packaged in unit dose form, or as weekly, monthly, or yearly kits, etc.

Proton Pump Inhibitors Administered with Parietal Cell Activators

In another embodiment, the present invention provides a method for enhancing the pharmacologic activity of a proton pump inhibitor comprising co-administering with the PPI one or more parietel cell activators. The term “co-administer” and derivatives thereof means that the compound can be administered immediately before (e.g. within about 30 minutes and preferably within about 15 minutes), with, or immediately after administration of the PPI. The parietal cell activator can be formulated with or separately from the PPI.

For the purposes of this application, the term “parietal cell activator” or “activator” shall mean any compound or mixture of compounds possessing such stimulatory effect including, but not limited to, chocolate, sodium bicarbonate, calcium (e.g., calcium carbonate, calcium gluconate, calcium hydroxide, calcium acetate and calcium glycerophosphate), peppermint oil, spearmint oil, coffee, tea and colas (even if decaffeinated), caffeine, theophylline, theobromine, and amino acids (particularly aromatic amino acids such as phenylalanine and tryptophan) and combinations thereof, and the salts thereof.

Such parietal cell activators are to be administered in an amount sufficient to produce the desired stimulatory effect without causing untoward side effects to subjects. For example, chocolate, as raw cocoa, is administered in an amount of about 5 mg to 2.5 g per 20 mg dose of omeprazole (or equivalent pharmacologic dose of other proton pump inhibitor). The dose of activator administered to a mammal, particularly a human, in the context of the present invention should be sufficient to effect a therapeutic response (i.e., enhanced effect of proton pump inhibitor) over a reasonable time frame. The dose will be determined by the strength of the particular compositions employed and the condition of the person, as well as the body weight of the person to be treated. The size of the dose also will be determined by the existence, nature, and extent of any adverse side effects that might accompany the administration of a particular composition.

The approximate effective ranges for various parietal cell activators per 20 mg dose of omeprazole (or equivalent dose of other proton pump inhibitor) are:
Chocolate (raw cocoa)—5 mg to 2.5 g
Sodium bicarbonate—7 mEq to 25 mEq
Calcium carbonate—1 mg to 1.5 g
Calcium gluconate—1 mg to 1.5 g
Calcium lactate—1 mg to 1.5 g
Calcium hydroxide—1 mg to 1.5 g
Calcium acetate—0.5 mg to 1.5 g
Calcium glycerophosphate—0.5 mg to 1.5 g
Peppermint oil—(powdered form) 1 mg to 1 g
Spearmint oil—(powdered form) 1 mg to 1 g
Coffee—20 ml to 240 ml
Tea—20 ml to 240 ml
Cola—20 ml to 240 ml
Caffeine—0.5 mg to 1.5 g
Theophylline—0.5 mg to 1.5 g
Theobromine—0.5 mg to 1.5 g
Phenylalanine—0.5 mg to 1.5 g
Tryptophan—0.5 mg to 1.5 g

Pharmaceutically acceptable carriers are well-known to those who are skilled in the art. The choice of carrier will be determined, in part, both by the particular composition and by the particular method used to administer the composition. Accordingly, there is a wide variety of suitable formulations of the pharmaceutical compositions of the present invention.

Proton Pump Inhibitors Administered in Combination with other Drugs

Compositions of the invention can also be used in combination (“combination therapy”) with another pharmaceutical agent that is indicated for treating or preventing a gastrointestinal disorder, such as, for example, an anti-bacterial agent, an irritable bowel syndrome drug, a motility agent, an anti-emetic agent, an alginate, a prokinetic agent, a H₂-antagonist, or an antacid, which are commonly administered to minimize the pain and/or complications related to this disorder. Illustratively, such drugs include metoclopramide, Lotrenex®, mesalamine (5-ASA), prednisone. These drugs have certain disadvantages associated with their use. Some of these drugs are not completely effective in the treatment of the aforementioned conditions and/or produce adverse side effects, such as mental confusion, constipation, diarrhea, and thrombocytopenia. H₂-antagonists, such as ranitidine and cimetidine, are relatively costly modes of therapy, particularly in NPO patients, which frequently require the use of automated infusion pumps for continuous intravenous infusion of the drug. However, when used in combination therapy according to the present invention, many if not all of these unwanted side effects can be reduced or eliminated. The reduced side effect profile of these drugs is generally attributed to, for example, the reduced dosage necessary to achieve a therapeutic effect.

The phrase “combination therapy” embraces the administration of a composition of the present invention in conjunction with another pharmaceutical agent. In one embodiment, the agent selected for combination is indicated for treating or preventing a gastrointestinal disorder in a subject. The beneficial effect of the combination includes, but is not limited to, pharmacokinetic or pharmacodynamic co-action resulting from the combination of therapeutic agents.

Administration of therapeutic agents in combination typically is carried out over a defined time period (usually substantially simultaneously, minutes, hours, days, weeks, months or years depending upon the combination selected). “Combination therapy” generally is not intended to encompass the administration of two or more of these therapeutic agents as part of separate monotherapy regimens that incidentally and arbitrarily result in the combinations of the present invention. “Combination therapy” is intended to embrace administration of these therapeutic agents in a sequential manner, that is, where each therapeutic agent is administered at a different time, as well as administration of at least two of the therapeutic agents in a substantially simultaneous manner.

Notwithstanding the combination therapy disclosure, in one embodiment of the present invention, compositions as provided herein comprise no sucralfate, the basic aluminum hydroxide salt of sucrose octasulfate. In another embodiment, a composition of the invention is administered without co-administration of sucralfate.

Substantially simultaneous administration can be accomplished, for example, by administering to the subject a single dosage form having a fixed ratio of each therapeutic agent or in multiple, single dosage units for each of the therapeutic agents. Sequential or substantially simultaneous administration of each therapeutic agent can be effected by any appropriate route. The composition of the present invention can be administered orally or nasogastric, while the other therapeutic agent of the combination can be administered by any appropriate route for that particular agent, including, but not limited to, an oral route, a percutaneous route, an intravenous route, an intramuscular route, or by direct absorption through mucous membrane tissues.

The sequence in which the therapeutic agents are administered is not narrowly critical. “Combination therapy” also can embrace the administration of a PPI inhibitor as described herein in further combination with other biologically active agents, including, but not limited to, drugs from the following classes: abortifacients, ACE inhibitors, α- and β-adrenergic agonists, α- and β-adrenergic blockers, adrenocortical suppressants, adrenocorticotropic hormones, alcohol deterrents, aldose reductase inhibitors, aldosterone antagonists, anabolics, analgesics (including narcotic and non-narcotic analgesics), androgens, angiotensin II receptor antagonists, anorexics, antacids, anthelminthics, antiacne agents, antiallergics, antialopecia agents, antiamebics, antiandrogens, antianginal agents, antiarrhythmics, antiarteriosclerotics, antiarthritic/antirheumatic agents (including selective COX-2 inhibitors), antiasthmatics, antibacterials, antibacterial adjuncts, anticholinergics, anticoagulants, anticonvulsants, antidepressants, antidiabetics, antidiarrheal agents, antidiuretics, antidotes to poison, antidyskinetics, antieczematics, antiemetics, antiestrogens, antifibrotics, antiflatulents, antifungals, antiglaucoma agents, antigonadotropins, antigout agents, antihistaminics, antihyperactives, antihyperlipoproteinemics, antihyperphosphatemics, antihypertensives, antihyperthyroid agents, antihypotensives, antihypothyroid agents, anti-inflammatories, antimalarials, antimanics, antimethemoglobinemics, antimigraine agents, antimuscarinics, antimycobacterials, antineoplastic agents and adjuncts, antineutropenics, antiosteoporotics, antipagetics, antiparkinsonian agents, antipheochromocytoma agents, antipneumocystis agents, antiprostatic hypertrophy agents, antiprotozoals, antipruritics, antipsoriatics, antipsychotics, antipyretics, antirickettsials, antiseborrheics, antiseptics/disinfectants, antispasmodics, antisyphylitics, antithrombocythemics, antithrombotics, antitussives, antiulceratives, antiurolithics, antivenins, antiviral agents, anxiolytics, aromatase inhibitors, astringents, benzodiazepine antagonists, bone resorption inhibitors, bradycardic agents, bradykinin antagonists, bronchodilators, calcium channel blockers, calcium regulators, carbonic anhydrase inhibitors, cardiotonics, CCK antagonists, chelating agents, cholelitholytic agents, choleretics, cholinergics, cholinesterase inhibitors, cholinesterase reactivators, CNS stimulants, contraceptives, debriding agents, decongestants, depigmentors, dermatitis herpetiformis suppressants, digestive aids, diuretics, dopamine receptor agonists, dopamine receptor antagonists, ectoparasiticides, emetics, enkephalinase inhibitors, enzymes, enzyme cofactors, estrogens, expectorants, fibrinogen receptor antagonists, fluoride supplements, gastric and pancreatic secretion stimulants, gastric cytoprotectants, gastric proton pump inhibitors, gastric secretion inhibitors, gastroprokinetics, glucocorticoids, α-glucosidase inhibitors, gonad-stimulating principles, growth hormone inhibitors, growth hormone releasing factors, growth stimulants, hematinics, hematopoietics, hemolytics, hemostatics, heparin antagonists, hepatic enzyme inducers, hepatoprotectants, histamine H₂ receptor antagonists, HIV protease inhibitors, HMG CoA reductase inhibitors, immunomodulators, immunosuppressants, insulin sensitizers, ion exchange resins, keratolytics, lactation stimulating hormones, laxatives/cathartics, leukotriene antagonists, LH-RH agonists, lipotropics, 5-lipoxygenase inhibitors, lupus erythematosus suppressants, matrix metalloproteinase inhibitors, mineralocorticoids, miotics, monoamine oxidase inhibitors, mucolytics, muscle relaxants, mydriatics, narcotic antagonists, neuroprotectives, nootropics, ovarian hormones, oxytocics, pepsin inhibitors, pigmentation agents, plasma volume expanders, potassium channel activators/openers, progestogens, prolactin inhibitors, prostaglandins, protease inhibitors, radio-pharmaceuticals, 5α-reductase inhibitors, respiratory stimulants, reverse transcriptase inhibitors, sedatives/hypnotics, serenics, serotonin noradrenaline reuptake inhibitors, serotonin receptor agonists, serotonin receptor antagonists, serotonin uptake inhibitors, somatostatin analogs, thrombolytics, thromboxane A₂ receptor antagonists, thyroid hormones, thyrotropic hormones, tocolytics, topoisomerase I and II inhibitors, uricosurics, vasomodulators including vasodilators and vasoconstrictors, vasoprotectants, xanthine oxidase inhibitors, and combinations thereof.

In one embodiment, combination therapies comprise a composition useful in methods of the invention with one or more compounds described in The Merck Index, 12th Edition (1996), Therapeutic Category and Biological Activity Index, lists therein headed “Analgesic”, “Anti-inflammatory” and “Antipyretic”. Illustratively, such compounds are selected from aceclofenac, acemetacin, e-acetamidocaproic acid, acetaminophen, acetaminosalol, acetanilide, acetylsalicylic acid (aspirin), S-adenosylmethionine, alclofenac, alfentanil, allylprodine, alminoprofen, aloxiprin, alphaprodine, aluminum bis(acetylsalicylate), amfenac, aminochlorthenoxazin, 3-amino-4-hydroxybutyric acid, 2-amino-4-picoline, aminopropylon, aminopyrine, amixetrine, ammonium salicylate, ampiroxicam, antolmetin guacil, anileridine, antipyrine, antipyrine salicylate, antrafenine, apazone, bendazac, benorylate, benoxaprofen, benzpiperylon, benzydamine, benzylmorphine, bermoprofen, bezitramide, α-bisabolol, bromfenac, p-bromoacetanilide, 5-bromosalicylic acid acetate, bromosaligenin, bucetin, bucloxic acid, bucolome, bufexamac, bumadizon, buprenorphine, butacetin, butibufen, butophanol, calcium acetylsalicylate, carbamazepine, carbiphene, carprofen, carsalam, chlorobutanol, chlorthenoxazin, choline salicylate, cinchophen, cinmetacin, ciramadol, clidanac, clometacin, clonitazene, clonixin, clopirac, clove, codeine, codeine methyl bromide, codeine phosphate, codeine sulfate, cropropamide, crotethamide, desomorphine, dexoxadrol, dextromoramide, dezocine, diampromide, diclofenac sodium, difenamizole, difenpiramide, diflunisal, dihydrocodeine, dihydrocodeinone enol acetate, dihydromorphine, dihydroxyaluminum acetylsalicylate, dimenoxadol, dimepheptanol, dimethylthiambutene, dioxaphetyl butyrate, dipipanone, diprocetyl, dipyrone, ditazol, droxicam, emorfazone, enfenamic acid, epirizole, eptazocine, etersalate, ethenzamide, ethoheptazine, ethoxazene, ethylmethylthiambutene, ethylmorphine, etodolac, etofenamate, etonitazene, eugenol, felbinac, fenbufen, fenclozic acid, fendosal, fenoprofen, fentanyl, fentiazac, fepradinol, feprazone, floctafenine, flufenamic acid, flunoxaprofen, fluoresone, flupirtine, fluproquazone, flurbiprofen, fosfosal, gentisic acid, glafenine, glucametacin, glycol salicylate, guaiazulene, hydrocodone, hydromorphone, hydroxypethidine, ibufenac, ibuprofen, ibuproxam, imidazole salicylate, indomethacin, indoprofen, isofezolac, isoladol, isomethadone, isonixin, isoxepac, isoxicam, ketobemidone, ketoprofen, ketorolac, p-lactophenetide, lefetamine, levorphanol, lofentanil, lonazolac, lomoxicam, loxoprofen, lysine acetylsalicylate, magnesium acetylsalicylate, meclofenamic acid, mefenamic acid, meperidine, meptazinol, mesalamine, metazocine, methadone hydrochloride, methotrimeprazine, metiazinic acid, metofoline, metopon, mofebutazone, mofezolac, morazone, morphine, morphine hydrochloride, morphine sulfate, morpholine salicylate, myrophine, nabumetone, nalbuphine, 1-naphthyl salicylate, naproxen, narceine, nefopam, nicomorphine, nifenazone, niflumic acid, nimesulide, 5′-nitro-2′-propoxyacetanilide, norlevorphanol, normethadone, normorphine, norpipanone, olsalazine, opium, oxaceprol, oxametacine, oxaprozin, oxycodone, oxymorphone, oxyphenbutazone, papaveretum, paranyline, parsalmide, pentazocine, perisoxal, phenacetin, phenadoxone, phenazocine, phenazopyridine hydrochloride, phenocoll, phenoperidine, phenopyrazone, phenyl acetylsalicylate, phenylbutazone, phenyl salicylate, phenyramidol, piketoprofen, piminodine, pipebuzone, piperylone, piprofen, pirazolac, piritramide, piroxicam, pranoprofen, proglumetacin, proheptazine, promedol, propacetamol, propiram, propoxyphene, propyphenazone, proquazone, protizinic acid, ramifenazone, remifentanil, rimazolium metilsulfate, salacetamide, salicin, salicylamide, salicylamide o-acetic acid, salicylsulfuric acid, salsalte, salverine, simetride, sodium salicylate, sufentanil, sulfasalazine, sulindac, superoxide dismutase, suprofen, suxibuzone, talniflumate, tenidap, tenoxicam, terofenamate, tetrandrine, thiazolinobutazone, tiaprofenic acid, tiaramide, tilidine, tinoridine, tolfenamic acid, tolmetin, tramadol, tropesin, viminol, xenbucin, ximoprofen, zaltoprofen and zomepirac.

Where an antacid is desired as part of a combination therapy, the antacid can include, but is not limited to, alexitol sodium, almagate, aluminum hydroxide, aluminum magnesium silicate, aluminum phosphate, azulene, basic aluminum carbonate gel, bismuth aluminate, bismuth phosphate, bismuth subgallate, bismuth subnitrate, dihydroxyaluminum aminoacetate, dihydroxyaluminum sodium carbonate, ebimar, magaldrate, magnesium carbonate hydroxide, magnesium oxide, magnesium peroxide, magnesium phosphate tribasic, magnesium silicate, potassium citrate, and combinations thereof. (Based in part upon the list provided in The Merck Index, Merck & Co. Rahway, N.J. (2001)).

The therapeutic compounds which make up the combination therapy may be a combined dosage form or in separate dosage forms intended for substantially simultaneous administration. The therapeutic compounds that make up the combination therapy may also be administered sequentially, with either therapeutic compound being administered by a regimen calling for two step administration. Thus, a regimen may call for sequential administration of the therapeutic compounds with spaced-apart administration of the separate, active agents. The time period between the multiple administration steps may range from, for example, a few minutes to several hours to days, depending upon the properties of each therapeutic compound such as potency, solubility, bioavailability, plasma half-life and kinetic profile of the therapeutic compound, as well as depending upon the effect of food ingestion and the age and condition of the subject. Circadian variation of the target molecule concentration may also determine the optimal dose interval. The therapeutic compounds of the combined therapy whether administered simultaneously, substantially simultaneously, or sequentially, may involve a regimen calling for administration of one therapeutic compound by oral route and another therapeutic compound by an oral route, a percutaneous route, an intravenous route, an intramuscular route, or by direct absorption through mucous membrane tissues, for example. Whether the therapeutic compounds of the combined therapy are administered orally, by inhalation spray, rectally, topically, buccally (for example, sublingual), or parenterally (for example, subcutaneous, intramuscular, intravenous and intradermal injections, or infusion techniques), separately or together, each such therapeutic compound will be contained in a suitable pharmaceutical formulation of pharmaceutically-acceptable excipients, diluents or other formulations components.

Compositions of the present invention are suitable for, inter alia, treating a gastrointestinal disorder in a subject in need thereof, illustratively by orally administering such a composition to subject in need thereof. Compositions of the invention are suitable for treating an acid related gastrointestinal disorder in a subject in need thereof, for example by orally administering to the subject a pharmaceutical composition of the invention.

### EXAMPLES

The present invention is further illustrated by the following examples, which should not be construed as limiting in any way.

### Example I

A. Fast Disintegrating Suspension Tablets of Omeprazole.

A fast disintegrating tablet is compounded as follows: Croscarmellose sodium 300 g is added to the vortex of a rapidly stirred beaker containing 3.0 kg of deionized water. This slurry is mixed for 10 minutes. Omeprazole 90 g (powdered) is placed in the bowl of a Hobart mixer. After mixing, the slurry of croscarmellose sodium is added slowly to the omeprazole in the mixer bowl, forming a granulation, which is then placed in trays and dried at 70° C. for three hours. The dry granulation is then placed in a blender, and to it is added 1,500 g of Avicel® AC-815 (85% microcrystalline cellulose coprocessed with 15% of a calcium, sodium alginate complex) and 1,500 g of Avicel® PH-302 (microcrystalline cellulose). After this mixture is thoroughly blended, 35 g of magnesium stearate is added and mixed for 5 minutes. The resulting mixture is compressed into tablets on a standard tablet press (Hata HS). These tablets have an average weight of about 0.75 g, and contain about 20 mg omeprazole. These tablets have low friability and rapid disintegration time. This formulation may be dissolved in an aqueous solution containing a buffering agent for immediate oral administration.

Alternatively, the suspension tablet may be swallowed whole with a solution of buffering agent. In both cases, the preferred solution is sodium bicarbonate 8.4%. As a further alternative, sodium bicarbonate powder (about 975 mg per 20 mg dose of omeprazole (or an equipotent amount of other proton pump inhibitor) is compounded directly into the tablet. Such tablets are then dissolved in water or sodium bicarbonate 8.4%, or swallowed whole with an aqueous diluent.

| B1. 10 mg Tablet Formula. | | |
|---|---|---|
| Omeprazole | 10 | mg (or lansoprazole or |
| pantoprazole | | or other proton pump |
| in an | | inhibitor equipotent |
| | | amount) |
| Calcium lactate | 175 | mg |
| Calcium glycerophosphate | 175 | mg |
| Sodium bicarbonate | 250 | mg |
| Aspartame calcium | 0.5 | mg |
| (phenylalanine) | | |
| Colloidal silicon dioxide | 12 | mg |
| Corn starch | 15 | mg |
| Croscarmellose sodium | 12 | mg |
| Dextrose | 10 | mg |
| Peppermint | 3 | mg |
| Maltodextrin | 3 | mg |
| Mannitol | 3 | mg |
| Pregelatinized starch | 3 | mg |
| B2. 10 mg Tablet Formula. | | |
| Proton pump inhibitor: one of the following: | | |
| Omeprazole | 10 | mg |
| Lansoprazole | 15 | mg |
| Pantoprazole sodium | 20 | mg |
| Rabeprazole sodium | 10 | mg |
| Other proton pump inhibitor in an equipotent amount | | |
| Calcium lactate | 375 | mg |
| Calcium glycerophosphate | 375 | mg |
| Aspartame calcium | 0.5 | mg |
| (phenylalanine) | | |
| Colloidal silicon dioxide | 12 | mg |
| Corn starch | 15 | mg |
| Croscarmellose sodium | 12 | mg |
| Dextrose | 10 | mg |
| Peppermint | 3 | mg |
| Maltodextrin | 20 | mg |
| Mannitol | 30 | mg |
| Pregelatinized starch | 30 | mg |
| B3. 10 mg Tablet Formula. | | |
| Proton pump inhibitor: one of the following: | | |
| Omeprazole | 10 | mg |
| Lansoprazole | 15 | mg |
| Pantoprazole sodium | 20 | mg |
| Rabeprazole sodium | 10 | mg |
| Other proton pump inhibitor in an equipotent amount | | |
| Sodium bicarbonate | 750 | mg |
| Aspartame sodium | 0.5 | mg |
| (phenylalanine) | | |
| Colloidal silicon | 12 | mg |
| dioxide | | |
| Corn starch | 15 | mg |
| Croscarmellose sodium | 12 | mg |
| Dextrose | 10 | mg |
| Peppermint | 3 | mg |
| Maltodextrin | 20 | mg |
| Mannitol | 30 | mg |
| Pregelatinized starch | 30 | mg |
| C1. 20 mg Tablet Formula. | | |
| Omeprazole | 20 | mg (or lansoprazole or |
| or | | pantoprazole other proton |
| | | pump inhibitor in an |
| | | equipotent amount) |
| Calcium lactate | 175 | mg |
| Calcium glycerophosphate | 175 | mg |
| Sodium bicarbonate | 250 | mg |
| Aspartame calcium | 0.5 | mg |
| (phenylalanine) | | |
| Colloidal silicon | 12 | mg |
| dioxide | | |
| Corn starch | 15 | mg |
| Croscarmellose sodium | 12 | mg |
| Dextrose | 10 | mg |
| Calcium hydroxide | 10 | mg |
| Peppermint | 3 | mg |
| Maltodextrin | 3 | mg |
| Mannitol | 3 | mg |
| Pregelatinized starch | 3 | mg |
| C2. 20 mg Tablet Formula. | | |
| Proton pump inhibitor: One of the following: | | |
| Omeprazole | 20 | mg |
| Lansoprazole | 30 | mg |
| Pantoprazole | 40 | mg |
| Other proton pump inhibitor in an equipotent amount | | |
| Calcium lactate | 375 | mg |
| Calcium glycerophosphate | 375 | mg |
| Aspartame calcium | 0.5 | mg |
| (phenylalanine) | | |
| Colloidal silicon dioxide | 12 | mg |
| Corn starch | 15 | mg |
| Croscarmellose sodium | 12 | mg |
| Dextrose | 10 | mg |
| Peppermint | 3 | mg |
| Maltodextrin | 20 | mg |
| Mannitol | 30 | mg |
| Pregelatinized starch | 30 | mg |
| C3. 20 mg Tablet Formula. | | |
| Proton pump inhibitor: One of the following: | | |
| Omeprazole | 20 | mg |
| Lansoprazole | 30 | mg |
| Pantoprazole | 40 | mg |
| Other proton pump inhibitor in an equipotent amount | | |
| Sodium bicarbonate | 750 | mg |
| Aspartame sodium | 0.5 | mg |
| (phenylalanine) | | |
| Colloidal silicon | 12 | mg |
| dioxide | | |
| Corn starch | 15 | mg |
| Croscarmellose sodium | 12 | mg |
| Dextrose | 10 | mg |
| Peppermint | 3 | mg |
| Maltodextrin | 20 | mg |
| Mannitol | 30 | mg |
| Pregelatinized starch | 30 | mg |
| D1. Tablet for Rapid Dissolution. | | |
| Omeprazole | 20 | mg (or lansoprazole or |
| or | | pantoprazole other proton |
| | | pump inhibitor in an |
| | | equipotent amount) |
| Calcium lactate | 175 | mg |
| Calcium glycerophosphate | 175 | mg |
| Sodium bicarbonate | 500 | mg |
| Calcium hydroxide | 50 | mg |
| Croscarmellose sodium | 12 | mg |
| D2. Tablet for Rapid Dissolution. | | |
| Proton pump inhibitor: One of the following: | | |
| Omeprazole | 20 | mg |
| Lansoprazole | 30 | mg |
| Pantoprazole | 40 | mg |
| Rabeprazole sodium | 20 | mg |
| Esomeprazole magnesium | 20 | mg |
| Other proton pump inhibitor in an equipotent amount | | |
| Calcium lactate | 300 | mg |
| Calcium glycerophosphate | 300 | mg |
| Calcium hydroxide | 50 | mg |
| Croscarmellose sodium | 12 | mg |
| D3. Tablet for Rapid Dissolution. | | |
| Proton pump inhibitor: One of the following: | | |
| Omeprazole | 20 | mg |
| Lansoprazole | 30 | mg |
| Pantoprazole | 40 | mg |
| Rabeprazole sodium | 20 | mg |
| Esomeprazole magnesium | 20 | mg |
| Other proton pump inhibitor in an equipotent amount | | |
| Sodium bicarbonate | 700 | mg |
| Trisodium phosphate | 100 | mg |
| dodecahydrate | | |
| Croscarmellose sodium | 12 | mg |
| E1. Powder for Reconstitution for Oral Use (or per ng tube). | | |
| Omeprazole | 20 | mg (or lansoprazole or |
| or | | pantoprazole other proton |
| | | pump inhibitor in an |
| | | equipotent amount) |
| Calcium lactate | 175 | mg |
| Calcium glycerophosphate | 175 | mg |
| Sodium bicarbonate | 500 | mg |
| Calcium hydroxide | 50 | mg |
| Glycerine | 200 | mg |
| E2. Powder for Reconstitution for Oral Use (or per ng tube). | | |
| Proton pump inhibitor: One of the following: | | |
| Omeprazole | 20 | mg |
| Lansoprazole | 30 | mg |
| Pantoprazole | 40 | mg |
| Rabeprazole sodium | 20 | mg |
| Esomeprazole magnesium | 20 | mg |
| Other proton pump inhibitor in an equipotent amount | | |
| Calcium lactate | 300 | mg |
| Calcium glycerophosphate | 300 | mg |
| Calcium hydroxide | 50 | mg |
| Glycerine | 200 | mg |
| E3. Powder for Reconstitution for Oral Use (or per ng tube). | | |
| Proton pump inhibitor: One of the following: | | |
| Omeprazole | 20 | mg |
| Lansoprazole | 30 | mg |
| Pantoprazole | 40 | mg |
| Rabeprazole sodium | 20 | mg |
| Esomeprazole magnesium | 20 | mg |
| Other proton pump inhibitor in an equipotent amount | | |
| Sodium bicarbonate | 850 | mg |
| Trisodium phosphate | 50 | mg |
| F1. 10 mg Tablet Formula. | | |
| Omeprazole | 10 | mg (or lansoprazole or |
| | | pantoprazole or other |
| | | proton pump inhibitor |
| | | in an equipotent at mount) |
| Calcium lactate | 175 | mg |
| Calcium glycerophosphate | 175 | mg |
| Sodium bicarbonate | 250 | mg |
| Polyethylene glycol | 20 | mg |
| Croscarmellose sodium | 12 | mg |
| Peppermint | 3 | mg |
| Magnesium silicate | 1 | mg |
| Magnesium stearate | 1 | mg |
| F2. 10 mg Tablet Formula. | | |
| Proton pump inhibitor: One of the following: | | |
| Omeprazole | 10 | mg |
| Lansoprazole | 15 | mg |
| Pantoprazole sodium | 20 | mg |
| Rabeprazole sodium | 10 | mg |
| Esomeprazole magnesium | 10 | mg |
| Other proton pump inhibitor in an equipotent amount | | |
| Calcium lactate | 475 | mg |
| Calcium glycerophosphate | 250 | mg |
| Polyethylene glycol | 20 | mg |
| Croscarmellose sodium | 12 | mg |
| Peppermint | 3 | mg |
| Magnesium silicate | 10 | mg |
| Magnesium stearate | 10 | mg |
| F3. 10 mg Tablet Formula. | | |
| Proton pump inhibitor: One of the following: | | |
| Omeprazole | 10 | mg |
| Lansoprazole | 15 | mg |
| Pantoprazole sodium | 20 | mg |
| Rabeprazole sodium | 10 | mg |
| Esomeprazole magnesium | 10 | mg |
| Other proton pump inhibitor in an equipotent amount | | |
| Sodium bicarbonate | 700 | mg |
| Polyethylene glycol | 20 | mg |
| Croscarmellose sodium | 12 | mg |
| Peppermint | 3 | mg |
| Magnesium silicate | 10 | mg |
| Magnesium stearate | 10 | mg |
| G1. 10 mg Tablet Formula. | | |
| Omeprazole | 10 | mg (or lansoprazole or |
| | | pantoprazole or other |
| | | proton pump inhibitor |
| | | in an equipotent amount) |
| Calcium lactate | 200 | mg |
| Calcium glycerophosphate | 200 | mg |
| Sodium bicarbonate | 400 | mg |
| Croscarmellose sodium | 12 | mg |
| Pregelatinized starch | 3 | mg |
| G2. 10 mg Tablet Formula. | | |
| Proton pump inhibitor: One of the following: | | |
| Omeprazole | 10 | mg |
| Lansoprazole | 15 | mg |
| Pantoprazole sodium | 20 | mg |
| Rabeprazole sodium | 10 | mg |
| Esomeprazole magnesium | 10 | mg |
| Other proton pump inhibitor in an equipotent amount | | |
| Calcium lactate | 400 | mg |
| Calcium glycerophosphate | 400 | mg |
| Croscarmellose sodium | 12 | mg |
| Pregelatinized starch | 3 | mg |
| G3. 10 mg Tablet Formula. | | |
| Proton pump inhibitor: One of the following: | | |
| Omeprazole | 10 | mg |
| Lansoprazole | 15 | mg |
| Pantoprazole sodium | 20 | mg |
| Rabeprazole sodium | 10 | mg |
| Esomeprazole magnesium | 10 | mg |
| Other proton pump inhibitor in an equipotent amount | | |
| Sodium bicarboante | 750 | mg |
| Croscarmellose sodium | 12 | mg |
| Pregelatinized starch | 3 | mg |

All of the tablets and powders of this Example may be swallowed whole, chewed or mixed with an aqueous medium prior to administration.

### Example II

Standard Tablet of Proton Pump Inhibitor and Buffering Agent.

Ten (10) tablets were prepared using a standard tablet press, each tablet comprising about 20 mg omeprazole and about 975 mg sodium bicarbonate uniformly dispersed throughout the tablet. To test the disintegration rate of the tablets, each was added to 60 ml of water. Using previously prepared liquid omeprazole/sodium bicarbonate solution as a visual comparator, it was observed that each tablet was completely dispersed in under three (3) minutes.

Another study using the tablets compounded according to this Example evaluated the bioactivity of the tablets in five (5) adult critical care subjects. Each subject was administered one tablet via ng with a small amount of water, and the pH of ng aspirate was monitored using paper measure. The pH for each subject was evaluated for 6 hours and remained above 4, thus demonstrating the therapeutic benefit of the tablets in these patients.

Tablets were also prepared by boring out the center of sodium bicarbonate USP 975 mg tablets with a knife. Most of the removed sodium bicarbonate powder was then triturated with the contents of a 20 mg Prilosec® capsule and the resulting mixture was then packed into the hole in the tablet and sealed with glycerin.

### Example III

Proton Pump Inhibitor Central Core Tablet.

Tablets are prepared in a two-step process. First, about 20 mg of omeprazole is formed into a tablet as is known in the art to be used as a central core. Second, about 975 mg sodium bicarbonate USP is used to uniformly surround the central core to form an outer protective cover of sodium bicarbonate. The central core and outer cover are both prepared using standard binders and other excipients to create a finished, pharmaceutically acceptable tablet. The tablets may be swallowed whole with a glass of water.

### Example IV

Effervescent Tablets and Granules.

The granules of one 20 mg Prilosec® capsule were emptied into a mortar and triturated with a pestle to a fine powder. The omeprazole powder was then geometrically diluted with about 958 mg sodium bicarbonate USP, about 832 mg citric acid USP and about 312 mg potassium carbonate USP to form a homogeneous mixture of effervescent omeprazole powder. This powder was then added to about 60 ml of water whereupon the powder reacted with the water to create effervescence. A bubbling solution resulted of omeprazole and principally the antacids sodium citrate and potassium citrate. The solution was then administered orally to one adult male subject and gastric pH was measured using pHydrion paper. The results were as follows:

| Time Interval | pH Measured |
|---|---|
| Immediately prior to dose | 2 |
| 1 hour post dose | 7 |
| 2 hours post dose | 6 |
| 4 hours post dose | 6 |
| 6 hours post dose | 5 |
| 8 hours post dose | 4 |

One skilled in the art of pharmaceutical compounding will appreciate that bulk powders can be manufactured using the above ratios of ingredients, and that the powder can be pressed into tablets using standard binders and excipients. Such tablets are then mixed with water to activate the effervescent agents and create the desired solution. In addition, lansoprazole 30 mg (or an equipotent dose of other proton pump inhibitor) can be substituted for omeprazole.

The effervescent powder and tablets can alternatively be formulated by employing the above mixture but adding an additional 200 mg of sodium bicarbonate USP to create a resulting solution with a higher pH. Further, instead of the excess 200 mg of sodium bicarbonate, 100 mg of calcium glycerophosphate or 100 mg of calcium lactate can be employed. Combinations of the same can also added.

### Example V

Parietal Cell Activator “Choco-Base™™” Formulations and Efficacy.

Children are affected by gastro esophageal reflux disease (GERD) with atypical manifestations. Many of these atypical symptoms are difficult to control with traditional drugs such as H₂-antagonists, cisapride, or sucralfate. Proton pump inhibiting agents are more effective in controlling gastric pH and the symptoms of gastroesophageal reflux disease than other agents. However, proton pump inhibiting agents are not available in dosage forms that are easy to administer to young children. To address this problem, applicant employed omeprazole or lansoprazole in a buffered chocolate suspension (Choco-Base™), in children with manifestations of gastroesophageal reflux disease.

Applicant performed a retrospective evaluation of children with gastroesophageal reflux disease referred to the University of Missouri-Columbia from 1995 to 1998 who received treatment with the experimental omeprazole or lansoprazole Choco-Base™ suspension formulated in accordance with Formulation 1 stated below. Data were included on all patients with follow up information sufficient to draw conclusions about pre/post treatment (usually >6 months). There were 25 patients who met the criteria for this evaluation. Age range was several weeks to greater than 5 years. Most patients had a history of numerous unsuccessful attempts at ameliorating the effects of gastroesophageal reflux disease. Medication histories indicated many trials of various drugs.

The primary investigator reviewed all charts for uniformity of data collection. When insufficient data was available in the University charts, attempts were made to review charts in the local primary care physicians offices for follow-up data. If information was still unavailable to review, attempts were made to contact family for follow-up. If data were still unavailable the patients were considered inevaluable.

Patient charts were reviewed in detail. Data noted were date of commencement of therapy, date of termination of therapy and any reason for termination other than response to treatment. Patient demographics were also recorded, as were any other medical illnesses. Medical illnesses were divided grossly into those that are associated with or exacerbate gastroesophageal reflux disease and those that do not.

Patient charts were examined for evidence of response to therapy. As this was largely a referral population, and a retrospective review, quantification of symptomatology based on scores, office visits and ED visits was difficult. Therefore, applicant examined charts for evidence of an overall change in patient symptoms. Any data to point towards improvement, decline or lack of change were examined and recorded.

Results.

A total of 33 pediatric patients to date have been treated with the above-described suspension at the University of Missouri-Columbia. Of the 33 patients, 9 were excluded from the study, all based upon insufficient data about commencement, duration or outcome in treatment with proton pump inhibitor therapy. This left 24 patients with enough data to draw conclusions.

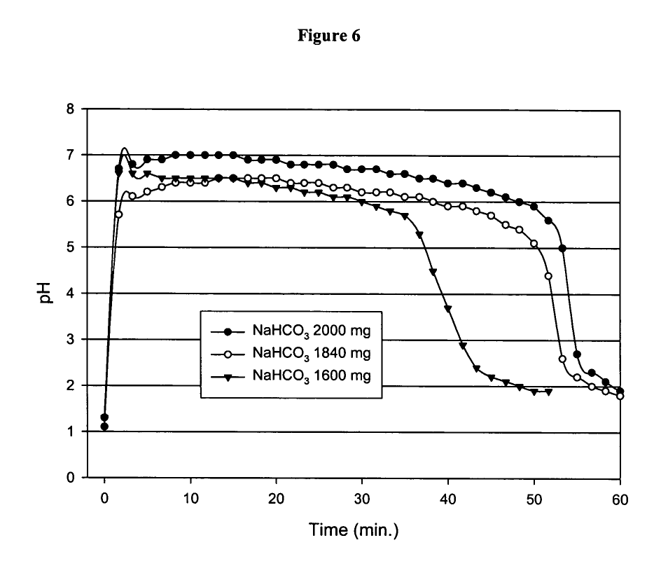

Of the 24 remaining patients, 18 were males and 6 females. Ages at implementation of proton pump inhibitor therapy ranged from 2 weeks of age to 9 years old. Median age at start of therapy was 26.5 months [mean of 37 mo.]. Early on, reflux was usually documented by endoscopy and confirmed by pH probe. Eventually, pH probe was dropped and endoscopy was the sole method for documenting reflux, usually at the time of another surgery (most often T-tubes or adenoidectomy). Seven patients had pH probe confirmation of gastroesophageal reflux disease, whereas 18 had endoscopic confirmation of reflux including all eight who had pH probing done (See FIG. 5 and 6). Reflux was diagnosed on endoscopy most commonly by cobblestoning of the tracheal wall, with laryngeal and pharyngeal cobblestoning as findings in a few patients. Six patients had neither pH nor endoscopic documentation of gastroesophageal reflux disease, but were tried on proton pump inhibitor therapy based on symptomatology alone.

Past medical history was identified in each chart. Ten patients had reflux-associated diagnoses. These were most commonly cerebral palsy, prematurity and Pierre Robin sequence. Other diagnoses were Charcot-Marie-Tooth disease, Velocardiofacial syndrome, Down syndrome and De George's syndrome. Non-reflux medical history was also identified and recorded separately (See Table 2 below).

Patients were, in general, referral patients from local family practice clinics, pediatricians, or other pediatric health care professionals. Most patients were referred to ENT for upper airway problems, sinusitis, or recurrent/chronic otitis media that had been refractory to medical therapy as reported by the primary care physician. Symptoms and signs most commonly found in these patients were recorded and tallied. All signs and symptoms were broken down into six major categories: (1) nasal; (2) otologic; (3) respiratory; (4) gastrointestinal; (5) sleep-related; and (6) other. The most common problems fell into one or all of the first 3 categories (See Table 1 below).

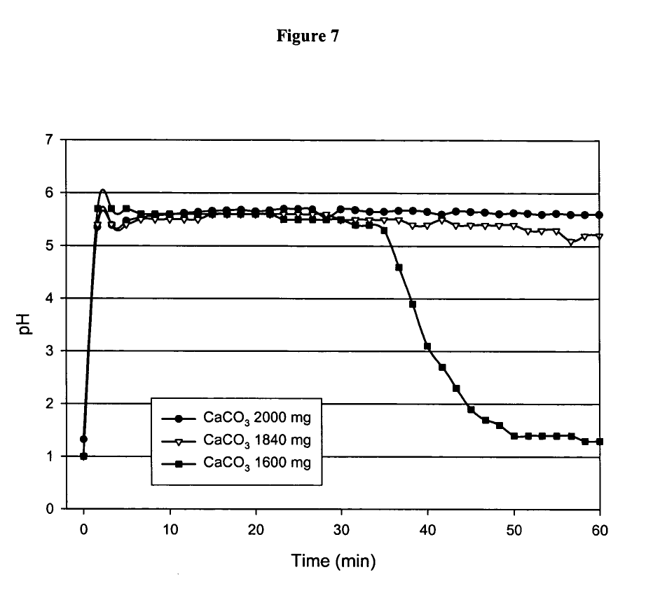

Most patients had been treated in the past with medical therapy in the form of antibiotics, steroids, asthma medications and other diagnosis-appropriate therapies. In addition, nine of the patients had been on reflux therapy in the past, most commonly in the form of conservative therapy such as head of bed elevation 30°, avoidance of evening snacks, avoidance of caffeinated beverages as well as cisapride and ranitidine (See FIG. 7).

The proton pump inhibitor suspension used in this group of patients was Choco-Base™ suspension of either lansoprazole or omeprazole. The dosing was very uniform, with patients receiving doses of either 10 or 20 mg of omeprazole and 23 mg of lansoprazole. Initially, in April of 1996 when therapy was first instituted 10 mg of omeprazole was used. There were 3 patients in this early phase who were treated initially with 10 mg po qd of omeprazole. All three subsequently were increased to either 20 mg po qd of omeprazole or 23 mg po qd of lansoprazole. All remaining patients were given either the 20 mg omeprazole or the 23 mg lansoprazole treatment qd, except in one case, where 30 mg of lansoprazole was used. Patients were instructed to take their doses once per day, preferably at night in most cases. Suspensions were all filled through the University of Missouri Pharmacy at Green Meadows. This allowed for tracking of usage through refill data.

Most patients responded favorably to and tolerated the once daily dosing of Choco-Base™ proton pump inhibitor suspension. Two patients had documented adverse effects associated with the use of the proton pump inhibitor suspension. In one patient, the mother reported increased burping up and dyspepsia, which was thought to be related to treatment failure. The other patient had small amounts of bloody stools per mother. This patient never had his stool tested, as his bloody stool promptly resolved upon cessation of therapy, with no further sequellae. The other 23 patients had no documented adverse effects.

Patients were categorized based on review of clinic notes and chart review into general categories: (1) improved; (2) unchanged; (3) failed; and (4) inconclusive. Of 24 patients with sufficient data for follow up, 18 showed improvement in symptomatology upon commencement of proton pump inhibitor therapy [72%]. The seven who did not respond were analyzed and grouped. Three showed no change in symptomatology and clinical findings while on therapy, one complained of worsening symptoms while on therapy, one patient had therapy as prophylaxis for surgery, and two stopped therapy just after its commencement (see FIG. 8). Setting aside the cases in which therapy was stopped before conclusions could be drawn and the case in which proton pump inhibitor therapy was for purely prophylactic reasons, leaves (17/21) 81 % of patients that responded to Choco-Base™ suspension. This means that 19% (4/21) of patients received no apparent benefit from proton pump inhibitor therapy. Of all these patients, only 4% complained of worsening symptoms and the side effects were 4% (1/21) and were mild bloody stool that completely resolved upon cessation of therapy.

Discussion.

Gastroesophageal reflux disease in the pediatric population is relatively common, affecting almost 50% of newborns. Even though most infants outgrow physiologic reflux, pathologic reflux still affects approximately 5% of all children throughout childhood. Recently considerable data has pointed to reflux as an etiologic factor in extra-esophageal areas, gastroesophageal reflux disease has been attributed to sinusitis, dental caries, otitis media, asthma, apnea, arousal, pneumonia, bronchitis, and cough, among others. Despite the common nature of reflux, there seems to have been little improvement in therapy for reflux, especially in the non-surgical arena.

The standard of therapy for the treatment of gastroesophageal reflux disease in the pediatric population has become a progression from conservative therapy to a combination of a pro-kinetic agent and H-2 blocker therapy. Nonetheless, many patients fail this treatment protocol and become surgical candidates. In adults, proton pump inhibitor therapy is effective in 90% of those treated for gastroesophageal reflux disease. As a medical alternative to the H-2 blockers, the proton pump inhibiting agents have not been studied extensively in the pediatric population. Part of the reason for this lack of data may be related to the absence of a suitable dosage formulation for this very young population, primarily under 2 years of age, that does not swallow capsules or tablets. It would be desirable to have a true liquid formulation (solution or suspension) with good palatability such as is used for oral antibiotics, decongestants, antihistamines, H-2 blockers, cisapride, metoclopramide, etc. The use of lansoprazole granules (removed from the gelatin capule) and sprinkled on applesauce has been approved by the Food and Drug Administration as an alternative method of drug administration in adults but not in children. Published data are lacking on the efficacy of the lansoprazole sprinkle method in children. Omeprazole has been studied for bioequivalence as a sprinkle in adults and appears to produce comparable serum concentrations when compared to the standard capsule. Again no data are available on the omeprazole sprinkle in children. An additional disadvantage of omeprazole is its taste which is quinine-like. Even when suspended in juice, applesauce or the like, the bitter nature of the medicine is easily tasted even if one granule is chewed. For this reason applicant eventually progressed to use lansoprazole in Choco-Base™. Pantoprazole and rabeprazole are available as enteric-coated tablets only. Currently, none of the proton pump inhibiting agents available in the United States are approved for pediatric use. There is some controversy as to what the appropriate dosage should be in this group of patients. A recent review by Israel D., et al. suggests that effective proton pump inhibitor dosages should be higher than that originally reported, i.e., from 0.7 mg/kg to 2 or 3 mg/kg omeprazole. Since toxicity with the proton pump inhibiting agents is not seen even at >50 mg/kg, there appears little risk associated with the higher dosages. Based on observations at the University of Missouri consistent with the findings of this review, applicant established a simple fixed dosage regimen of 10 ml Choco-Base™ suspension daily. This 10 ml dose provided 20 mg omeprazole or 23 mg lansoprazole.

In the ICU setting, the University of Missouri-Columbia has been using an unflavored proton pump inhibitor suspension given once daily per various tubes (nasogastric, g-tube, jejunal feeding tube, duo tube, etc.) for stress ulcer prophylaxis. It seemed only logical that if this therapy could be made into a palatable form, it would have many ideal drug characteristics for the pediatric population. First, it would be liquid, and therefore could be administered at earlier ages. Second, if made flavorful it could help to reduce noncompliance. Third, it could afford once daily dosing, also helping in reducing noncompliance. In the process, applicant discovered that the dosing could be standardized, which nearly eliminated dosing complexity.

Choco-Base™ is a product which protects drugs which are acid labile, such as proton pump inhibiting agents, from acid degradation. The first few pediatric patients with reflux prescribed Choco-Base™ were sicker patients. They had been on prior therapy and had been diagnosed both by pH probe and endoscopy. In the first few months, applicant treated patients with 10 mg of omeprazole qd (1 mg/kg) and found this to be somewhat ineffective, and quickly increased the dosing to 20 mg (2 mg/kg) of omeprazole. About halfway through the study, applicant began using lansoprazole 23 mg po qd. Applicant's standard therapy was then either 20 mg of omeprazole or 23 mg of lansoprazole once daily. The extra 3 mg of lansoprazole is related only to the fact that the final concentration was 2.25 mg/ml, and applicant desired to keep dosing simple, so he used a 10 ml suspension.

The patients that were treated represented a tertiary care center population, and they were inherently sicker and refractory to medical therapy in the past. The overall 72% success rate is slightly lower than the 90% success rates of proton pump inhibiting agents in the adult population, but this can be attributed to the refractory nature of their illness, most having failed prior non- proton pump inhibitor treatment. The population in this study is not indicative of general practice populations.

Conclusion.

Proton pump inhibitor therapy is a beneficial therapeutic option in the treatment of reflux related symptoms in the pediatric population. Its once daily dosing and standard dosing scheme combined with a palatable formulation makes it an ideal pharmacologic agent.

TABLE 1

| Symptoms | Patient Numbers |
|---|---|
| Nasal: | 35 |
| Sinusitis | 7 |
| Congestion | 8 |
| Nasal discharge | 16 |
| Other | 4 |
| Otologic: | 26 |
| Otitis Media | 17 |
| Otorrhea | 9 |
| Respiratory: | 34 |
| Cough | 10 |
| Wheeze | 11 |
| Respiratory Distress: | 5 |
| Pneumonia | 2 |
| Other | 6 |
| Gastrointestinal: | 10 |
| Abdominal Pain | 1 |
| Reflux/Vomiting | 4 |
| Other | 4 |
| Sleep Disturbances: | 11 |
| Other | 2 |

TABLE 2

| Past Medical History | Number of Patients |
|---|---|
| Reflux Associated: | 12 |
| Premature | 5 |
| Pierre-Robin | 2 |
| Cerebral Palsy | 2 |
| Down Syndrome | 1 |
| Charcot-Marie-Tooth | 1 |
| Velocardiofacial Syndrome | 1 |
| Other Medical History | 12 |
| Cleft Palate | 3 |
| Asthma | 3 |
| Autism | 2 |
| Seizure Disorder | 1 |
| Diabetes Mellitus | 1 |
| Subglottic Stenosis | 1 |
| Tracheostomy Dependent | 1 |

The Choco-Base™ product is formulated as follows:

FORMULATION 1

| PART A INGREDIENTS | AMOUNT (mg) |
|---|---|
| Omeprazole | 200 |
| Sucrose | 26000 |
| Sodium Bicarbonate | 9400 |
| Cocoa | 1800 |
| Corn Syrup Solids | 6000 |
| Sodium Caseinate | 1000 |
| Soy Lecithin | 150 |
| Sodium Chloride | 35 |
| Tricalcium Phosphate | 20 |
| Dipotassium Phosphate | 12 |
| Silicon Dioxide | 5 |
| Sodium Stearoyl Lactylate | 5 |

| PART B INGREDIENTS | AMOUNT (ml) |
|---|---|
| Distilled Water | 100 |
| COMPOUNDING INSTRUCTIONS | |
| Add Part B to Part A to create a total | |
| volume of approximately 130 ml with an | |
| omeprazole concentration of about 1.5 mg/ml. | |

FORMULATION 2

| PART A INGREDIENTS (mg) | AMOUNT (mg) |
|---|---|
| Sucrose | 26000 |
| Cocoa | 1800 |
| Corn Syrup Solids | 6000 |
| Sodium Caseinate | 1000 |
| Soy Lecithin | 150 |
| Sodium Chloride | 35 |
| Tricalcium Phosphate | 20 |
| Dipotassium Phosphate | 12 |
| Silicon Dioxide | 5 |
| Sodium Stearoyl Lactylate | 5 |

| PART B INGREDIENTS | AMOUNT | |
|---|---|---|
| Distilled Water | 100 | ml |
| Sodium Bicarbonate | 8400 | mg |
| Omeprazole | 200 | mg |
| COMPOUNDING INSTRUCTIONS | | |
| Mix the constituents of Part B together | | |
| thoroughly and then add to Part A. | | |
| This results in a total volume of | | |
| approximately 130 ml with an omeprazole | | |
| concentration of about 1.5 mg/ml. | | |

FORMULATION 3

| PART A INGREDIENTS (mg) | AMOUNT (mg) |
|---|---|
| Sucrose | 26000 |
| Sodium Bicarbonate | 9400 |
| Cocoa | 1800 |
| Corn Syrup Solids | 6000 |
| Sodium Caseinate | 1000 |
| Soy Lecithin | 150 |
| Sodium Chloride | 35 |
| Tricalcium Phosphate | 20 |
| Dipotassium Phosphate | 12 |
| Silicon Dioxide | 5 |
| Sodium Stearoyl Lactylate | 5 |

| PART B INGREDIENTS | AMOUNT | |
|---|---|---|
| Distilled Water | 100 | ml |
| Omeprazole | 200 | mg |
| COMPOUNDING INSTRUCTIONS | | |
| This formulation is reconstituted at the | | |
| time of use by a pharmacist. Part B is | | |
| mixed first and is then uniformly mixed | | |
| with the components of Part A. A final | | |
| volume of about 130 ml is created having an | | |
| omeprazole concentration of about 1.5 mg/ml. | | |

FORMULATION 4

| PART A INGREDIENTS (mg) | AMOUNT (mg) |
|---|---|
| Sucrose | 26000 |
| Cocoa | 1800 |
| Corn Syrup Solids | 6000 |
| Sodium Caseinate | 1000 |
| Soy Lecithin | 150 |
| Sodium Chloride | 35 |
| Tricalcium Phosphate | 20 |
| Dipotassium Phosphate | 12 |
| Silicon Dioxide | 5 |
| Sodium Stearoyl Lactylate | 5 |

| PART B INGREDIENTS | AMOUNT | |
|---|---|---|
| Distilled Water | 100 | ml |
| Sodium Bicarbonate | 8400 | mg |
| Omeprazole | 200 | mg |
| COMPOUNDING INSTRUCTIONS | | |
| This formulation is reconstituted at the | | |
| time of use by a pharmacist. Part B is | | |
| mixed first and is then uniformly mixed | | |
| with the components of Part A. A final | | |
| volume of about 130 ml is created having an | | |
| omeprazole concentration of about 1.5 mg/ml. | | |

In all four of the above formulations, lansoprazole or other proton pump inhibitor can be substituted for omeprazole in equipotent amounts. For example, 300 mg of lansoprazole may be substituted for the 200 mg of omeprazole. Additionally, aspartame can be substituted for sucrose, and the following other ingredients can be employed as carriers, adjuvants and excipients: maltodextrin, vanilla, carrageenan, mono and diglycerides, and lactated monoglycerides. One skilled in the art will appreciate that not all of the ingredients are necessary to create a Choco-Base™ formulation that is safe and effective.

Omeprazole powder or enteric-coated granules can be used in each formulation. If the enteric-coated granules are used, the coating is either dissolved by the aqueous diluent or inactivated by trituration in the compounding process.

Applicant additionally analyzed the effects of a lansoprazole Choco-Base™ formulation on gastric pH using a pH meter (Fisher Scientific) in one adult patient versus lansoprazole alone. The patient was first given a 30 mg oral capsule of lansoprazole (Prevacid®), and the patient's gastric pH was measured at 0, 4, 8, 12, and 16 hours post dose. The results are illustrated in FIG. 4.

The ChocoBase product was compounded according to Formulation 1 above, except 300 mg of lansoprazole was used instead of omeprazole. A dose of 30 mg lansoprazole Choco-Base™ was orally administered at hour 18 post lansoprazole alone. Gastric pH was measured using a pH meter at hours 18, 19, 24, 28, 32, 36, 40, 48, 52, and 56 post lansoprazole alone dose.

FIG. 4 illustrates the lansoprazole/cocoa combination resulted in higher pHₛ at hours 19-56 than lansoprazole alone at hours 4-18. Therefore, the combination of the lansoprazole with chocolate enhanced the pharmacologic activity of the lansoprazole. The results establish that the sodium bicarbonate as well as chocolate flavoring and calcium were all able to stimulate the activation of the proton pumps, perhaps due to the release of gastrin. Proton pump inhibiting agents work by functionally inhibiting the proton pump and effectively block activated proton pumps (primarily those inserted into the secretory canalicular membrane). By further administering the proton pump inhibitor with one of these activators or enhancers, there is a synchronization of activation of the proton pump with the absorption and subsequent parietal cell concentrations of the proton pump inhibitor. As illustrated in FIG. 4, this combination produced a much longer pharmacologic effect than when the proton pump inhibitor was administered alone.

### Example VI

Combination Tablet Delivering Bolus And Time-Released Doses of Proton Pump Inhibitor

Tablets were compounded using known methods by forming an inner core of 10 mg omeprazole powder mixed with 750 mg sodium bicarbonate, and an outer core of 10 mg omeprazole enteric-coated granules mixed with known binders and excipients. Upon ingestion of the whole tablet, the tablet dissolves and the inner core is dispersed in the stomach where it is absorbed for immediate therapeutic effect. The enteric-coated granules are later absorbed in the duodenum to provide symptomatic relief later in the dosing cycle. This tablet is particularly useful in patients who experience breakthrough gastritis between conventional doses, such as while sleeping or in the early morning hours.

### Example VII

Therapeutic Application.

Patients were evaluable if they met the following criteria: had two or more risk factors for SRMD (mechanical ventilation, head injury, severe burn, sepsis, multiple trauma, adult respiratory distress syndrome, major surgery, acute renal failure, multiple operative procedures, coagulotherapy, significant hyportension, acid-base disorder, and hepatic failure), gastric pH of ≦4 prior to study entry, and no concomitant prophylaxis for SRMD.

The omeprazole solution was prepared by mixing 10 ml of 8.4% sodium bicarbonate with the contents of a 20 mg capsule of omeprazole (Merck & Co. Inc., West Point, Pa.) to yield a solution having a final omeprazole concentration of 2 mg/ml.

Nasogastric (ng) tubes were placed in the patients and an omeprazole dosage protocol of buffered 40 mg omeprazole solution (2 mg omeprazole/1 ml NaHCO₃—8.4%) followed by 40 mg of the same buffered omeprazole solution in eight hours, then 20 mg of the same buffered omeprazole solution per day, for five days. After each buffered omeprazole solution administration, nasogastric suction was turned off for thirty minutes.

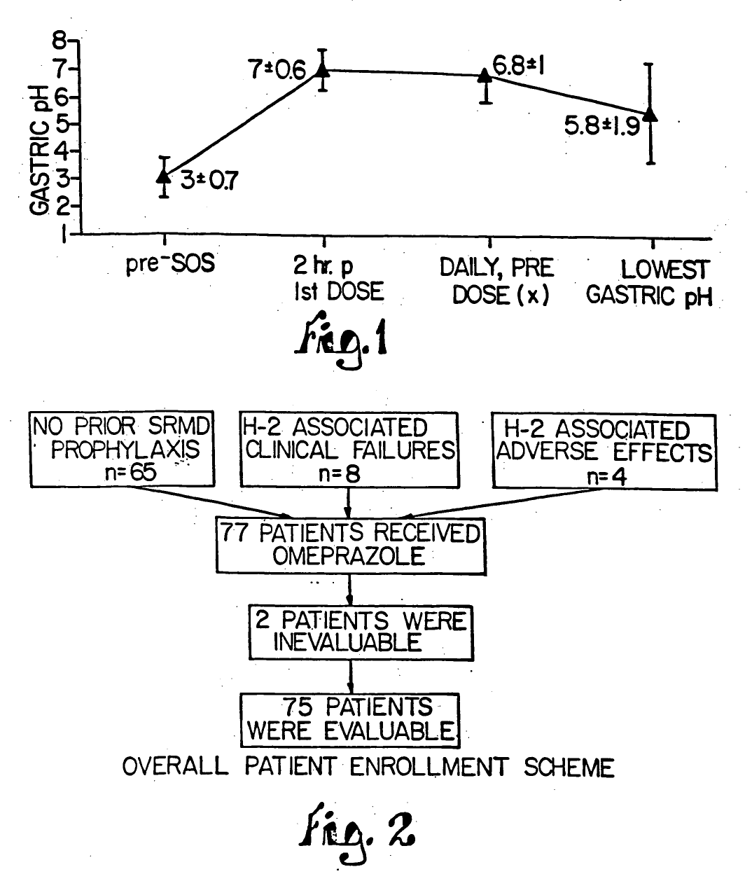

Eleven patients were evaluable. All patients were mechanically ventilated. Two hours after the initial 40 mg dose of buffered omeprazole solution, all patients had an increase in gastric pH to greater than eight as shown in FIG. 1. Ten of the eleven patients maintained a gastric pH of greater than or equal to four when administered 20 mg omeprazole solution. One patient required 40 mg omeprazole solution per day (closed head injury, five total risk factors for SRMD). Two patients were changed to omeprazole solution after having developed clinically significant upper gastrointestinal bleeding while receiving conventional intravenous H₂-antagonists. Bleeding subsided in both cases after twenty-four hours. Clinically significant upper gastrointestinal bleeding did not occur in the other nine patients. Overall mortality was 27%, mortality attributable to upper gastrointestinal bleeding was 0%. Pneumonia developed in one patient after initiating omeprazole therapy and was present upon the initiation of omeprazole therapy in another patient. The mean length of prophylaxis was five days.

A pharmacoeconomic analysis revealed a difference in the total cost of care for the prophylaxis of SRMD:
ranitidine (Zantac®) continuous infusion intravenously (150 mg/24 hours)×five days $125.50;
cimetidine (Tagamet®) continuous infusion intravenously (900 mg/24 hours)×five days $109.61;
sucralfate one g slurry four times a day per (ng) tube×five days $73.00; and
buffered omeprazole solution regimen per (ng) tube×five days $65.70.

This example illustrates the efficacy of the buffered omeprazole solution of the present invention based on the increase in gastric pH, safety and cost of the buffered omeprazole solution as a method for SRMD prophylaxis.

### Example VIII

Effect on pH.

Experiments were carried out in order to determine the effect of the omeprazole solution (2 mg omeprazole/1 ml NaHCO₃—8.4%) administration on the accuracy of subsequent pH measurements through a nasogastric tube.

After preparing a total of 40 mg of buffered omeprazole solution, in the manner of Example VII, doses were administered into the stomach, usually through a nasogastric (ng) tube. Nasogastric tubes from nine different institutions were gathered for an evaluation. Artificial gastric fluid (gf) was prepared according to the USP. pH recordings were made in triplicate using a Microcomputer Portable pH meter model 6007 (Jenco Electronics Ltd., Taipei, Taiwan).

First, the terminal portion (tp) of the nasogastric tubes was placed into a glass beaker containing the gastric fluid. A 5 ml aliquot of gastric fluid was aspirated through each tube and the pH recorded; this was called the “pre-omeprazole solution/suspension measurement.” Second, the terminal portion (tp) of each of the nasogastric tubes was removed from the beaker of gastric fluid and placed into an empty beaker. Twenty (20) mg of omeprazole solution was delivered through each of the nasogastric tubes and flushed with 10 ml of tap water. The terminal portion (tp) of each of the nasogastric tubes was placed back into the gastric fluid. After a one hour incubation, a 5 ml aliquot of gastric fluid was aspirated through each nasogastric tube and the pH recorded; this was called the “after first dose SOS [Simplified Omeprazole Solution] measurement.” Third, after an additional hour had passed, the second step was repeated; this was called the “after second dose SOS [Simplified Omeprazole Solution] measurement.” In addition to the pre-omeprazole measurement, the pH of the gastric fluid was checked in triplicate after the second and third steps. A change in the pH measurements of ±0.3 units was considered significant. The Friedman test was used to compare the results. The Friedman test is a two way analysis of variance which is used when more than two related samples are of interest, as in repeated measurements.

The results of these experiments are outlined in Table 3.

TABLE 3

| | ng1 | ng2 | ng3 | ng4 | ng5 | ng6 | ng7 | ng8 | ng9 |
|---|---|---|---|---|---|---|---|---|---|
| [1] gf | 1.3 | 1.3 | 1.3 | 1.3 | 1.3 | 1.3 | 1.3 | 1.3 | 1.3 |
| Pre SOS | | | | | | | | | |
| [2] gf p | 1.3 | 1.3 | 1.3 | 1.3 | 1.3 | 1.3 | 1.3 | 1.3 | 1.3 |
| 1^{st} dose | | | | | | | | | |
| 1.3←check of gf pH | | | | | | | | | |
| [3] gf p | 1.3 | 1.3 | 1.4 | 1.4 | 1.4 | 1.3 | 1.4 | 1.3 | 1.3 |
| 2^{nd} Dose | | | | | | | | | |

| | |
|---|---|
| 1.3←check of gf pH | SOS pH = 9.0 |

Table 3 illustrates the results of the pH measurements that were taken during the course of the experiment. These results illustrate that there were no statistically significant latent effects of omeprazole solution administration (per nasogastric tube) on the accuracy of subsequent pH measurements obtained through the same nasogastric tube.

### Example IX

Efficacy of Buffered Omeprazole Solution in Ventilated Patients.

Experiments were performed in order to determine the efficacy, safety, and cost of buffered omeprazole solution in mechanically ventilated critically ill patients who have at least one additional risk factor for stress-related mucosal damage.

Patients: Seventy-five adult, mechanically ventilated patients with at least one additional risk factor for stress-related mucosal damage.

Interventions: Patients received 20 ml omeprazole solution (prepared as per Example VII and containing 40 mg of omeprazole) initially, followed by a second 20 ml dose six to eight hours later, then 10 ml (20 mg) daily. Omeprazole solution according to the present invention was administered through a nasogastric tube, followed by 5 -10 ml of tap water. The nasogastric tube was clamped for one to two hours after each administration.

Measurements and Main Results: The primary outcome measure was clinically significant gastrointestinal bleeding determined by endoscopic evaluation, nasogastric aspirate examination, or heme-positive coffee ground material that did not clear with lavage and was associated with a five percent decrease in hematocrit. Secondary efficacy measures were gastric pH measured four hours after omeprazole was first administered, mean gastric pH after omeprazole was started, and the lowest gastric pH during omeprazole therapy. Safety-related outcomes included the incidence of adverse events and the incidence of pneumonia. No patient experienced clinically significant upper gastrointestinal bleeding after receiving omeprazole suspension. The four-hour post omeprazole gastric pH was 7.1 (mean), the mean gastric pH after starting omeprazole was 6.8 (mean) and the lowest pH after starting omeprazole was 5.6 (mean). The incidence of pneumonia was twelve percent. No patient in this high-risk population experienced an adverse event or a drug interaction that was attributable to omeprazole.

Conclusions: Omeprazole solution prevented clinically significant upper gastrointestinal bleeding and maintained gastric pH above 5.5 in mechanically ventilated critical care patients without producing toxicity.

Materials and Methods:

The study protocol was approved by the Institutional Review Board for the University of Missouri at Columbia.

Study Population: All adult (>18 years old) patients admitted to the surgical intensive care and burn unit at the University of Missouri Hospital with an intact stomach, a nasogastric tube in place, and an anticipated intensive care unit stay of at least forty-eight hours were considered for inclusion in the study. To be included patients also had to have a gastric pH of <4, had to be mechanically ventilated and have one of the following additional risk factors for a minimum of twenty-four hours after initiation of omeprazole suspension: head injury with altered level of consciousness, extensive bums (>20% Body Surface Area), acute renal failure, acid-base disorder, multiple trauma, coagulopathy, multiple operative procedures, coma, hypotension for longer than one hour or sepsis (see Table 4). Sepsis was defined as the presence of invasive pathogenic organisms or their toxins in blood or tissues resulting in a systematic response that included two or more of the following: temperature greater than 38° C. or less than 36° C., heart rate greater than 90 beats/minute, respiratory rate greater than 20 breaths/minute (or ₚO₂ less than 75 mm Hg), and white blood cell count greater than 12,000 or less than 4,000 cells/mm³ or more than 10 percent bands (Bone, Let's Agree on Terminology: Definitions of Sepsis, CRIT. CARE MED., 19:27 (1991)). Patients in whom H₂-antagonist therapy had failed or who experienced an adverse event while receiving H₂-antagonist therapy were also included.

Patients were excluded from the study if they were receiving azole antifungal agents through the nasogastric tube; were likely to swallow blood (e.g., facial and/or sinus fractures, oral lacerations); had severe thrombocytopenia (platelet count less than 30,000 cells/mm³); were receiving enteral feedings through the nasogastric tube; or had a history of vagotomy, pyloroplasty, or gastroplasty. In addition, patients with a gastric pH above four for forty-eight hours after ICU admission (without prophylaxis) were not eligible for participation. Patients who developed bleeding within the digestive tract that was not stress-related mucosal damage (e.g., endoscopically verified variceal bleeding or Mallory-Weiss tears, oral lesions, nasal tears due to placement of the nasogastric tube) were excluded from the efficacy evaluation and categorized as having non-stress-related mucosal bleeding. The reason for this exclusion is the confounding effect of non-stress-related mucosal bleeding on efficacy-related outcomes, such as the use of nasogastric aspirate inspection to define clinically significant upper gastrointestinal bleeding.

Study Drug Administration: Omeprazole solution was prepared immediately before administration by the patient's nurse using the following instructions: empty the contents of one or two 20 mg omeprazole capsule(s) into an empty 10 ml syringe (with 20 gauge needle in place) from which the plunger has been removed. (Omeprazole delayed-release capsules, Merck & Co., Inc., West Point, Pa.); replace the plunger and uncap the needle; withdraw 10 ml of 8.4% sodium bicarbonate solution or 20 ml if 40 mg given (Abbott Laboratories, North Chicago, Ill.), to create a concentration of 2 mg omeprazole per ml of 8.4% sodium bicarbonate; and allow the enteric coated pellets of omeprazole to completely breakdown, ≈30 minutes (agitation is helpful). The omeprazole in the resultant preparation is partially dissolved and partially suspended. The preparation should have a milky white appearance with fine sediment and should be shaken before administration. The solution was not administered with acidic substances. A high-pressure liquid chromatography study was performed that demonstrated that this preparation of simplified omeprazole suspension maintains >90% potency for seven days at room temperature. This preparation remained free of bacterial and fungal contamination for thirty days when stored at room temperature (See Table 7).

The initial dose of omeprazole solution was 40 mg, followed by a second 40 mg dose six to eight hours later, then a 20 mg daily dose administered at 8:00 AM. Each dose was administered through the nasogastric tube. The nasogastric tube was then flushed with 5-10 ml of tap water and clamped for at least one hour. Omeprazole therapy was continued until there was no longer a need for stress ulcer prophylaxis (usually after the nasogastric tube was removed and the patient was taking water/food by mouth, or after the patient was removed from mechanical ventilation).

Primary Outcome Measures: The primary outcome measure in this study was the rate of clinically significant stress-related mucosal bleeding defined as endoscopic evidence of stress-related mucosal bleeding or bright red blood per nasogastric tube that did not clear after a 5-minute lavage or persistent Gastroccult (SmithKline Diagnostics, Sunnyville, Calif.) positive coffee ground material for four consecutive hours that did not clear with lavage (at least 100 ml) and produced a 5% decrease in hematocrit.

Secondary Outcome Measures: The secondary efficacy measures were gastric pH measured four hours after omeprazole was administered, mean gastric pH after starting omeprazole and lowest gastric pH during omeprazole administration. Gastric pH was measured immediately after aspirating gastric contents through the nasogastric tube. pH paper (pHydrion improved pH papers, Microessential Laboratory, Brooklyn, N.Y.) was used to measure gastric aspirate pH. The pH range of the test strips was 1 to 11, in increments of one pH unit. Gastric pH was measured before the initiation of omeprazole solution therapy, immediately before each dose, and every four hours between doses.

Other secondary outcome measures were incidence of adverse events (including drug interactions) and pneumonia. Any adverse event that developed during the study was recorded. Pneumonia was defined using indicators adapted from the Centers for Disease Prevention and Control definition of nosocomial pneumonia (Garner et al., 1988). According to these criteria, a patient who has pneumonia is one who has rales or dullness to percussion on physical examination of the chest or has a chest radiograph that shows new or progressive infiltrate(s), consolidation, cavitation, or pleural effusion and has at least two of the following present: new purulent sputum or changes in character of the sputum, an organism isolated from blood culture, fever or leukocytosis, or evidence of infection from a protective specimen brush or bronchoalveolar lavage. Patients who met the criteria for pneumonia and were receiving antimicrobial agents for the treatment of pneumonia were included in the pneumonia incidence figure. These criteria were also used as an initial screen before the first dose of study drug was administered to determine if pneumonia was present prior to the start of omeprazole suspension.

Cost of Care Analysis: A pharmacoeconomic evaluation of stress ulcer prophylaxis using omeprazole solution was performed. The evaluation included total drug cost (acquisition and administration), actual costs associated with adverse events (e.g., psychiatry consultation for mental confusion), costs associated with clinically significant upper gastrointestinal bleeding. Total drug cost was calculated by adding the average institutional costs of omeprazole 20 mg capsules, 50 ml sodium bicarbonate vials, and 10 ml syringes with needle; nursing time (drug administration, pH monitoring); pharmacy time (drug preparation); and disposal costs. Costs associated with clinically significant upper gastrointestinal bleeding included endoscopy charges and accompanying consultation fees, procedures required to stop the bleeding (e.g., surgery, hemostatic agents, endoscopic procedures), increased hospital length of stay (as assessed by the attending physician), and cost of drugs used to treat the gastrointestinal bleeding.

Statistical Analysis: The paired t-test (two-tailed) was used to compare gastric pH before and after omeprazole solution administration and to compare gastric pH before omeprazole solution administration with the mean and lowest gastric pH value measured after beginning omeprazole.

Results:

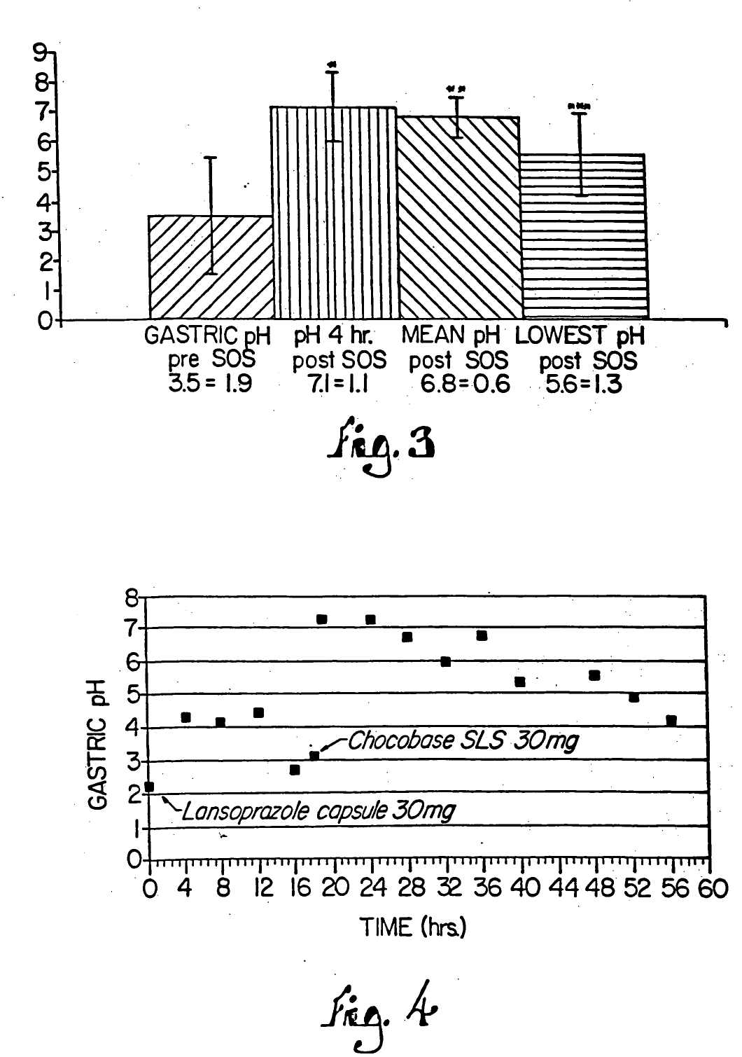

Seventy-seven patients met the inclusion and exclusion criteria and received omeprazole solution (See FIG. 2). Two patients were excluded from the efficacy evaluation because the protocol for omeprazole administration was not followed. In one case, the omeprazole enteric-coated pellets had not completely broken down prior to the administration of the first two doses, which produced an erratic effect on gastric pH. The gastric pH increased to above six as soon as the patient was given a dose of omeprazole solution (in which the enteric coated pellets of omeprazole had been allowed to completely breakdown).

The reason for the second exclusion was that nasogastric suctioning was not turned off after the omeprazole dose was administered. This resulted in a transient effect on gastric pH. The suction was turned off with subsequent omeprazole doses, and control of gastric pH was achieved. Two patients were considered efficacy failures because omeprazole failed to maintain adequate gastric pH control on the standard omeprazole 20 mg/day maintenance dose. When the omeprazole dose was increased to 40 mg/day (40 mg once/day or 20 mg twice/day), gastric pH was maintained above four in both patients. These two patients were included in the safety and efficacy evaluations, including the gastric pH analysis. After the two patients were declared failures, their pH values were no longer followed.

The ages of the remaining seventy-five patients ranged from eighteen to eighty-seven years; forty-two patients were male and thirty-three were female. All patients were mechanically ventilated during the study. Table 4 shows the frequency of risk factors for stress-related bleeding that were exhibited by the patients in this study. The most common risk factors in this population were mechanical ventilation and major surgery. The range of risk factors for any given patient was two to ten, with a mean of 3 (±1) (standard deviation). Five patients enrolled in the study had developed clinically significant bleeding while receiving continuous infusions of ranitidine (150 mg/24 hr) or cimetidine (900 mg/24 hr). In all five cases, the bleeding subsided and the gastric pH rose to above five within thirty-six hours after initiating omeprazole therapy. Three patients were enrolled after having developed two consecutive gastric pH values below three while receiving an H₂-antagonist (in the doses outlined above). In all three cases, gastric pH rose to above five within four hours after omeprazole therapy was initiated. Four other patients were enrolled in this study after experiencing confusion (n=2) or thrombocytopenia (n=2) during H₂-antigens therapy. Within thirty-six hours of switching therapy, these adverse events resolved.

Stress-related Mucosal Bleeding and Mortality: None of the sixty-five patients who received buffered omeprazole solution as their initial prophylaxis against stress-related mucosal bleeding developed overt or clinically significant upper gastrointestinal bleeding. In four of the five patients who had developed upper gastrointestinal bleeding before study entry, bleeding diminished to the presence of occult blood only (Gastroccult-positive) within eighteen hours of starting omeprazole solution; bleeding stopped in all patients within thirty-six hours. The overall mortality rate in this group of critically ill patients was eleven percent. No death was attributable to upper gastrointestinal bleeding or the use of omeprazole solution.

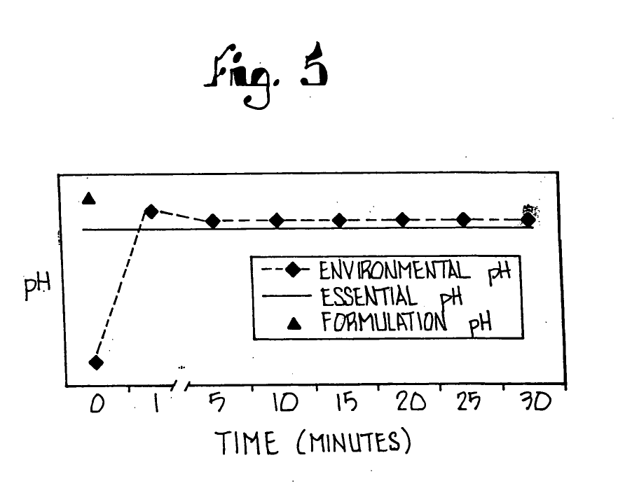

Gastric pH: The mean (+standard deviation) pre-omeprazole gastric pH was 3.5±1.9. Within four hours of omeprazole administration, the gastric pH rose to 7.1±1.1 (See FIG. 3); this difference was significant (p<0.001). The differences between pre-omeprazole gastric pH and the mean and lowest gastric pH measurements during omeprazole administration (6.8±0.6 and 5.6±1.3, respectively) were also statistically significant (p<0.001).

Safety: Omeprazole solution was well tolerated in this group of critically ill patients. Only one patient with sepsis experienced an adverse event that may have been drug-related thrombocytopenia. However, the platelet count continued to fall after omeprazole was stopped. The platelet count then returned to normal despite reinstitution of omeprazole therapy. Of note, one patient on a jet ventilator continuously expelled all liquids placed in her stomach up and out through her mouth, and thus was unable to continue on omeprazole. No clinically significant drug interactions with omeprazole were noted during the study period. As stated above, metabolic alkalosis is a potential concern in patients receiving sodium bicarbonate. However, the amount of sodium bicarbonate in omeprazole solution was small (≈12 mEq/10 ml) and no electrolyte abnormalities were found.

Pneumonia: Pneumonia developed in nine (12%) patients receiving omeprazole solution. Pneumonia was present in an additional five patients before the start of omeprazole therapy.

Pharmacoeconomic evaluation: The average length of treatment was nine days. The cost of care data are listed in Tables 5 and 6. The costs of drug acquisition, preparation, and delivery for some of the traditional agents used in the prophylaxis of stress-related upper gastrointestinal bleeding are listed in Table 5. There were no costs to add from toxicity associated with omeprazole solution. Since two of seventy-five patients required 40 mg of omeprazole solution daily to adequately control gastric pH, the acquisition/preparation cost should reflect this. The additional 20 mg of omeprazole with vehicle adds seven cents per day to the cost of care. Therefore, the daily cost of care for omeprazole solution in the prophylaxis of stress-related mucosal bleeding was $12.60 (See Table 6).

Omeprazole solution is a safe and effective therapy for the prevention of clinically significant stress-related mucosal bleeding in critical care patients. The contribution of many risk factors to stress-related mucosal damage has been challenged recently. All of the patients in this study had at least one risk factor that has clearly been associated with stress-related mucosal damage—mechanical ventilation. Previous trials and data from a recently published study show that stress ulcer prophylaxis is of proven benefit in patients at risk and, therefore, it was thought to be unethical to include a placebo group in this study. No clinically significant upper gastrointestinal bleeding occurred during omeprazole solution therapy. Gastric pH was maintained above 4 on omeprazole 20 mg/day in seventy-three of seventy-five patients. No adverse events or drug interaction associated with omeprazole were encountered.

TABLE 4

| Mech | Major | Multi- | Head | Hypo- | Renal | | Multiple | Acid/ | | Liver | |
|---|---|---|---|---|---|---|---|---|---|---|---|
| Vent | Surgery | trauma | Injury | tension | Failure | Sepsis | Operation | BaseBase ™ | Coma | Failure | Burn |
| 75 | 61 | 35 | 16 | 14 | 14 | 14 | 12 | 10 | 4 | 2 | 2 |

Risk factors present in patients in this study (n = 75)

TABLE 5

| | | Per day |
|---|---|---|
| RANITIDINE (day 1-9) | | |
| Rantidine | 150 mg/24 hr | 6.15 |
| Ancillary Product (1) | Piggyback (60%) | 0.75 |
| Ancillary Product (2) | micro tubing (etc.) | 2.00 |
| Ancillary Product (3) | filter | 0.40 |
| Sterile Prep required | yes | |
| R.N. time ($24/hr) | 20 minutes/day (includes | 8.00 |
| | pH monitoring) | |
| R.Ph. time, hood maint. | 3 minutes ($40/hr) | 2.00 |
| Pump cost | $29/24 hrs × 50%) | 14.50 |
| TOTAL for 9 days | | 304.20 |
| RANITIDINE Cost per day | | 33.80 |
| CIMETIDINE (day 1-9) | | |
| Cimetidine | 900 mg/24 hr | 3.96 |
| Ancillary Product (1) | Piggyback | 1.25 |
| Ancillary Product (2) | micro tubing (etc.) | 2.00 |
| Ancillary Product (3) | filter | 0.40 |
| Sterile Prep required | yes | 8.00 |
| R.N. time ($24/hr) | 20 minutes/day (includes | |
| | pH monitoring) | |
| R.Ph. time, hood maint. | 3 minutes ($40/hr) | 2.00 |
| Pump cost | $29/24 hrs × 50%) | 14.50 |
| TOTAL for 9 days | | 288.99 |
| CIMETIDINE Cost per day | | 32.11 |
| SUCRALFATE (day 1-9) | | |
| Sucralfate | 1 g × 4 | 2.40 |
| Ancillary Product (1) | syringe | 0.20 |
| Sterile Prep required | no | |
| R.N. time ($24/hr) | 30 minutes/day (includes | 12.00 |
| | pH monitoring) | |
| TOTAL for 9 days | | 131.40 |
| SUCRALFATE Cost per day | | 14.60 |

Note:
Does not include the cost of failure and/or adverse effect.
Acquisition, preparation and delivery costs of traditional agents.

TABLE 6
The average length of treatment was 9 days.
Cost of care was calculated from these date

| | | Per Day | Total |
|---|---|---|---|
| OMEPRAZOLE (day 1) | | | |
| Product acquisition cost | 40 mg load × 2 (5.66/dose) | 11.32 | 11.32 |
| Ancillary product | materials for solution | 0.41 | 0.41 |
| | preparation | | |
| Ancillary product | syringe w/needle | 0.20 | 0.40 |
| Sterile preparation | no | | |
| required | 6 minutes | 2.40 | 4.80 |
| SOS preparation time | 21 minutes/day (includes | 8.40 | 8.40 |
| (R.N.) | pH monitoring) | | |
| R.N. time ($24/hr) | | | |
| OMEPRAZOLE (days 2-9) | | | |
| Product acqusition cost | 20 mg per day | 2.80 | 22.65 |
| Ancillary product | materials for solution | 0.41 | 0.82 |
| | preparation | | |
| Ancillary product | syringe w/needle | 0.20 | 1.60 |
| Sterile preparation | no | | |
| required | 6 minutes | 2.40 | 4.80 |
| SOS preparation time | 18 minutes/day (includes | 8.40 | 57.60 |
| (R.N.) | pH monitoring) | | |
| R.N. time ($24/hr) | | | |

2/75 patient require 40 mg simplified omeparzole solution per day
(days 2-9)
No additional cost for adverse effects or for failure
TOTAL
Simplified Omerprazole Solution cost per day
Pharmacoeconomic evaluation of omeprazole cost of care

TABLE 7

| Time | Control | 1 hour | 24 hour | 2 day | 7 day | 14 day |
|---|---|---|---|---|---|---|
| Conc (mg/ml) | 2.01 | 2.07 | 1.94 | 1.96 | 1.97 | 1.98 |

Stability of Simplified Omeprazole Solution at room temperature (25° C.) Values are the mean of three samples

### Example X

Bacteriostatic and Fungistatic Effects of Omeprazole Solution

The antimicrobial or bacteriostatic effects of the omeprazole solution were analyzed by applicant. An omeprazole solution (2 mg/ml of 8.4% sodium bicarbonate) made according to the present invention was stored at room temperature for four weeks and then was analyzed for fungal and bacterial growth. Following four weeks of storage at room temperature, no bacterial or fungal growth was detected.

An omeprazole solution (2 mg/ml of 8.4% sodium bicarbonate) made in accordance with the present invention was stored at room temperature for twelve weeks and then was analyzed for fungal and bacterial growth. After twelve weeks of incubation at room temperature, no fungal or bacterial growth was detected.

The results of these experiments illustrate the bacteriostatic and fungistatic characteristics of the omeprazole solution of the present invention.

### Example XI

A. Bioequivalency Study.

Healthy male and female study participants over the age of 18 will be randomized to receive omeprazole in the following forms:
(A) 20 mg of a liquid formulation of approximately 20 mg omeprazole in 4.8 mEq sodium bicarbonate qs to 10 ml with water;
(B) 20 mg of a liquid formulation of approximately 2 mg omeprazole per 1 ml of 8.4% sodium bicarbonate.
(C) Prilosec® (omeprazole) 20 mg capsule;
(D) Capsule prepared by inserting non-enteric coated omeprazole 20 mg into a #4 empty gelatin capsule (Lilly) uniformly dispersed in 240 mg of sodium bicarbonate powder USP to form an inner capsule. The inner capsule is then inserted into a #00 empty gelatin capsule (Lilly) together with a homogeneous mixture of 600 mg sodium bicarbonate USP and 110 mg pregelatinized starch NF.

After appropriate screening and consent, healthy volunteers will be randomized to receive one of the following four regimens as randomly assigned by Latin Square. Each subject will be crossed to each regimen according to the randomization sequence until all subjects have received all four regimens (with one week separating each regimen).

Regimen A (20 mg omeprazole in 4.8 mEq sodium bicarbonate in 10 ml volume); Regimen B (20 mg omeprazole in 10 ml 8.4% sodium bicarbonate in 10 ml volume); Regimen C (an intact 20 mg omeprazole capsule); Regimen D (Capsule in capsule formulation, see above). For each dose/week, subjects will have an i.v. saline lock placed for blood sampling. For each regimen, blood samples will be taken over 24 hours a total of 16 times (with the last two specimens obtained 12 hours and 24 hours after drug administration).

B. Patient Eligibility

Four healthy females and four healthy males will be consented for the study.

C. Inclusion Criteria

Signed informed consent.

D. Exclusion Criteria

1. Currently taking H₂-receptor antagonist, antacid, or sucralfate.

2. Recent (within 7 days) therapy with lansoprazole, omeprazole, or other proton pump inhibitor.

3. Recent (within 7 days) therapy with warfarin.

4. History of variceal bleeding.

5. History of peptic ulcer disease or currently active G.I. bleed.

6. History of vagotomy or pyloroplasty.

7. Patient has received an investigational drug within 30 days.

8. Treatment with ketoconazole or itraconazole.

9. Patient has an allergy to omeprazole.

E. Pharmocokinetic Evaluation and Statistical Analysis

Blood samples will be centrifuged within 2 hours of collection and the plasma will then separated and frozen at −10° C. (or lower) until assayed. Pharmacokinetic variables will include: time to peak concentration, mean peak concentration, AUC (0-t) and (0-infinity). Analysis of variance will be used to detect statistical difference. Bioavailability will be assessed by the 90% confidence interval of the two one-sided tests on the natural logarithm of AUC.

F. HPLC Analysis

Omeprazole and internal standard (H168/24) will be used. Omeprazole and internal standard will be measured by modification of the procedure described by Amantea and Narang. (Amantea MA, Narang PK. Improved Procedure for Quantification of Omeprazole and Metabolites Using Reversed-Phased High Performance Liquid Chromotography. J. CHROMATOGRAPHY 426; 216-222 (1988)). Briefly, 20 ul of omeprazole 2 mg/ml NaHCO₃ or Choco-Base™ omeprazole suspension and 100 ul of the internal standard are vortexed with 150 ul of carbonate buffer (pH=9.8), 5 ml of dichloroethane, 5 ml of hexane, and 980 ul of sterile water. After the sample is centrifuged, the organic layer is extracted and dried over a nitrogen stream. Each pellet is reconstituted with 150 ul of mobile phase (40% methanol, 52% 0.025 phosphate buffer, 8% acetonitrile, pH=7.4). Of the reconstituted sample, 75 ul is injected onto a C₁₈ 5 U column equilibrated with the same mobile phase at 1.1 ml/min. Under these conditions, omeprazole is eluted at approximately 5 minutes, and the internal standard at approximately 7.5 minutes. The standard curve is linear over the concentration range 0-3 mg/ml (in previous work with SOS), and the between-day coefficient of variation has been <8% at all concentrations. The typical mean R² for the standard curve has been 0.98 in prior work with SOS (omeprazole 2 mg/ml NaHCO₃ 8.4%).

Applicant expects that the above experiments will demonstrate there is more rapid absorption of formulations (a), (b) and (d) as compared to the enteric coated granules of formulation (c). Additionally, applicant expects that although there will be a difference in the rates of absorption among forms (a) through (d), the extent of absorption (as measured by the area under the curve (AUC)) should be similar among the formulations (a) through (d).

### Example XII

Intraveneous Proton Pump Inhibitor in Combination With Oral Parietal Cell Activator

Sixteen (16) normal, healthy male and female study subjects over the age of 18 will be randomized to receive pantoprazole as follows:
(a) 40 mg IV over 15 to 30 minutes in combination with a 20 ml oral dose of sodium bicarbonate 8.4%; and
(b) 40 mg IV over 15 to 30 minutes in combination with a 20 ml oral dose of water.

The subjects will receive a single dose of (a) or (b) above, and will be crossed-over to (a) and (b) in random fashion. Serum concentrations of pantoprazole versus time after administration data will be collected, as well as gastric pH control as measured with an indwelling pH probe.

Further, similar studies are contemplated wherein chocolate or other parietal cell activator is substituted for the parietal cell activator sodium bicarbonate, and other proton pump inhibiting agents are substituted for pantoprazole. The parietal cell activator can be administered either within about 5 minutes before, during or within about 5 minutes after the IV dose of proton pump inhibitor.

Applicant expects that these studies will demonstrate that significantly less IV proton pump inhibitor is required to achieve therapeutic effect when it is given in combination with an oral parietal cell activator.

Additionally, administration kits of IV proton pump inhibitor and oral parietal cell activator can be packaged in many various forms for ease of administration and to optimize packing and shipping the product. Such kits can be in unit dose or multiple dose form.

### Example XIII

Six (6) Month Stability of Omeprazole Suspension.

A suspension was prepared by mixing 8.4% sodium bicarbonate with omeprazole to produce a final concentration of 2 mg/ml to determine the stability of omeprazole solution after 6 months. The resultant preparation was stored in clear glass at room temperature, refrigerated and frozen. Samples were drawn after thorough agitation from the stored preparations at the prescribed times. The samples were then stored at 70° C. Frozen samples remained frozen until they were analyzed. When the collection process was completed, the samples were shipped to a laboratory overnight on dry ice for analysis. Samples were agitated for 30 seconds and sample aliquots were analyzed by HPLC in triplicate according to well known methods. Omeprazole and the internal standard were measured by a modification of the procedure described by Amantea and Narang. (Amantea M A, Narang P K, Improved Procedure For Quantitation Of Omeprazole And Metabolites Using Reverse-Phased High-Performance Liquid Chromatography, J. CHROMATOGRAPHY, 426:216-222 (1988)). Twenty (20) ul of the omeprazole 2 mg/ml NaHCO₃ solution and 100 ul of the internal standard solution were vortexed with 150 ul of carbonate buffer (pH=9.8), 5 ml dichloroethane, 5 ml hexane, and 980 ul of sterile water. The sample was centrifuged and the organic layer was extracted and dried over a nitrogen stream. Each pellet was reconstituted with 150 ul of mobile phase (40% methanol, 52% 0.025 phosphate buffer, 8% acetonitrile, pH=7.4). Of the reconstituted sample, 75 ul were injected onto a C185u column equilibrated with the same mobile phase at 1.1 m/min. Omeprazole was eluted at ˜5 min, and the internal standard at ˜7.5 min. The standard curve was linear over the concentrated range 0-3 mg/ml, and between-day coefficient of variation was <8% at all concentrations. Mean R² for the standard curve was 0.980.

The 6 month sample showed stability at greater than 90% of the original concentration of 2 mg/ml. (i.e., 1.88 mg/ml, 1.94 mg/ml, 1.92 mg/ml).

### Example XIV

Pharmacokinetic and Pharmacodynamic Study of Duodenal or Jejunal Administration Compared to Nasogastric Administration of Omeprazole Suspension in Patients at Risk for Stress Ulcers

Omeprazole suspension administered by the jejunal or duodenal route was compared in a randomized, cross-over fashion with nasogastric administration in patients at risk for stress-related GI bleeding. Eligible for study enrollment were all adult patients (>18 yr.) admitted to the surgical intensive care unit who had recently undergone a major surgical procedure or were posttrauma with an Acute Physiological and Chronic Health Evaluation (APACHE II) score >18. To be included in the study, patients were also required to be mechanically ventilated in addition to having at least one of the following risk factors: head injury with altered level of consciousness; extensive bums (>20% body surface area); acute renal failure; acid-base disorder; multiple traumas; coagulopathy; multiple operative procedures; coma; hypotension for >1 h; or sepsis syndrome. Patients were excluded from participation if they had any of the following characteristics: hypochlorhydria; status of “Do Not Resuscitate”; a history of vagotomy, pyloroplasty, or gastroplasty; an allergy to proton pump inhibitors; active GI bleeding (including variceal bleeding); thrombocytopenia (<30,000/mm³ platelets); active peptic ulcer disease treated within the past year; were likely at risk of swallowing blood (i.e., severe facial trauma, oral lacerations, hemoptysis); currently or during the study receiving ketoconazole or itraconazole or enteral tube feedings; or had received an investigational drug within 30 days, omeprazole or another proton pump inhibitor within 5 days, or warfarin or nonsteroidal anti-inflammatory drugs (NSAIDs), including aspirin, within 24 h. Administration of the study drug was not initiated until the patient had documented gastric pH of <4.0. If 48 h had passed and gastric pH was not <4.0, the patient was excluded from study participation. Patients who were on prior acid reducing therapy for <24 h were allowed to participate after discontinuation of their medication and gastric acidity achieved the study-imposed pH range (gastric pH <4.0). Subjects were not allowed to receive antisecretory agents (e.g., H2RA) during the study. The institutional Review Board for the University of Missouri at Columbia approved the protocol and informed consent was obtained before study enrollment for every subject.

Omeprazole suspension was compounded and stored in amber bottles at 4° C. The omeprazole was prepared by dissolving the contents of two 20-mg capsules (Prilosec®, Astra-Zeneca, Wayne, Pa.) in 20 ml of 8.4% sodium bicarbonate (Abbott Laboratories, North Chicago, Ill.) with gentle shaking to assure adequate mixing. The sodium bicarbonate dissolves the enteric-coated beads leaving “free omeprazole” in the suspension.

A nasogastric tube and needle catheter jejunostomy or duodenal tube was placed before study initiation. Placement of the nasogastric tube was confirmed by x-ray and aspiration of gastric contents for pH confirmation. The jejunostomy and duodenal tubes were placed by standard surgical technique and positioning was confirmed by x-ray. On study day 1, when gastric pH decreased to <4, the patients were randomized to receive a single 40 mg dose of omeprazole suspension by either nasogastric tube or jejunal/duodenal administration. When gastric pH subsequently dropped again to <4 (>24 h in all patients), each patient was crossed-over to the other administration route followed by a single 40 mg dose of omeprazole suspension. All patients received the cross-over dose 72 h after the first day and after the pH had dropped to <4. After omeprazole administration, the nasogastric or duodenal/jejunal tube was flushed with 10 ml of water and clamped for 1-2 h. A Latin square cross-over design was used.

A total of 60 ml of blood was collected in 2.5 ml aliquots over a period of 24 h to establish the absorption and pharmacokinetic parameters of omeprazole as administered by the different enteral routes. Blood samples were obtained immediately before each dose of drug and at 3, 5, 10, 20, 30, 60, 120, 240, 480, 720, 960, and 1,440 min after drug administration. All samples were collected in red-top tubes (Vacutainer®, Becton-Dickinson, Franklin Lakes, N.J.), allowed to clot for 30 min at room temperature, and centrifuged for 10 minutes at 1,000 g. The resulting sera was removed and immediately frozen at −70° C. until analysis. The study was conducted for approximately 4 days per patient.

Continuous monitoring of gastric acidity (pH) occurred throughout the study period for all patients who received omeprazole suspension. Continuous gastric pH readings were measured with a Zinetics probe (Zinetics Medical, Salt Lake City, Utah).

Omeprazole plasma concentrations were determined by modification of a previously published high-performance liquid chromatography assay. The range of linearity for the assay was 25-1,000 ng/ml for serum. The lower limits of detection were 10 ng/ml. Coefficients of variation (R²) for the omeprazole assay over the standard curve concentrations were >0.99 for the entire study. Intra- and interassay coefficients of variation were consistently <8.5% at concentrations included in the linearity range.

The serum omeprazole concentration-time data were analyzed via WinNonlin Software, Standard Edition, Version 1.5 (Scientific Consulting, Cary, N.C.). First dose pharmacokinetic parameters including half-life (T_{1/2}), maximum serum-concentration (Cₘₐₓ), time to maximum serum concentration (Tₘₐₓ), drug clearance (C1ₛₛ/F) were estimated using a noncompartmental extravascular dose model. Area under the serum-concentration time curve (AUC) was determined by trapezoidal rule and was extrapolated to 24 h (AUCO₀₋₂₄) and to infinity (AUC₀₋₀₀), using the fitted values of the final plasma concentration time curves.

Demographic, pH, and pharmacokinetic data are reported as the mean ±SD as well as the range for respective values when appropriate. The pharmacodynamic relationship between various pharmacokinetic parameters, including clearance (C1 and AUC, were compared to mean pH values obtained for each respective administration route and analyzed by linear regression. Omeprazole concentrations-time data, graphical representation, and statistical analysis were performed with Prism software (GraphPad, Chicago, Ill.). Ap value of <0.05 was considered significant for all statistical analyses.

Omeprazole absorption and pharmacokinetic analyses were performed in nine critically ill surgical patients (five men and four women). The administration was well tolerated without any apparent adverse events. The mean (±SD) age, weight, and creatinine clearance of these patients were 33±11 yr (range, 23-56 yr), 78±19 kg (range, 59-124 kg), and 95±24.0 ml/min (range, 35-120 ml/min), respectively. No patients had demonstrated liver disease by either clinical or laboratory evidence of hepatic dysfunction. All nine patients received omeprazole via nasogastric administration, compared with seven and two patients who were also randomized to receive the drug via the jejunal or duodenal route, respectively. Pharmacokinetic parameters for both groups are shown in Table 8. The mean plasma concentration-time curves after 40 mg of omeprazole suspension administered via the nasogastric and jejunal/duodenal routes produced a biphasic curve with the higher peak serum concentrations resulting from the jejunal/duodenal group compared to nasogastric administration (1.833±0.416,μg/m vs. 0.970±0.436 μg/ml, p=0.006). Omeprazole absorption was also significantly slower by comparison of time to maximum concentration (Tₘₐₓ) when administered by nasogastric tube vs. jejunal/duodenal administration (108.3±42.0 vs. 12.1±7.9 min. p<0.0001). Other mean pharmacokinetic parameters (t_{1/2}, C1ₛₛ, AUC₀₋₂₄, AUC₀₋₀₀) were not statistically different between the two groups, although there was a trend toward a shorter half-life for patients who received drug via the jejunal/duodenal route.

The mean baseline pH was 1.63±0.89 for the jejunal/duodenal group and 2.12±0.67 for the nasogastric group (p=0.26). Mean intragastric pH values rose to >4 1 h after omeprazole administration and remained >4 for the entire 24-h study period in both groups. When comparing the mean pH data (nasogastric (6.32±1.04) vs. jejunal/duodenal (5.57±1.15), p=0.015) nasogastric administration maintained higher gastric pH values throughout the study with fewer incidences of pH values <4.0 overall.

TABLE 8
Pharmacokinetic Parameters of Omeprazole Suspension

| | Nasogastric | Jejunal/Duodenal | p |
|---|---|---|---|
| Variable | (N = 9) | (N = 9) | Value |
| AUC₀₋₂₄ | 373.3 ± 256.2 | 375.3 ± 340.1 | 0.99 |
| AUC_{0-∞} | 415.1 ± 291.8 | 396.7 ± 388.1 | 0.91 |
| Tₘₐₓ (min) | 108.3 ± 42.0 | 12.1 ± 7.9 | <0.001 |
| T_{1/2} (min) | 250.7 ± 100.0 | 162.9 ± 138.9 | 0.14 |
| C1/F | 0.144 ± 0.098 | 0.199 ± 0.137 | 0.34 |
| Cₘₐₓ (μg/ml) | 0.970 ± 0.436 | 1.833 ± 0.416 | 0.0006 |

Data expressed as mean ± SD.
p < 0.05 considered statistically significant.
AUC₀₋₂₄ = area under the curve from 0 to 24 h;
AUC_{0-∞} = Area under the curve from 0 h to infinity;
Tₘₐₓ = time to maximum serum concentration;
T_{1/2} = half life;
C1/F = drug clearance;
Cₘₐₓ = maximum serum concentration.

In summary, nasogastric administration of SOS resulted in lower maximum mean ±SD serum concentrations compared to jejunal/duodenal dosing (0.970±0.436 vs. 1.833±0.416 μg/ml, p=0.006). SOS absorption was significantly slower when administered via nasogastric tube (108.3±42.0 vs. 12.1±7.9 min, p<0.001). However, all routes of administration resulted in similar SOS area under the serum concentration-time curves (AUC₀₋₀₀) (415.1±291.8 vs. 396.7±388.1 μg □h/ml, p=0.91). Mean intragastric pH values remained >4 at 1 h after SOS administration and remained >4 for the entire 24-h study (nasogastric (6.32±1.04) vs. jejunal/duodenal (5.57±1.15), p=0.015), regardless of administration route.

### Example XV

Simplified Omeprazole Suspension (SOS) Pharmacokinetic/Pharmacodynamic Study in Patients at Risk for Stress-Related Mucosal Damage (SRMD).

A. Protocol

Hospitalized patients who were at risk of stress-related mucosal damage (SRMD) were enrolled in this study to evaluate the serum concentration vs. time profile and intragastric pH changes accompanying a single dose of omeprazole 40 mg in 20 mEq sodium bicarbonate suspension. Patients at risk for SRMD were considered eligible and received no prior treatment with omeprazole (within 5 days). Informed Consent was obtained. A nasogastric tube (with a pH probe—incorporated in the tip—GraphProbe ZineticsMedical) was placed in the stomach by standard means. Patients received a dose of SOS (40 mg omeprazole in 20 mL 8.4% sodium bicarbonate) after the gastric pH dropped below 4. Serum concentrations of omeprazole were drawn at baseline, 3, 5, 10, 15, 20, 30, 45, minutes and at 1, 2, 4, 8, 12, and 24 hours post administration. Gastric pH tracings were made using the ZineticsMedical GraphProbe and the DataLogger from Sandhill scientific.

Serum was ultracentrifuged and stored at −70° C. and sent as a single batch to David Flockhart MD, PhD at Georgetown University Medical Center for HPLC (High Pressure Liquid Chromatography) measurement.

B. Results

The omeprazole plasma concentrations for 17 subjects are provided below in Table Nos. 12, 13, 14, and 15. Below is also a summary the pharmacokinetic and pharmacodynamic findings.

1. Pharmacokinetic

Absorption: Absorption was rapid as indicated by the appearance of omeprazole in serum at <10 minutes in many subjects.

Tmax: The C max (maximum serum concentration) was also rapidly attained when compared to the enteric-coated granules. The C max in most every patient appearing before 1 hour (Tmax).

AUC: The absorption of the omeprazole did not appear to be significantly decreased when compared to omeprazole in the enteric-coated form as measured by Area Under the Curve (AUC).

2. Pharmacodynamic

The gastric pH control appeared to be very rapid and sustained at an unusually high pH for a first dose of omeprazole.

TABLE 9
Omeprazole Concentrations Over time for Patient Nos. 1-5 (μg/ml)

| | | Patient #1 | Patient #2 | Patient #3 | Patient #4 | Patient #5 |
|---|---|---|---|---|---|---|
| | | [Omeprazole] | [Omeprazole] | [Omeprazole] | [Omeprazole] | [Omeprazole] |
| Time | | μg/ml plasma | μg/ml plasma | μg/ml plasma | μg/ml plasma | μg/ml plasma |
| 1 | min. | ND | ND | ND | ND | ND |
| 3 | min. | ND | 0.155 | 0.149 | 0.02 | ND |
| 5 | min. | 0.201 | 0.44 | 0.165 | 0.148 | 0.1 |
| 10 | min. | 0.322 | 0.551 | 0.233 | 0.34 | 0.278 |
| 15 | min. | ND | 0.587 | 0.261 | 0.44 | 0.413 |
| 20 | min. | 0.381 | 1.01 | 0.382 | 0.554 | 0.537 |
| 30 | min. | 0.445 | 1.33 | 0.386 | 0.718 | 0.628 |
| 45 | min. | 0.658 | 1.46 | 0.445 | 0.89 | 0.68 |
| 1 | hr. | 0.755 | 1.24 | 0.501 | 0.893 | 0.749 |
| 2 | hrs. | 0.911 | 0.894 | 0.715 | 0.695 | 0.763 |
| 4 | hrs. | 0.976 | 0.13 | 0.463 | ND | 0.622 |
| 8 | hrs. | 0.78 | 0.05 | 0.305 | ND | 0.319 |
| 12 | hrs. | 0.303 | ND | 0.293 | ND | 0.133 |
| 18 | hrs. | ND | ND | ND | ND | ND |
| 24 | hrs. | 0.218 | ND | 0.215 | ND | ND |

TABLE 10
Omeprazole Concentrations Over time for Patient Nos. 6-10 (μg/ml)

| | | Patient #6 | Patient #7 | Patient #8 | Patient #9 | Patient #10 |
|---|---|---|---|---|---|---|
| | | [Omeprazole] | [Omeprazole] | [Omeprazole] | [Omeprazole] | [Omeprazole] |
| Time | | μg/ml plasma | μg/ml plasma | μg/ml plasma | μg/ml plasma | μg/ml plasma |
| 1 | min. | ND | ND | ND | ND | ND |
| 3 | min. | ND | ND | ND | ND | 0.041 |
| 5 | min. | ND | 0.756 | 0.291 | 0.044 | 0.058 |
| 10 | min. | 0.067 | 1.15 | 0.316 | 0.0525 | 0.117 |
| 15 | min. | 0.072 | 0.95 | 0.34 | 0.073 | 0.192 |
| 20 | min. | 0.05 | ND | 0.44 | 0.096 | 0.213 |
| 30 | min. | 0.0925 | ND | 0.66 | 0.152 | 0.237 |
| 45 | min. | 0.095 | ND | 0.437 | 0.186 | 0.234 |
| 1 | hr. | 0.058 | 0.623 | 0.386 | 0.24 | 0.263 |
| 1 | hr. 15 min. | ND | 0.61 | ND | ND | ND |
| 2 | hrs. | 0.012 | 0.177 | 0.153 | 0.406 | 0.221 |
| 4 | hrs. | ND | 0.107 | 0.044 | 0.865 | 0.391 |
| 8 | hrs. | ND | ND | ND | 0.303 | 0.164 |
| 12 | hrs. | ND | ND | ND | 0.168 | 0.055 |
| 18 | hrs. | ND | ND | ND | ND | ND |
| 24 | hrs. | ND | ND | ND | 0.108 | ND |

TABLE 11
Omeprazole Concentrations Over time for Patient Nos. 11-15 (μg/ml)

| | | Patient #11 | Patient #12 | Patient #13 | Patient #14 | Patient #15 |
|---|---|---|---|---|---|---|
| | | [Omeprazole] | [Omeprazole] | [Omeprazole] | [Omeprazole] | [Omeprazole] |
| Time | | μg/ml plasma | μg/ml plasma | μg/ml plasma | μg/ml plasma | μg/ml plasma |
| 1 | min. | ND | ND | ND | ND | ND |
| 3 | min. | 0.0275 | ND | ND | ND | ND |
| 5 | min. | 0.0735 | ND | ND | ND | 0.1075 |
| | | | | | | (or 20 min.) |
| 10 | min. | 0.131 | ND | 1.12 | 0.131 | 0.155 |
| 15 | min. | 0.154 | ND | 1.08 | 0.161 | 0.176 |
| 17 | min. | ND | ND | ND | ND | ND |
| 20 | min. | 0.177 | 0.012 | 1.04 | 0.187 | ND |
| | | | | | | (or 5 min.) |
| 30 | min. | 0.388 | 0.025 | 0.865 | 0.224 | 0.184 |
| 45 | min. | 0.526 | 0.046 | 0.841 | 0.269 | 0.196 |
| 1 | hr. | 0.486 | 0.077 | 0.896 | 0.276 | 0.155 |
| 2 | hrs. | 0.458 | 0.128 | 0.504 | 0.343 | 0.17 |
| 4 | hrs. | 0.466 | 0.17 | 0.278 | 0.435 | 0.139 |
| 8 | hrs. | 0.232 | 0.148 | 0.145 | 0.204 | ND |
| 12 | hrs. | 0.093 | 0.052 | ND | 0.131 | ND |
| 18 | hrs. | ND | ND | ND | ND | ND |
| 24 | hrs. | ND | ND | ND | ND | ND |

TABLE 12
Omeprazole Concentrations Over time
for Patient Nos. 16-17 (μg/ml)

| | | Patient #16 | Patient #17 |
|---|---|---|---|
| | | [Omeprazole] | [Omeprazole] |
| Time | | μg/ml plasma | μg/ml plasma |
| 1 | min. | ND | ND |
| 3 | min. | ND | ND |
| 5 | min. | ND | ND |
| 10 | min. | ND | 0.504 |
| 15 | min. | ND | 0.6932 |
| 20 | min. | ND | 0.765 |
| 30 | min. | 0.076 | 0.777 |
| 45 | min. | 0.186 | 0.645 |
| 1 | hr. | 0.242 | 0.547 |
| 2 | hrs. | 0.193 | 0.508 |
| 4 | hrs. | ND | ND |
| 8 | hrs. | ND | ND |
| 12 | hrs. | ND | ND |
| 18 | hrs. | ND | ND |
| 24 | hrs. | ND | ND |

### Example XVI

A Comparison of the Pharmacokinetics and Pharmacodynamics of Omeprazole Delivered Orally with Different Doses of Antiacid in Fasted Subjects

A. Administration of Test Articles

Test articles were administered to each subject according to the following schedule:
Period 1: 1 antacid tablet (30 mEq of 1 part sodium bicarbonate to 3 parts calcium carbonate) plus 40 mg omeprazole powder was administered in the fasted state with 60 mL (2 oz.) water.
Period 2: A solution/suspension of omeprazole 40 mg and 20 mEq of sodium bicarbonate (total volume 20 mL in an amber bottle) was administered to the subject. Immediately (within 30 seconds) after administration, the bottle was rinsed with a small amount of water, which was also administered to the subject. The rinse step was repeated and the subject was given a total of 100 mL of water after the administration of the 20 mL of the omeprazole/sodium bicarbonate solution/suspension.
Period 3: 1 capsule of Prilosec (40 mg of enteric-coated omeprazole alone) in the fasted state with 120 mL water.
Period 5: 1 antacid tablet (30 mEq of 1 part sodium bicarbonate to 1 part calcium carbonate) plus 40 mg omeprazole powder was administered in the fasted state with 120 ml water.
   B. Treatment Periods

Only 1 day (Day 1) was required in the clinic. Subjects fasted for at least 10 hours overnight in the clinic prior to initiating pH monitoring; they were allowed water ad libitum until 1 hour prior to dose administration.

Each subject receiving 40 mg of omeprazole powder was administered the drug product by site staff directly onto the dorsal mid-tongue. Immediately thereafter, subjects were administered one or two chewable antacid tablets and began chewing. Each subject continued to chew the tablet(s), while mixing it with the omeprazole powder, carefully avoiding swallowing the powder immediately. One minute after initiating chewing (and after completely swallowing the test articles), each subject drank 60-120 mL of water rising the oral cavity before swallowing. No additional water was allowed until after the 6-hour postdose pH and blood samples were taken. Water was allowed ad libitum. For pharmacokinetic/pharmacodynamic sampling, zero time was the time that chewing is initiated.

C. Inclusion Criteria

Subjects were included in the trial if they met all of the following:
1. Were non-Asian males from 18 to 45 years of age.
2. Were within the ranges of about 20% of ideal body weight.
3. Were in good health on the basis of history, physical examination, and laboratory values.
4. Had not used any form of tobacco (e.g., smoking, chewing) for the last year.
5. Tolerated installation of nasogastric pH probe for at least 5 minutes.
6. Had a basal gastric pH at each trial visit of less than 2.5.
   D. Exclusion Criteria

Subjects were excluded from the trial if they met any of the following:
1. Had a significant history of/or concurrent gastrointestinal disease or condition, such as GERD, heartburn, reflux esophagitis, peptic ulcer disease (gastric or duodenal), or a family history of peptic ulcer disease, gastric surgery (e.g., vagotomy, pyloroplasty).
2. Had any significant medical history or concurrent illness, such as respiratory, allergic, psychiatric, neurological, renal, hepatic, cardiovascular, metabolic, or endocrine condition, or any other medical condition which the investigator or medical monitor considered sufficiently serious to interfere with the conduct, completion, or results of the trial, or constituted an unacceptable risk to the subject.
3. Had a history of significant drug allergy.
4. Known hypersensitivity to any of the ingredients in the test articles.
5. Had a positive urine test of alcohol or other drugs at any trial visit.
6. Had taken any gastric antisecretory drugs, e.g., H2 antagonists or PPIs, or antacids (including OTC medications) within 14 days prior to Period 1 or during the trial.
7. Had taken xanthine-containing foods or beverages (e.g., coffee, tea, chocolate) within 48 hours of entering the clinic for each trial period.
8. Had ingested grapefruit juice within 7 days of dose administration in any trial period.
9. Had donated blood within 90 days of entering the trial.
10. Had been treated with any investigational drug or therapy, or participated in a clinical trial in the 90 days prior to entering the trial.
11. Had any condition which could have interferes with assessments, posed additional risks in administration of the trial drug to the subject, or precluded completion of the trial, including a history of noncompliance, alcoholism, or drug abuse.
12. Had any laboratory test results deviating from the normal reference ranges established by the local laboratory by more then 20% that the investigator judged to be of possible clinical significance.
13. Evidence of infection with HIV.
14. Known carrier of hepatitis B surface antigen.
15. Known carrier of hepatitis C antibody.
   E. Omeprazole Pharmacokinetics

Blood samples (10 mL) for measurement of plasma omeprazole were taken within 30 minutes prior to each dosing, and at 5, 10, 15, 30, 45, 60, 90, 120, 180, 240, 300, and 360 minutes (6 hours) after dosing. These samples were taken at the same time as the gastric pH was being recorded. Plasma omeprazole was measured using a previously validated LC-MSMS assay. Zero time was the time that the subject first chewed a table formulation, swallowed a capsule, or first swallowed a liquid formulation of test article.

F. Test Article Evaluation (Day 1)

On Day 1, after a greater than or less than 10 hour fast, pH recordings of the gastric fluid began in the morning for 1 hour prior to dosing. The pH monitoring continued for 6 hours postdose.

G. Pharmacokinetic Analysis of Omeprazole

The following pharmacokinetic parameters were evaluated:
Omeprazole plasma concentration at each sampling time.
Peak omeprazole plasma concentration (Cₘₐₓ) and time to peak plasma concentration (Tₘₐₓ) obtained directly from the data without interpolation.
Terminal elimination rate constant (kₑₗ) determined from a log-linear regression analysis of the terminal plasma omeprazole concentrations.
Terminal elimination half-life (t_{1/2}) calculated as 0.693/kₑₗ.
Area under the omeprazole plasma concentration-time curve from time zero to time “t” (AUC₀₋ₜ), calculated using the trapezodial rule with the plasma concentration at time “t” being the last measurable concentration.
Area under the omeprazole plasma concentration-time curve from time zero to time infinity (AUC_{0-inf}), calculated as AUC₀₋ₜ+Cₜ/kₑₗ, where Cₜ is the last measurable plasma concentration and kₑₗ is the terminal elimination rate constant defined above.
   H. Onset, Duration, and Magnitude of Effects

Onset of action was defined as the earliest time that the value with active treatment was significantly different from the corresponding baseline value. The baseline value for each subject was the mean of values from the twelve 5-minute baseline periods.

Duration of action was the latest time that the value with active treatment was significantly different from the corresponding baseline value.

Magnitude of effect was evaluated for each 5-minute postdosing interval as well as for the postdosing intervals 0-360 minutes.

I. Description

The chewable antacid tablets were produced by Murty Pharmaceuticals, Inc. (518 Codell Drive, Lexington, Ky. 40509-1016) and contained sodium bicarbonate and calcium carbonate, as well as common excipients. Additional formulation(s) for oral administration and may contain sodium bicarbonate and/or calcium carbonate either as a tablet or liquid, in addition to omeprazole. USP grade, bulk omeprazole was purchased from Esteve Quimica, S.A. (Barcelona, Spain).

At the trial site, the pharmacy staff mixed omeprazole powder with powdered peppermint flavoring and Equal® Sweetener (containing aspartame) [1 part omeprazole: 2 parts peppermint flavoring:1.8 part Equal®]. For each unit dose, 120 mg (containing 40 mg omeprazole powder) was weighed on an analytic balance within 1-2 hours of dose administration in each time period. This mixture was stored under controlled conditions of humidity and temperature.

J. Results

The omeprazole plasma concentrations for 10 subjects of the study are provided below in Table No. 13.

TABLE 13
Omeprazole Concentrations (ng/ml)

| Sub | | Sampling Times (hour) | | | | | | | | | | | | |
|---|---|---|---|---|---|---|---|---|---|---|---|---|---|---|
| No. | Period | 0.00 | 0.08 | 0.17 | 0.25 | 0.50 | 0.75 | 1.00 | 1.50 | 2.00 | 3.00 | 4.00 | 5.00 | 6.00 |
| 1 | 1 | 0.00 | 16.4 | 321 | 738 | 968 | 783 | 605 | 357 | 211 | 97.9 | 40.1 | 16.9 | 11.4 |
| 1 | 2 | 0.00 | 79.3 | 312 | 388 | 441 | 454 | 292 | 200 | 128 | 43.4 | 21.0 | 9.44 | 4.32 |
| 1 | 3 | 0.00 | 0.00 | 0.00 | 0.00 | 0.00 | 0.00 | 0.00 | 39.4 | 120 | 366 | 406 | 161 | 109 |
| 1 | 5 | NS | NS | NS | NS | NS | NS | NS | NS | NS | NS | NS | NS | NS |
| 2 | 1 | 0.00 | 6.82 | 234 | 326 | 582 | 875 | 615 | 322 | 220 | 84.2 | 38.1 | 14.7 | 6.39 |
| 2 | 2 | 0.00 | 47.6 | 84.3 | 168 | 1040 | 717 | 484 | 265 | 162 | 67.6 | 26.2 | 11.6 | 4.02 |
| 2 | 3 | 0.00 | 0.00 | 0.00 | 0.00 | 1.57 | 51.3 | 98.6 | 363 | 379 | 429 | 204 | 99.0 | 51.2 |
| 2 | 5 | 0.00 | 22.9 | 315 | 661 | 983 | 797 | 582 | 375 | 306 | 124 | 57.8 | 25.3 | 12.2 |
| 3 | 1 | 0.00 | 203 | 1230 | 1450 | 1000 | 693 | 525 | 306 | 191 | 79.3 | 32.2 | 14.8 | 7.22 |
| 3 | 2 | 0.00 | 20.6 | 302 | 583 | 831 | 740 | 573 | 336 | 203 | 82.0 | 37.6 | 17.6 | 9.38 |
| 3 | 3 | 0.00 | 0.00 | 0.00 | 0.00 | 9.85 | 57.7 | 179 | 683 | 681 | 345 | 158 | 85.4 | 45.9 |
| 3 | 5 | NS | NS | NS | NS | NS | NS | NS | NS | NS | NS | NS | NS | NS |
| 4 | 1 | 0.00 | 4.57 | 164 | 516 | 1230 | 780 | 495 | 254 | 153 | 55.0 | 20.8 | 8.52 | 3.93 |
| 4 | 2 | 0.00 | 9.53 | 61.6 | 471 | 881 | 566 | 388 | 182 | 107 | 36.5 | 17.9 | 6.17 | 2.63 |
| 4 | 3 | 0.00 | 0.00 | 0.00 | 0.00 | 0.00 | 0.00 | 18.6 | 386 | 454 | 233 | 126 | 81.3 | 51.7 |
| 4 | 5 | 0.00 | 196 | 1240 | 1740 | 994 | 644 | 493 | 305 | 207 | 101 | 44.3 | 18.9 | 8.16 |
| 5 | 1 | 0.00 | 107 | 984 | 1080 | 662 | 409 | 250 | 118 | 60.3 | 19.7 | 7.44 | 2.95 | 1.47 |
| 5 | 2 | 0.00 | 385 | 1400 | 1380 | 693 | 394 | 278 | 144 | 78.1 | 21.8 | 7.20 | 2.16 | BQL |
| 5 | 3 | 0.00 | 0.00 | 0.00 | BQL | 9.25 | 44.0 | 319 | 340 | 252 | 95.5 | 38.8 | 14.6 | 8.16 |
| 5 | 5 | 0.00 | 88.9 | 1210 | 1120 | 677 | 430 | 325 | 173 | 97.8 | 35.1 | 13.4 | 5.04 | 2.06 |
| 6 | 1 | 0.00 | 32.8 | 349 | 552 | 648 | 425 | 267 | 133 | 68.4 | 24.7 | 9.90 | 4.21 | 2.72 |
| 6 | 2 | 0.00 | 13.0 | 68.8 | 101 | 469 | 349 | 241 | 212 | 104 | 31.8 | 9.31 | 3.17 | 1.16 |
| 6 | 3 | 0.00 | 0.00 | 0.00 | 0.00 | 24.0 | 234 | 588 | 351 | 162 | 85.0 | 29.0 | 14.4 | 5.59 |
| 6 | 5 | 0.00 | 5.72 | 26.6 | 50.2 | 190 | 514 | 398 | 177 | 108 | 51.3 | 22.0 | 7.75 | 3.45 |
| 7 | 1 | 0.00 | 5.24 | 97.4 | 269 | 638 | 543 | 431 | 255 | 164 | 63.6 | 29.0 | 11.9 | 5.79 |
| 7 | 2 | 0.00 | 84.0 | 960 | 1170 | 899 | 543 | 433 | 231 | 140 | 54.1 | 24.0 | 12.0 | 5.54 |
| 7 | 3 | 0.00 | 0.00 | 0.00 | 5.42 | 31.0 | 992 | 1110 | 515 | 310 | 115 | 47.0 | 21.8 | 9.32 |
| 7 | 5 | 0.00 | 5.35 | 72.9 | 165 | 363 | 302 | 221 | 268 | 256 | 150 | 71.1 | 29.4 | 11.4 |
| 8 | 1 | 0.00 | 49.9 | 358 | 746 | 1090 | 784 | 609 | 367 | 243 | 104 | 51.1 | 23.1 | 12.1 |
| 8 | 2 | 0.00 | 38.6 | 262 | 1280 | 846 | 563 | 434 | 237 | 148 | 66.9 | 29.5 | 15.7 | 6.15 |
| 8 | 3 | 0.00 | 0.00 | 0.00 | 0.00 | 0.00 | 3.84 | 80.6 | 401 | 313 | 476 | 225 | 108 | 47.1 |
| 8 | 5 | 0.00 | 19.7 | 148 | 582 | 1130 | 822 | 688 | 461 | 264 | 132 | 64.5 | 31.8 | 15.8 |
| 9 | 1 | 0.00 | 16.0 | 139 | 309 | 462 | 355 | 330 | 605 | 317 | 111 | 47.2 | 21.9 | 10.2 |
| 9 | 2 | 0.00 | 277 | 1550 | 1740 | 1150 | 744 | 522 | 305 | 178 | 79.2 | 36.6 | 14.1 | 6.96 |
| 9 | 3 | 0.00 | 0.00 | 0.00 | 0.00 | 0.00 | 1.62 | 47.7 | 551 | 566 | 287 | 153 | 98.0 | 52.5 |
| 9 | 5 | NS | NS | NS | NS | NS | NS | NS | NS | NS | NS | NS | NS | NS |
| 10 | 1 | 0.00 | 15.8 | 130 | 202 | 311 | 233 | 456 | 378 | 187 | 61.6 | 21.2 | 9.90 | 4.20 |
| 10 | 2 | 0.00 | 250 | 1010 | 1100 | 634 | 421 | 310 | 136 | 80.7 | 28.5 | 11.6 | 4.85 | 1.87 |
| 10 | 3 | 0.00 | 0.00 | 0.00 | 0.00 | 5.80 | 114 | 148 | 366 | 390 | 174 | 79.4 | 29.2 | 10.5 |
| 10 | 5 | 0.00 | 103 | 994 | 1190 | 702 | 562 | 353 | 198 | 110 | 36.7 | 14.3 | 5.40 | 2.28 |

NS = No Sample
LOQ = Limit of quantitation: 1.00 ng/ml
BQL = Below quantitation limit

Proton Pump Inhibitor Compositions and Method for Optimizing the Buffer to be Administered in Combination with a Proton Pump Inhibitor

G. Introduction

The compositions of the present invention are designed to produce rapid release of active drug to the site of delivery (typically the stomach) without the necessity of enteric coatings or delayed released dosage forms, while preventing acid degradation of the drug. Acid labile proton pump inhibiting agents, for example, can be formulated or coadministered with one or more buffers sufficient to protect the proton pump inhibitor in any environment, with the ultimate goal being to deliver a proton pump inhibitor to the stomach (or other environment) either via a liquid, a powder or solid dosage form that produces an immediate release of active drug to the site of delivery such that the proton pump inhibitor is quickly available for absorption. Accordingly, Applicant has found that certain amounts of buffers coadministered or mixed with certain proton pump inhibiting agents prevent acid degradation of the proton pump inhibitor when the buffers produce a pH in the stomach or other site of environment that is equal to the pKa of the proton pump inhibitor plus an amount sufficient to protect the proton pump inhibitor from acids and provide undegraded and bioactive proton pump inhibitor to the blood upon administration (e.g., a final pH of pKa of proton pump inhibitor +0.7 log value will reduce the degradation to about 10%). Such buffers should interact with hydrogen ion at rates that exceed the interaction of hydrogen ion with the proton pump inhibitor. Thus, the solubilities of the buffers and proton pump inhibiting agents are important considerations because solubility is a key determinant of the rate of interaction of H+ ion with another compound.

Typically, a proton pump inhibitor formulation of the present invention comprises two primary components: a proton pump inhibitor and an Essential Buffer. An Essential Buffer may include a buffer or combination of buffers that interact with HCl (or other acids in the environment of interest) faster than the proton pump inhibitor interacts with the same acids. When placed in a liquid phase (usually in water), the Essential Buffer produces and maintains a pH of at least the pKa of the proton pump inhibitor. In one embodiment, by raising the pH of the environment to the same of the pKa of the proton pump inhibitor plus about 0.7 log value (or greater), the expected degradation (ionization) can be reduced from about 50% to about 10%. As used herein, the “Essential pH” is the lowest pH of the environment of interest needed to minimize or eliminate the acid-induced degradation of the proton pump inhibitor. The buffering agent(s) employed may raise the pH of the environment to the Essential pH such that 30%, 40% or 50% of the proton pump inhibitor is undegraded, or be present in an amount sufficient to substantially protect (i.e., greater than 50% stability) the proton pump inhibitor.

In another embodiment, the Essential pH is the pKa of the proton pump inhibitor. In a further embodiment, the Essential pH is the sum of the pKa of the proton pump inhibitor plus log 0.7. A log value of about 0.7 is added to the pKa, which represents a decrease of about 5.01187% in stability of the proton pump inhibitor from the pKa plus 1 log value, thus resulting in a stability of approximately 90%, a value widely accepted as desirable in pharmaceutical products. In some cases it may be permissible to accept a value of less than log 0.7.

One aspect of the invention provides that there is also sufficient buffer available to provide the neutralization capacity (Essential Buffer Capacity (“EBC”)) to maintain the elevated pH of the environment (usually gastric) throughout the dwell time that the proton pump inhibitor is passed from the environment and into the blood.

H. Essential Buffers

Essential Buffers can be divided into two groups: Primary Essential Buffers and Secondary Essential Buffers. Every formulation is combined with, either directly or indirectly, at least one Primary Essential Buffer. The Primary Essential Buffers, when used alone or in combination, provide buffering activity below the value that leads to tissue irritation or damage and above a lower limit for the Essential pH of the proton pump inhibitor. Secondary Essential Buffers are not required in every formulation but can be combined with Primary Essential Buffers to produce a higher pH and added neutralization capacity for the formulation.

Determining the type and dose of buffer to protect acid labile substituted benzimidazole proton pump inhibiting agents (and other drugs) is useful for efficacious proton pump inhibitor delivery to and action upon parietal cell proton pumps, particularly when the proton pump inhibitor is administered as an immediate release product designed to disintegrate in the stomach rather than a traditional delayed-release product designed to disintegrate beyond the stomach in higher pH environments such as the duodenum. The present compositions and methods employ determinations of the nature of the buffer(s) to be used, as well as calculations to determine Essential pH, buffering capacity, and volume measurements for individual proton pump inhibitor doses based on their respective solubilities and pKa's. Such inventive methods are applicable for determining the type and amount of buffer(s) necessary to protect the proton pump inhibitor in an array of environments (e.g., mouth, esophagus, stomach, duodenum, jejunum, rectal vault, nasogastric tube, or a powder, tablet, capsule, liquid, etc. in storage before administration). Dosage forms in storage may be exposed to various environments, but a typical set of storage conditions includes storage at room temperature (65-80° F.), and minimal or no exposure to heat, cold, light or humidity as is known in the art.

The present method includes all substituted benzimidazole proton pump inhibiting agents, their salts, esters, amides, enantiomers, racemates, prodrugs, derivatives and the like, and is not limited to those proton pump inhibiting agents used to exemplify the following calculations.

The Essential Buffering Capacity (“EBC”) is the capacity of a proton pump inhibitor/buffer formulation to resist degradation from its environment. The buffering capacity of a proton pump inhibitor/buffer formulation is primarily derived from components of the formulation that possess the ability to combine with acids (H+ ions) from the environment. The EBC contributes to both acid neutralization (antacid effect) and to maintaining an environmental pH>pKa+0.7 to protect proton pump inhibiting agents from acid degradation throughout the dwell time. The Primary Essential Buffer is designed to maintain the pH of stomach contents (or other environment) at a somewhat constant level within a desired range for a period of time so that the proton pump inhibitor can be absorbed from the gastric or other environment. Accordingly, the Essential Buffer is generally more rapid in its complexation with HCl (or other acid) than the proton pump inhibitor administered so that the Essential Buffer is capable of protecting the proton pump inhibitor.

Any weak base, strong base, or combination thereof may be a suitable Essential Buffer. Essential Buffers include, but are not limited to, electrolytes containing the cations sodium, potassium, calcium, magnesium or bismuth. In addition, amino acids, proteins or protein hydrolysates can serve as Essential Buffers owing to their ability to rapidly neutralize acid. When proton pump inhibiting agents are mixed with the Essential Buffer, the proton pump inhibiting agents may be in the free base form, such as omeprazole or lansoprazole; in the sodium salt form, such as esomeprazole sodium, omeprazole sodium, rabeprazole sodium, pantoprazole sodium, etc.; or in a magnesium salt form such as esomeprazole magnesium or omeprazole magnesium or calcium salt forms; or other salt forms. Essential Buffers provide the Essential Buffering Capacity either alone or in combination with Secondary Essential Buffers.

Tribasic sodium phosphate and sodium carbonate are examples of Secondary Essential Buffers for adjusting the pH of any Primary Essential Buffer. Secondary Essential Buffers may assist the Primary Essential Buffer in producing the desirable pH_{E} over the dwell time. Secondary Essential Buffers neutralize HCl (or other acids in the environment) similarly to the Primary Essential Buffers; however, they produce pH values too high to be used alone, as they would lead to gastrointestinal mucosal irritation. They are used to increase the pH and provide additional buffering capacity in combination with a Primary Essential Buffer.

Secondary Essential Buffers do not play an important role in protecting the proton pump inhibitor from early acid-induced degradation. Because they do not work as rapidly, they do not play a major role in proton pump inhibitor protection through the dwell time. Other buffers (“Non-Essential Buffers”) can be added to the Primary and/or Secondary Essential Buffers to provide a latent antacid effect that extends beyond the antacid effect of Essential Buffers.

Many additional buffers can be used, alone or in combination, to achieve an effective buffering capacity for proton pump inhibiting agents or acid labile drugs. A desirable characteristic of buffers includes rapid neutralization of acid environments to greater than pKa+0.7 for the drug being considered.

Non-limiting examples of Primary and Secondary Essential Buffers are set forth in Tables 8 and 9 below.

TABLE 8
Examples of Primary Essential Buffers

| Essential Buffer | Solubility‡ | | pH§ | MW |
|---|---|---|---|---|
| Sodium bicarbonate | 9.96 | g/100 mL | 8-8.4 | 84 |
| Sodium sesquicarbonate | 6.3 | g/100 mL | 9.9-10 | 174 |
| Dibasic sodium phosphate | 10 | g/100 mL | 8.6-9.3 | 142 |
| Sodium tripolyphosphate | 6 | gm/100 mL | 9.7-10 | 368 |
| Tetrasodium pyrophosphate | 5 | g/100 mL | 9.8-10.3 | 266 |
| Sodium citrate | 72 | g/100 mL | 5 | 294 |
| Calcium citrate | 10 | mg/100 mL | 6.8 | 498 |
| Calcium carbonate | 1.5 | mg/100 mL | 6.1-7.1 | 100 |
| Magnesium oxide | 0.62 | mg/100 mL | 9.5-10.5 | 40 |
| Sodium gluconate | 60 | g/100 mL | 6-8 | 218 |
| Sodium lactate | 40 | g/100 mL | 7 | 112 |
| Sodium acetate | 119 | g/100 mL | 8.9 | 82 |
| Dipotassium phosphate | 150 | g/100 mL | 9.3 | 174 |
| Tetrapotassium pyrophosphate | 185 | g/100 mL | 10.4 | 330 |
| Potassium bicarbonate | 36 | g/100 mL | 8.2 | 100 |
| Calcium lactate | 6 | g/100 mL | 7 | 218 |
| Calcium glycerophosphate | 6 | g/100 mL | 7 | 210 |
| Calcium gluconate | 3 | g/100 mL | 7.4 | 430 |
| Magnesium lactate | 10 | g/100 mL | 5.5-7.5 | 269 |
| Magnesium gluconate | 16 | g/100 mL | 7.3 | 414 |

‡solubility is altered by temperature
§pH is altered by concentration and temperature
Note:
hydrated and anhydrous forms are acceptable provided they meet the criteria of a Primary Essential Buffer.

TABLE 9
Examples of Secondary Essential Buffers
These buffers are too caustic to be used alone but
are suitable for addition in low quantities
to the Primary Essential Buffers from Table 8.

| Essential Buffer | Solubility‡ | | pH§ | MW |
|---|---|---|---|---|
| Sodium carbonate | 45.5 | g/100 mL | 10.6-11.4 | 106 |
| Potassium carbonate | | | 11.5 | 138 |
| Sodium phosphate (tribasic) | 8 | g/100 mL | 10.7-12.1 | 163 |
| Calcium hydroxide | 185 | mg/100 mL | 12 | 74 |
| Sodium hydroxide | | | 11.4-13.2 | 40 |

‡solubility is altered by temperature
§pH is altered by concentration and temperature
Note:
hydrated and anhydrous forms are acceptable provided they meet the criteria of a Secondary Essential Buffer.

Amino acids can also be employed as Primary or Secondary Essential Buffers, the doses of which may be calculated according to the following information.

TABLE 10

| | | | | | Solubility |
|---|---|---|---|---|---|
| One | Three | | | | (g/100 g |
| Letter | Letter | | | | H2O at |
| Symbol | Symbol | Amino Acid | MW | pH | 25° C. |
| A | Ala | Alanine | 89 | 6 | 16.65 |
| C | Cys | Cysteine | 121 | 5.02 | Very |
| D | Asp | Aspartic Acid | 133 | 2.77 | 0.778 |
| E | Glu | Glutamic Acid | 147 | 3.22 | 0.864 |
| F | Phe | Phenylalanine | 165 | 5.48 | 2.965 |
| G | Gly | Glycine | 75 | 5.97 | 24.99 |
| H | His | Histidine | 155 | 7.47 | 4.19 |
| I | Ile | Isoleucine | 133 | 5.94 | 4.117 |
| K | Lys | Lysine | 146 | 9.59 | Very |
| L | Leu | Leucine | 131 | 5.98 | 2.426 |
| M | Met | Methionine | 149 | 5.74 | 3.381 |
| N | Asn | Asparagine | 132 | 5.41 | 3.53 |
| P | Pro | Proline | 115 | 6.30 | 162.3 |
| Q | Gln | Glutamine | 146 | 5.65 | 2.5 |
| R | Arg | Arginine | 174 | 11.15 | 15 |
| S | Ser | Serine | 105 | 5.68 | 5.023 |
| T | Thr | Threonine | 119 | 5.64 | Very |
| V | Val | Valine | 117 | 5.96 | 8.85 |
| W | Trp | Tryptophan | 204 | 5.89 | 1.136 |
| Y | Tyr | Tyrosine | 181 | 5.66 | 0.0453 |

References:

IUPAC-IUB Commission on Biochemical Nomenclature (CBN), Rules for Naming Synthetic Modifications of Natural Peptides, (1966); Arch. Biochem. Biophys. 121: 6-8 (1967); Biochem. J. 104: 17-19 (1967), corrected 135: 9 (1973); Biochemistry 6: 362-364 (1967); Biochim. Biophys. Acta 133: 1-5 (1967); Bull. Soc. Chim. Biol. 49: 325-330 (1967) (in French); Eur. J. Biochem. 1: 379-381 (1967), corrected 45: 3 (1974); Hoppe-Seyler's,Z., Physiol. Chem. 348: 262-265 (1967) (in German); J. Biol. Chem. 242 555-557 (1967); Mol. Biol. 2: 466-469 (1968) (in Russian); Pure Appl. Chem. 31: 647-653 (1972); IUPAC Commission on Nomenclature of Organic Chemistry (CNOC), Nomenclature of Organic Chemistry, Stereochem. Rec. E: (1974), Pure Appl. Chem. 45: 11-30 (1976). See also Biochemical Nomenclature and Related Documents, Portland press. 2: 1-18 (1992).

I. The Essential pH (pH_{E})

Substituted benzimidazole proton pump inhibiting agents are labile under acidic conditions. Orally administered proton pump inhibiting agents must be protected from the strongly acidic conditions of the stomach, whether acidic from gastric acids or acids introduced through tube feeds or other sources. In general, the higher the pH of the gastric environment, the greater the stability of the proton pump inhibitor, and thus the more time it has to undergo absorption into the blood and reach and act upon the proton pumps of the gastric parietal cells.

As mentioned, the “Essential pH” is the lowest pH of the environment of interest needed to minimize or eliminate the acid-induced degradation of the proton pump inhibitor during the dwell time in the environment. It is generally expressed herein as pH range. Such pH is the pH of the environment in which the proton pump inhibitor/buffer formulation resides. For example, the environment may be a storage container or the stomach. The environment presents a set of conditions to the proton pump inhibitor/buffer, such as temperature, pH, and the presence or absence of water. The dwell time is the time that the proton pump inhibitor dwells in a specific environment, i.e., the GI tract prior to its passage into a different environment, i.e. the blood serum. The shelf-life is another example of a dwell time, in which case, the specific environment may be a container of dry, powdered formulation. As used herein, “Resultant pH” is the pH that is the result of adding a proton pump inhibitor/buffer formulation to an environment of interest. “Formulation pH” is the pH of the proton pump inhibitor/buffer formulation when it is in liquid form.

A proton pump inhibitor dose within its calculated pH_{E} range is designed to ensure sufficient proton pump inhibitor protection from acid degradation such that delivery to and action upon proton pumps occur. In one desirable embodiment, the pH_{E} is the sum of the pKa of a given proton pump inhibitor plus about 0.7. The pKa is defined as the pH at which 50% of a chemical is in the ionized form. When the pH of the environment equals the pKa of the proton pump inhibitor, then 50% ionization (degradation) of the proton pump inhibitor occurs. However, by adding the factor of 0.7, this ionization is reduced to 90%.

The Stability Range Factor (“SRF”) is the range of pH elevation in which the lower limit is the sum of the pKa of a given proton pump inhibitor +0.7 log, and the upper limit is the pH at which elimination of acid degradation occurs without producing tissue irritation from extreme alkalinity. SRF is calculated based on the desirable shelf-life (or a dwell time), the environmental pH and the amount of acid expected to be encountered, along with a knowledge of the time of exposure expected after the drug is administered and before the drug reaches the blood (i.e., the dwell time).

The upper limit of the SRF is a function of the tolerability of the gastrointestinal mucosa to alkaline substances, which is determined by the Formulation pH and the concentration of alkaline material presented. For practical purposes, pH=10.9 delineates an upper limit of the SRF. It is acknowledged that the amount of buffer is an important aspect of the tissue destructive potential of an alkaline substance. Therefore, the SRF for any given proton pump inhibitor begins at the sum of the pKa of the proton pump inhibitor +0.7, and extends upwards to a pH of about 10.9.

The Essential pH used with the SRF establishes a desirable range for the stability to the actions of H+ ion (or other acidic component) on the proton pump inhibitor/buffer formulation. Sufficient buffering capacity maintains an Essential pH as described below as “Essential Buffering Capacity.”

Examples of pH_{E} calculations with SRF for specific proton pump inhibiting agents are as follows:
pH_{E} of proton pump inhibitor=pKa of proton pump inhibitor +0.7.
SRF=the range: pH_{E} to 10.9.
SRF for omeprazole=(pKa omeprazole+0.7) to 10.9=(3.9±0.7)=4.6 to 10.9.
SRF for lansoprazole=(pKa lansoprazole+0.7) to 10.9=(4.1±0.7)=4.8 to 10.9.
SRF for rabeprazole=(pKa rabeprazole+0.7) to 10.9=(4.9±0.7)=5.6 to 10.9.
SRF for pantoprazole=(pKa pantoprazole+0.7) to 10.9=(3±0.7)=3.7 to 10.9.

In most instances, the lower end of each of the above ranges is increased by one pH unit to minimize, by a factor of 10, any local effects within the stomach that may produce areas of lower pH that might cause proton pump inhibitor degradation. A value of +1 log value is also supported by the observation that weak bases operate most efficiently at neutralizing acid beginning at +1 log value above the pKa.

For example, one would expect to encounter about 100-150 ml of 0.11 to 0.16N HCl in the adult fasting stomach, which is equivalent to about 12-24 mEq of HCl. Therefore, an equal amount of base will neutralize this acid. If about 12-24 mEq of sodium bicarbonate is employed as the buffer, the resulting pH will be left at the pKa of the conjugate acid of sodium bicarbonate (carbonic acid), which is about 6.14 or greater. This is greater than the lower limit of the pH_{E} for omeprazole of 4.6. Thus, administering 12-24 mEq of sodium bicarbonate with omeprazole protects greater than 95% of the drug when encountering 12-24 mEq of HCl. Because sodium bicarbonate complexes with HCl at a rate that exceeds the rate of interaction of omeprazole, it is considered a suitable buffer.

It should be noted that depending on age and disease, the amount of acid to be encountered can be significantly more or less than the 12-24 mEq range, but is generally from about 4 mEq to about 30 mEq.

Using magnesium oxide or magnesium hydroxide in an amount of 12 to 24 mEq also provides sufficient neutralizing capacity leaving the pH at approximately 7 (lowered only slightly by the minimal hydrolysis of magnesium). However, magnesium hydroxide is not rapid in onset and care should be taken to ensure that early degradation of the proton pump inhibitor does not occur. Early degradation can be avoided by making a tablet comprising two layers: an inner layer of proton pump inhibitor and sodium bicarbonate, and an outer layer of magnesium hydroxide dried gel or magnesium oxide with suitable disintegrant such that the magnesium oxide would rapidly disintegrate in the stomach. Alternatively, the inner layer can contain the magnesium buffer and the outer layer has the proton pump inhibitor and sodium bicarbonate.

Additionally, micronization of the slower acting buffer can be used to enhance its ability to combine with acid. Calcium carbonate (and many other calcium buffers) is a similar slower acting (compared to sodium bicarbonate) but potent buffer. Therefore, if used, it would be best suited in an outer layer of a tablet formulation with the inner layer comprising a rapid acting buffer with proton pump inhibitor (or vice versa). Alternatively, mixtures of the buffers can be employed for the outer layer. If developing a liquid formulation or a powder for reconstitution, a mixture of a rapid acting buffer and slower acting buffer can be used (e.g., sodium bicarbonate and magnesium oxide, respectively).

Modifications to the formulations may entail adjusting the pH of products with basic or acidic chemicals, including but not limited to, chemicals described throughout this application. Modifications of buffer pH based on the pH_{E} may or may not be performed in specific instances, depending upon species, age, disease and other variations between patients.

J. pKa and Solubility of Proton Pump Inhibiting Agents

As mentioned above, the pKa of a given proton pump inhibitor indicates inherent stability with respect to acid degradation; the lower the pKa, the more stable the proton pump inhibitor. The solubility of the proton pump inhibitor will also dictate the rate at which the proton pump inhibitor complexes with, and is degraded by, acid. These two physicochemical characteristics (pKa and solubility) of the proton pump inhibitor interact with the physicochemical characteristics of the buffer(s) (pH, buffering capacity and rate of buffering action) in the presence of acid in the environment to determine the degradation of the proton pump inhibitor over time. The less soluble a proton pump inhibitor is in water, the lower the initial degradation when placed in an acidic environment. The following Table 11 elaborates on the time for 50% of drug to be degraded (t ½), pKa and solubility in water of several proton pump inhibiting agents.

TABLE 11

| | Pantoprazole | | | | | | Rabeprazole | |
|---|---|---|---|---|---|---|---|---|
| PH | sodium | | Omeprazole | | Lansoprazole | | sodium | |
| 1.2 | 4.6 | min | 2.8 | min | 2.0 | min | 1.3 | min |
| 5 | 2.8 | hr | 1.0 | hr | 1.1 | hr | | |
| 5.1 | 4.7 | hr | 1.4 | hr | 1.5 | hr | 7.2 | minutes |
| 6 | 21 | hr | 7.3 | hr | 6.4 | hr | | |
| 7 | 73 | hr | 39 | hr | 35 | hr | | |
| PKa | 3 | | 3.9 | | 4.1 | | 4.9 | |

| | | | | |
|---|---|---|---|---|
| Solubility | very soluble | slightly | very slightly | Very soluble |
| | | soluble | soluble | |

Kromer W, et al. Differences in pH-Dependent Activation Rates of Substituted Benzimidazoles and Biological in vitro Correlates, PHARMACOLOGY 1998; 56: 57-70.

Although pantoprazole sodium, with a pKa of 3, is inherently more stable in an acidic environment than other proton pump inhibiting agents, it is also very soluble in water and thus could undergo 50% degradation in an acidic stomach with a pH of 1.2 in less than 5 minutes. Therefore, it is important for the buffer(s) used with pantoprazole sodium to interact with H+ ion (or other acidic substances) more rapidly than the pantoprazole sodium interacts with such acids and maintain the rapid complexation through the dwell time; otherwise, additional dosing of buffer may be required. The overall pH of the gastric contents should be kept at least at the pKa+0.7 (i.e., 3.7) from the time the proton pump inhibitor in solution comes into contact with the gastric acid continuing throughout the dwell time. Essential Buffers for liquid formulations of pantoprazole sodium include those buffers whose conjugate acids possess a pKa>3.7 and which are very soluble (e.g., potassium bicarbonate and sodium bicarbonate) Oral solid formulations likewise would require buffers whose conjugate acid possesses a pKa>3.7 and rapid complexation potential. Most magnesium, calcium and aluminum salts are not suitable unless the pantoprazole sodium is placed (with or without additional buffer) in an inner portion of a tablet or capsule with such antacids, and surrounded by a rapid acting buffer with a rapid disintegrant. Another formulation method for pantoprazole is to decrease its solubility such as by selecting a less soluble salt form or the non-salt form, pantoprazole.

Rabeprazole sodium is also very soluble in water and could undergo 50% degradation in an acidic stomach with a pH of 1.2 in less than 1.5 minutes. It is not very stable to acid degradation due to its higher pKa of 4.9. A suitable buffer(s) for rabeprazole sodium interacts with H+ ion (or other acidic substances) more rapidly than the rabeprazole sodium interacts with such acids to prevent early degradation, and should possess high neutralizing capacity to enable rabeprazole to survive through the dwell time. Sodium or potassium bicarbonate would be good choices in this instance.

Another option for rabeprazole sodium (as well as any sodium salt of a proton pump inhibitor, which would tend to be more soluble than the base form) is to reduce the solubility of rabeprazole sodium when in aqueous form such as using a less soluble salt form or using the non-salt form. This decreases early degradation because the rabeprazole must first undergo dissolution in water before it is degraded by acid. In this embodiment, the suitable buffer(s) for rabeprazole sodium should possess high neutralizing capacity to enable rabeprazole to survive through the dwell time.

For proton pump inhibiting agents that possess high pKa's, such as rabeprazole sodium, a two-part liquid formulation can be utilized. The liquid part has the proton pump inhibitor and a high pH, but a low mEq buffering capacity. The liquid part is added to a second part that possesses a lower pH but a higher mEq buffering capacity. When these two parts are added together just prior to administration, a formulation with a lower pH and a higher buffering capacity is produced which will neutralize stomach acid but not be too caustic to tissues. Examples of such formulations are provided below.

For highly soluble proton pump inhibiting agents, the formulation may be produced in a solid dosage form such as a tablet, capsule or powder with a buffer(s), which disintegrate and reach solution at a rate that exceeds the proton pump inhibitor and thereby provides the Essential pH for protection of the proton pump inhibitor prior to its dissolution and interaction with the acid in the environment. Further, the tablet or capsule may be formulated to possess an outer portion of buffer and an inner portion comprising proton pump inhibitor, or a blend of proton pump inhibitor and buffer. Additional methods include formulating the buffer in a smaller particle size (e.g., micronized) and the proton pump inhibitor in a larger particle size. This results in the disintegration of the buffer component prior to disintegration of the proton pump inhibitor component. All of these methods of formulation aim to create an environment of stability for the proton pump inhibitor during the dwell time.

The dosage form may affect the suitability of a buffer for use in a formulation. For example, magnesium oxide is a buffer with high buffering capacity but slow onset when formulated as a tablet. However, when formulated as a powder, or a tablet of low compression, or with tablet disintegrants such as pregelatinized starch, it disintegrates more rapidly.

Omeprazole base is only slightly soluble in water and, as such, less of the drug is subject to early and continued degradation. The soluble portion of omeprazole is vulnerable to early degradation in the gastric environment. Dissolution of the remaining insoluble portion is expected to occur within minutes of encountering the water of the gastric secretions. This dissolution time provides some protection against early degradation provided that relatively low volumes of water are used during delivery or in the product formulation. After several minutes in the gastric environment, upon complete dissolution, omeprazole could undergo 50% degradation in less than 3 minutes. Omeprazole is moderately stable owing to its pKa of 3.9. A suitable buffer(s) for omeprazole is rapid acting and possesses at least moderate neutralizing capacity to enable omeprazole to survive through the dwell time.

As used herein, “rapid acting” in the context of a buffer means a buffer that raises the pH of the environment to greater than or equal to the pH_{E} of a particular proton pump inhibitor in a time sufficient to prevent significant degradation of the proton pump inhibitor. In one embodiment, the rapid acting buffer raises the pH to at least the pKa of the proton pump inhibitor plus 0.7 log value within 10 minutes.

Preferred buffer(s) produce an environment where the Resultant pH of the environment is equal to or greater than the Essential pH such that: (1) the onset of pH change to equal to or greater than the pH_{E}+0.7 begins before the acid-induced degradation of the proton pump inhibitor occurs, and (2) the Resultant pH at or greater than the pH_{E}+0.7 lasts throughout the dwell time, which is typically a minimum of 30 minutes in the case of gastric emptying for an adult. It is desirable that the buffer be rapid acting to minimize early acid-induced degradation. The most rapid acting buffers are water soluble (or soluble in the environment). High solubility, however, is not an absolute necessity as magnesium oxide and calcium carbonate, both only slightly soluble, are capable of significant complexation with gastric acid albeit at a slower rate. If a dry formulation is used, such as a tablet, the particle size of the buffer(s) can be reduced to enhance the dissolution rate while the particle size of the proton pump inhibitor can be increased. Disintegrants can be added to enhance the availability of poorly soluble buffers.

Lansoprazole base is very slightly soluble in water and, as such, less of the drug is subject to early degradation. The soluble portion is vulnerable to early degradation. Dissolution of the remaining insoluble portion is expected to occur within several minutes of encountering the water of the gastric secretions. This dissolution time provides some protection against early degradation provided that relatively low volumes of water are used for delivery or in the product formulation. After several minutes, upon complete dissolution, lansoprazole could undergo 50% degradation in 2 minutes. Lansoprazole is moderately stable owing to its pKa of 4.1. A suitable buffer(s) for lansoprazole should be rapid acting, and should possess moderate to high neutralizing capacity to enable lansoprazole to survive through the dwell time. The pH of the gastric contents (or other environment) should be kept at greater than about 4.8 from the time the proton pump inhibitor in solution comes into contact with the gastric acid continuing throughout the dwell time.

K. Calculating the Acid Neutralizing Capacity of Buffers

The acid neutralizing capacity (“ANC”) of soluble buffers may be used to assist in selecting a preferred amount of buffer(s) needed to provide the EBC. The ANC uses both the formula weight (FWt.) and the valence to determine buffering capacity.

An example of an ANC calculation for sodium bicarbonate is as follows:
Sodium Bicarbonate, Na⁺HCO₃⁻, FWt.=84, valence=1. The conversion equation from equivalent weight to grams is:
(Equivalent Weight (“EW”))(1/1000 mmol)(1 mmol/1 mEq)=grams of NaCHO₃
EW=(FWt.)/(valence)=84/1=84 g/mol.
(84 g/mol)(1 mol/1000 mmol)(1 mmol/1 mEq)(4 mEq)=0.34 g NaHCO₃ needed for 4 mEq of buffering capacity.

Accordingly, for 10 mEq, one needs 0.840 g NaHCO₃, and for 30 mEq, 2.52 gm is required. The range of 4-30 m Eq is used because that is the range of mEq of acid to be encountered in most patients.

The ANCs of other buffers are similarly calculated. ANC determinations are from Drake and Hollander, Neutralizing Capacity And Cost Effectiveness Of Antacids, ANN INTERN. MED. 109:215-17 (1981). Generally, the formulations of the present invention need about 4 to about 30 mEq of buffering capacity although higher amounts could be used in some patients.

Sodium bicarbonate in solution possesses a pH>pH_{E} of omeprazole and rapidly neutralizes acidic environments. As stated above, rapid complexation with HCl is a desirable characteristic of an Essential Buffer. Ideally, but not necessarily required as indicated in formulations that contain a tablet in a tablet, the Essential Buffer complexes with the acid at a faster rate than the proton pump inhibitor it is intended to protect.

In selecting Essential Buffers, a knowledge of buffering capacity is also useful since they possess differing pHs at various concentrations. The magnitude of the resistance of a buffer to pH changes is referred to as buffer capacity (Beta). It has been defined by Koppel, Spiro and Van Slyke as the ratio of the increment of strong acid (or base) to the change in pH brought about by addition of acid. The following formula is used to measure buffer capacity: Buffer capacity=the increment (in gram equivalents per liter) of strong acid added to the buffer solution to produce a pH change (change as measured in absolute terms), or buffer capacity=change in acid/change in pH. Improvements in the formula have been made to improve the precision, and these form the basis for mathematical comparison of buffers for consideration. See Koppel, BioChem, Z. (65) 409-439 (1914), Van Slyke, J. BIOL. CHEM. 52:525 (1922).

When the proton pump inhibitor/buffer formulation is placed in the environment, the proton pump inhibitor is subject to degradation by the acid in that environment. As depicted in FIG. 9, proton pump inhibitor solubility, the pKa of the proton pump inhibitor, and the amount and concentration of acid (H+ ion) encountered in the environment are variables that can be used to determine the appropriate candidate as an Essential Buffer. Early degradation occurs when the soluble portion of the proton pump inhibitor (that portion available for immediate interaction with H+ ion) undergoes hydrolysis by H+ ion. proton pump inhibiting agents differ in their solubility and, therefore, those that are more soluble have a potential for a higher portion of proton pump inhibitor degraded by early interaction with H+ ion. The pKa of the proton pump inhibitor and the pH of the environment of the stomach (or other site of interest) after addition of the proton pump inhibitor/buffer formulation (Resultant pH) can be used to determine the desirable Essential Buffer. By measuring the Resultant pH over time, the pH data versus time can be plotted as seen in FIG. 9. The graph of pH over time can then be used to evaluate various buffers.

Such a graph can be developed for a potential buffer or buffer combination using the Rossett-Rice test (Rosset N E, Marion L: An In Vitro Evaluation Of The Efficacy Of The More Frequently Used Antacids With Particular Attention To Tablets. ANTACIDS 26: 490-95 (1954), modified with continual addition of simulated gastric fluid. See USP XXIII, The United States Pharmacopoeia, 23^{rd} Revision, United States Pharmacopeia Convention, Inc. Briefly, the test employs 150 mL of simulated gastric fluid consisting of 2 Gm of sodium chloride and 3.2 Gm of pepsin, which are dissolved in 7 mL of 1N HCl, q.s. to 1000 mL with distilled water. The pH of the simulated gastric fluid is 1.2. A container of 150 mL of this fluid is stirred at 300 rpm±30 rpm with a magnetic stirrer and kept at 37.1° C. A pH electrode is kept in the upper region of the solution. The test buffer or the subject formulation is added to the container to start the evaluation. At 10 minutes, a continuous drip of simulated gastric fluid is added to the test container at a rate of 1.6 ml/min to simulate gastric secretion. Approximately 1.6 mL/min is removed from the test container to keep the volume in the test container constant. The evaluation continues for at least 90 minutes.

This methodology allows for a dynamic evaluation of buffering capacity in a model designed to mimic a fasting human stomach. It has been described in part for use in evaluating antacids by Beneyto J E, et. al., Evaluation of a New Antacid, Almagate, ARZNEIM-FORSCH/DRUG Res 1984; 34 (10A):1350-4; Kerkhof N J, et al, pH-Stat Tiration of Aluminum Hydroxide Gel, J. PHARM. SCI. 1977; 66: 1528-32.

Using this method, a pH tracing can be developed for evaluating buffers as well as finished products. In addition, a sample of the test solution can be taken during the experiment to evaluate the extent of proton pump inhibitor degradation at various times. Those buffers with a suitable profile as exemplified in FIG. 9 able to maintain pH greater than or equal to pH_{E} for 30 minutes or greater, can be considered suitable Essential Buffers. In one embodiment, as depicted in FIG. 9, the pH was recorded over 10 second intervals.

A number of buffers may be applicable for use as Essential Buffers. Therefore, once an Essential Buffer is chosen, the amount necessary to provide the EBC is calculated. As used herein, the EBC is the buffering capacity, or amount of alkaline buffer, included in the dose and calculated to maintain the Essential pH range and thereby protect any substituted benzimidazole proton pump inhibitor in the gastric (or other) environment. In patients requiring continuing proton pump inhibitor administration (e.g. daily), more buffering capacity may be necessary with the first dose or first few doses than with subsequent doses because the proton pump inhibitor may encounter more acid with the initial doses. Subsequent doses will require less buffering capacity because the initial proton pump inhibitor doses will have reduced gastric acid production. The EBC could therefore be reduced in subsequent doses. The product's buffering capacity may be formulated as desired, for instance with respect to patient age, gender or species.

Experimental data from adult human subjects showed an effective EBC range of a first dose of omeprazole to be about 4 to about 20 mEq (“EBC-O range”) of sodium bicarbonate, with a range of about 12 to about 25 mEq suitable in most instances. Subsequent doses of omeprazole require less EBC, with a range of about 4 to 15 mEq sodium bicarbonate. In one embodiment, this latter EBC range proved optimal for an omeprazole suspension administered to patients with varying degrees of gastrointestinal transit and acid output, based on a knowledge of basal and maximal acid outputs of 2 and 25 mEq/hour, respectively. These studies have been reported in Phillips J. O. et al., CRIT. CARE MED. 1996; Lasky et al., J. TRAUMA 1998.

Based on the EBC-O range, the above ANC calculation can be employed. Additionally, it is expected to encounter about 100-150 mL of 0.1 N HCl (equating to about 12-24 ieq of acid) in a fasting stomach. Variations in the acid encountered in the environment will affect the Essential Buffering Capacity required. The above EBC ranges relate to adult patients. Children, however, produce less acid per unit time in comparison to adults. Therefore, depending on the patient population, the amount of Essential Buffering Capacity required may be altered.

Numerous references are available to assist the skilled artisan in identifying a suitable buffer companion with a proton pump inhibitor to determine the desirable characteristics stated herein. See, e.g., Holbert, et. al., A Study of Antacid Buffers: I. The Time Factor in Neutralization of Gastric Acidity, J. AMER. PHARM. ASSN. 36: 149-51 (1947); Lin, et. al., Evaluation of Buffering Capacity and Acid Neutralizing pH Time Profile of Antiacids, J. FORMOSA MED. ASSN. 97 (10) 704-710 (1998); Physical Pharmacy, pp 169-189; Remington: The Science and Practice of Pharmacy (2000).

L. The Desirable Volume

The Desirable Volume (“DV”) of a proton pump inhibitor dose may affect proton pump inhibitor delivery to and action upon parietal cell proton pumps. The DV of a dose is partly based on the EBC. For liquid formulations, a desirable volume should deliver sufficient buffer to act as an antacid to neutralize a substantial amount of gastric or other acids. For solid formulations such as tablets, a nominal amount of water or other fluid will be consumed to aid in swallowing the tablet. Liquid preparations of the present invention use volumes as small as about 2 ml or in excess of about 60 ml. Volumes smaller than 2 ml and larger than 60 ml are contemplated, and may be used as desired to suit individual patients, such as those of advanced or very young age or of different species. Very large volumes may lead to higher amounts of less soluble proton pump inhibiting agents (e.g., omeprazole, lansoprazole base forms) going into solution, which could result in vulnerability to early degradation.

For instance, volumes smaller than about 2 ml may be used in newborns or premature infants, or in small animals, because of their smaller stomach size. Also, a large DV may be required for doses formulated with dilute buffer concentrations, to achieve the EBC. The relationship between the EBC and DV is in part shown below:
If EBC(mg buffer)=Buffer conc.(mg/ml)×DV(ml),
then DV(ml)=EBC(mg)/Buffer conc.(mg/ml).

Alternatively, mEq can be substituted for mg in the formula.

M. Secondary Components of the Formulations

Secondary components are not required but may be used to enhance the pharmacological action or as pharmaceutical aids. Secondary components may include, but are not limited to, parietal cell activators and other ingredients. Parietal cell activators, as discussed above, are compounds that produce an increase in proton pump activity such that proton pumps are relocated from storage sites of the parietal cell, i.e. tubulovesicles, to the site of H+, K+ exchange at the secretory canaliculus. A parietal cell activator may also serve other functions. For example, sodium bicarbonate is an Essential Buffer as well as a parietal cell activator, chocolate is a parietal cell activator and a flavoring agent, and aspartame, which contains phenylalanine, is a sweetener as well as a parietal cell activator.

Parietal cell activators can be divided into four groups: 1) rapid acting buffers that are weak bases, strong bases or combinations thereof that also produce a rapid onset of effect (the pH drops rather suddenly after the buffer is exhausted; these buffers typically cause the pH of the stomach to rise to above 5); 2) amino acids, protein hydrolysates and proteins; 3) calcium containing compounds such as calcium chloride or calcium carbonate; and 4) compositions such as coffee, cocoa, caffeine and peppermint.

The other ingredients comprise components of a formulation that are secondary to the primary components. Other ingredients include, but are not limited to, thickening agents, flavoring agents, sweeteners, antifoaming agents (such as simethicone), preservatives, antibacterial or antimicrobials agents (such as cefazolin, amoxicillin, sulfamethoxazole, sulfisoxazole, erythromycin and other macrolides such as clarithromycin or azithromycin), and Secondary Essential Buffers.

Desirable flavoring agents may be added to the dosage forms, and may or may not need to be buffered to the pH_{E}. Flavoring agents with pH values inherently suitable to the range of pH_{E} values of proton pump inhibiting agents include, but are not limited to, apple, caramel, meat, chocolate, root beer, maple, cherry, coffee, mint, licorice, nut, butter, butterscotch, and peanut butter flavorings, used alone or in any combination. Similarly, all substances included in the formulation of any proton pump inhibitor product, including but not limited to, activators, antifoaming agents, potentiators, antioxidants, antimicrobial agents, chelators, sweeteners, thickeners, preservatives, or other additives or substances may be buffered to the pH_{E}.

N. Examples Utilizing the Calculations

The pH_{E}, the EBC, and the DV of a proton pump inhibitor dose may affect proton pump inhibitor delivery to, and action upon, parietal cell proton pumps. The following calculations tailor an Essential Buffer dose for any substituted benzimidazole proton pump inhibitor to promote proton pump inhibitor efficacy in an oral administration.

Calculation 1: To deliver a 20 mg dose of omeprazole (pKa=3.9) in sodium bicarbonate:
Step 1: The pH_{E} of omeprazole=pKa of omeprazole+0.7=4.6. The SRF of omeprazole=pH_{E} to 10.9=4.6 to 10.9. At a Formulation pH of 4.6 to 10.9, the conjugate base of sodium bicarbonate (carbonic acid) has a pKa of 6.14. Therefore, an amount of sodium bicarbonate equivalent to the amount of acid to be encountered would produce a pH of 6.14, which is within the SRF of 4.6 to 10.9. Sodium bicarbonate would make a suitable choice as a buffer.
Step 2: The EBC=4 to 30 mEq buffering capacity equivalent.
Step 3: To determine the amount of sodium bicarbonate to administer with the omeprazole, the ANC for sodium bicarbonate is calculated. The ANC for sodium bicarbonate (MW=84 for 4-30 mEq)=(EW)(1/1000 mmol)(1mmol/1 mEq)(EBC)
   EW=MW/(valence)=84/1=84 g/mol
(84 g/mol)(1 mol/1000 mmol)(1 mmol/1 mEq)(4 to 30 mEq)=0.34 g to 2.52 g
Step 4: For liquid formulations, if the DV=20 ml, then DV=Essential Buffer (EB)(mg)/Buffer conc. (mg/ml)
Buffer conc.=EB/DV=340 mg to 2520 mg/20 ml=17 mg/ml to 126 mg/ml.

Therefore, for 20 mg of omeprazole to be adequately buffered in 20 ml of solution, the concentration of sodium bicarbonate should be 17 to 126 mg/ml.

Calculation 2: To deliver a 20 mg dose of omeprazole (pKa=3.9) in dibasic sodium phosphate:
Step 1: The pH_{E} of omeprazole=pKa of omeprazole+0.7. The SRF of omeprazole=(3.9±0.7) to 10.9=4.6 to 10.9.
Step 2: The EBC=4 to 30 mEq buffering capacity equivalent.
Step 3: To determine the amount of dibasic sodium phosphate to administer with the omeprazole, the ANC for dibasic sodium phosphate is calculated. The ANC for dibasic sodium phosphate (MW=142)=(EW)(1/1000 mmol)(1 mmol/1 mEq)(EBC).
   EW=MW/(valence)=142/2=71 g/mol.
(71 g/mol)(1 mol/1000 mmol)(1 mmol/1 mEq)(4 to 30 mEq)=0.28 g to 2.13 g
Step 4: For liquid formulations, if the DV=20 ml, then DV=EB (mg)/Buffer conc. (mg/ml)
Buffer conc.=EB/DV=280 mg to 2130 mg/20 ml=14 mg/ml to 107 mg/ml.

Therefore, for 20 mg of omeprazole to be adequately buffered in 20 ml of solution, the concentration of dibasic sodium phosphate should be 14 to 107 mg/ml. The pka of disodium phosphate is 7.21. Therefore, an amount of disodium phosphate equivalent to the amount of acid to be encountered would produce a pH of approximately 7.2. Thus, disodium phosphate would make a suitable choice as a buffer. Calculation 3: To deliver a 30 mg dose of lansoprazole (pKa=4.1) in sodium bicarbonate:
Step 1: The pH_{E} of lansoprazole=pKa of lansoprazole+0.7. The SRF of lansoprazole=(4.1±0.7) to 10.9=4.8 to 10.9.
Step 2: The EBC=4-30 mEq buffering capacity equivalent.
Step 3: To determine the amount of sodium bicarbonate to administer with the lansoprazole, the ANC for sodium bicarbonate is calculated. The ANC for sodium bicarbonate (MW=84)=(EW)(1/1000 mmol)(1 mmol/1 mEq)(EBC)
   EW=MW/valence=84/1 g/mol
(84 g/mol)(1 mol/1000 mmol)(1 mmol/1 mEq)(4 to 30 mEq)=0.34 g to 2.52 g
Step 4: For liquid formulations, if the DV=20 ml, then DV=EB (mg)/Buffer conc. (mg/ml)
Buffer conc.=EB/DV=340 mg to 2520 mg/20 ml=17 mg/ml to 126 mg/ml.

Therefore, for 30 mg of lansoprazole to be adequately buffered in 20 ml of solution, the concentration of sodium bicarbonate should be about 17 to about 126 mg/ml.

Calulation 4: To deliver a 40 mg dose of pantoprazole (pKa=3) in sodium bicarbonate:
Step 1: The pH_{E} of pantoprazole=pKa of pantoprazole+0.7. The SRF of pantoprazole=(3+0.7) to 10.9=3.7 to 10.9.
Step 2: The EBC=4-30 mEq buffering capacity equivalent.
Step 3: To determine the amount of sodium bicarbonate to administer with the pantoprazole, the ANC for sodium bicarbonate is calculated. The ANC for sodium bicarbonate (MW=84)=(EW)(1/1000 mmol)(1mmol/1 mEq)(EBC)
   EW=MW/(valence)=84/1 g/mol
(84 g/mol)(1 mol/1000 mmol)(1 mmol/1 mEq)(4 to 30 mEq)=0.34 g to 2.52 g
Step 4: For liquid formulations, if the DV=20 ml, then DV=EB (mg)/Buffer conc. (mg/ml)
Buffer conc.=EB/DV=340 mg to 2520 mg/20 ml=17 mg/ml to 126 mg/ml.

Therefore, for 40 mg of pantoprazole to be adequately buffered in 20 ml, the concentration of sodium bicarbonate should be about 17 to 126 mg/ml.

Calculation 5: To deliver a 20 mg dose of rabeprazole (pKa=5) in sodium phosphate dibasic:
Step 1: The pH_{E} of rabeprazole=pKa of rabeprazole+0.7. The SRF of rabeprazole=4.9±0.7) to 10.9=5.6 to 10.9.
Step 2: The EBC=4-30 mEq buffering capacity equivalent.
Step 3: Therefore, to determine the amount of sodium phosphate dibasic to administer with the rabeprazole, the ANC for potassium sodium dibasic is calculated. The ANC for sodium phosphate dibasic (duohydrate) (MW=174)=(EW)(1/1000 mmol)(1 mmol/1 mEq)(EBC)
   EW=MW/valence=178/1 g/mol
(178 g/mol)(1 mol/1000 mmol)(1 mmol/1 mEq)(4 to 20 mEq)=0.712 g to 5.34 g sodium phosphate dibasic.
Step 4: For liquid formulations, if the DV=20 ml, then DV=EB (mg)/Buffer conc. (mg/ml).

Buffer conc.=EB/DV=0.712 g to 2 g/20 ml=35.6 mg/ml to 100 mg/ml. In this case, the solubility of disodium phosphate would limit the amount that could be dissolved in 20 mL. Obviously, this would exceed the solubility of disodium phosphate (sodium phosphate dibasic). Therefore, for 20 mg of rabeprazole to be adequately buffered in 20 ml of solution, the concentration of sodium phosphate dibasic should be about 35.6 mg/ml 10 to 100 mg/ml at a pH range of about 6.9 to 10.9. The pka of disodium phosphate is 7.21. Thus, an amount of disodium phosphate equivalent to the amount of acid to be encountered would produce a pH of approximately 7.2. Accordingly, disodium phosphate would make a suitable choice as a buffer.

It should be noted that the suitability of buffers relates to their use immediately after mixing. In order to enhance the shelf-life, higher pH values would be anticipated within the range of acceptable pH_{E} for a given proton pump inhibitor. As an example, rabeprazole suspensions containing various buffers were evaluated for color change because degradation of proton pump inhibiting agents results in a color change to brown or black. All buffer suspensions started out white in color. After 2 weeks the following observations were made:

20 mg Rabeprazole in Various Buffers Stored
Under Refrigerated Conditions As Suspensions

| | Original | Color | pH at |
|---|---|---|---|
| Buffer | Color | 14 days | 14 days |
| Sodium bicarbonate | white | brown | 8.3 |
| 800 mg/10 mL | | | |
| Disodium phosphate | white | white | 10.3 |
| 800 mg/10 mL | | | |
| Disodium phosphate | white | white | 10.5 |
| 700 mg; | | | |
| Trisodium phosphate | | | |
| 100 mg/10 mL | | | |

Similar calculations may be performed for any substituted benzimidazole proton pump inhibitor and appropriate buffer(s) including, but not limited to, those exemplified above. One skilled in the art will appreciate that the order of the above steps is not critical to the invention. The above calculations may be used for formulations comprising one or more proton pump inhibitor and one or more buffers.

O. Veterinary Formulations

Horses produce stomach acid continuously throughout the day. It is the basal acid secretion from the stomach in the absence of feeding that is responsible for the erosion of the squamous mucosa in the stomach and ulcers. Horses on pasture normally secrete a continuous supply of saliva, which buffers the stomach acid. When horses are being ridden regularly, trained for shows or prepared for sales, they are usually kept in stalls much of the day. Under these conditions, the natural salivary buffering mechanism is disrupted and acid indigestion often results.

Almost 40 to about 100 mEq of buffer capacity should provide approximately 2.5 hours of neutralization for a horse. The usual dose of omeprazole ranges from 0.7 to 1.5 mg/kg/day (doses up to 4 mg/kg/day may be required) and a typical weight for a horse is 500 kg. Similar dosages are expected for rabeprazole and lansoprazole.

Dogs can also suffer from ulcers and their dosage is approximately 1 mg/kg/day. The following formulations are designed for use in horses but smaller amounts can be used in dogs with an EBC of 10 to 20 mEq.

Formulation 5: Veterinary Formulation of Omeprazole

This formulation is particularly well suited for animals rather than
humans because the dose of proton pump inhibitor is high.
EBC = 75 mEq
Essential pH (omeprazole pKa = 3.9 + 0.7 ≧ 4.6)

| | | |
|---|---|---|
| Proton pump inhibitor: | 500 | mg (a range of |
| Omeprazole powder | | 350 to 700 mg) |
| Primary Essential Buffer(s): | | |
| Sodium bicarbonate | 5 | g (59.5 mEq) |
| Dibasic sodium phosphate | 2 | g (14 mEq) |
| (anhydrous) | | |
| Optional Secondary Essential Buffer(s): | | |
| Tribasic sodium phosphate | 200 | mg. (1.2 mEq) |

(* Any Secondary Essential Buffer(s) may be added in higher or lower amounts to adjust pH for desired stability and additive antacid or buffering effect.)

Powders of the above compounds are combined as is known in the art to create a homogenous mixture with the addition of a thickener such as guar gum 350 mg, artificial maple flavor powder 100 mg, thaumatin powder 10 mg (to mask the bitterness of omeprazole), and sucrose 25 Gm. Q.s. to 100 mL with distilled water to achieve a final omeprazole concentration of 5 mg/mL. Different volumes of water may be added to achieve omeprazole concentrations ranging from about 0.8 to about 20 mg/mL.

Alternatively, this formulation may be divided into two parts. The dry part may be reconstituted with the liquid part at the time of use.

Formulation 6: Veterinary Formulation of Lansoprazole

| Essential pH (lansoprazole pKa = 4.1 + 0.7 ≧ 4.8) | | |
|---|---|---|
| EBC = 71.4 mEq | | |
| Proton pump inhibitor: | 750 | mg |
| Lansoprazole powder | | |
| Primary Essential Buffer(s): | | |
| Sodium bicarbonate | 6 | g (71.4 mEq) |

(* Any Secondary Essential Buffer(s) may be added in higher or lower amounts to adjust pH for desired stability and additive antacid or buffering effect.)

Powders of the above compounds are combined as is known in the art to create a homogenous mixture with the addition of a thickener such as xanthan gum 300 mg, artificial peanut butter flavor powder 100 mg, and sucrose 35 Gm. Q.s. to 100 mL with distilled water to achieve a final lansoprazole concentration of 7.5 mg/mL. The suspension should be refrigerated after reconstitution. Different volumes of water may be added to achieve lansoprazole concentrations ranging from 0.8 to 20 mg/mL.

Alternatively, this formulation may divided into two parts. The dry part may be reconstituted with the liquid part at the time of use.

Formulation 7: Veterinary Formulation of Lansoprazole

| Essential pH (lansoprazole pKa = 4.1 + 0.7 ≧ 4.8) | | |
|---|---|---|
| EBC = 63.3 mEq | | |
| Proton pump inhibitor: | | |
| Lansoprazole powder | 750 | mg |
| Primary Essential Buffer(s) | | |
| Sodium bicarbonate | 5 | g (59.5 mEq) |
| Secondary Essential Buffer(s): | | |
| Sodium carbonate | 400 | mg* (3.8 mEq) |

(*Any Secondary Essential Buffer(s) may be added to adjust pH for desired stability and additive antacid or buffering effect.)

Powders of the above compounds are combined as is known in the art to create a homogenous mixture with the addition of a thickener such as hydroxypropyl methyl cellulose 300 mg, artificial maple flavor 100 mg, and sucrose 35 Gm. Q.s. to 100 mL with distilled water to achieve a final lansoprazole concentration of 7.5 mg/mL. Different volumes of water may be added to achieve lansoprazole concentrations ranging from 0.3 to 20 mg/mL.

Alternatively, this formulation may divided into two parts. The dry part may be reconstituted with the liquid part at the time of use.

Formulation 8: Veterinary Formulation
of Esomeprazole Magnesium

| Essential pH (esomeprazole pKa = 3.9 + 0.7 ≧ 4.6) | | |
|---|---|---|
| EBC = 53.2 mEq | | |
| Proton pump inhibitor: | | |
| Esomeprazole magnesium powder | 500 | mg |
| Primary Essential Buffer(s): | | |
| Sodium bicarbonate | 5 | g (47.6 mEq) |
| Dibasic sodium phosphate | 800 | mg (5.6 mEq) |

(* Any Secondary Essential Buffer(s) may be added in higher or lower amounts to adjust pH for desired stability and additive antacid or buffering capacity.)

Powders of the above compounds are combined as is known in the art to create a homogenous mixture with the addition of a thickener such as hydroxypropyl cellulose 300 mg, artificial butterscotch flavor 100 mg, thaumatin powder 5 mg, and sucrose 30 Gm. Q.s. to 100 mL with distilled water to achieve a final esomeprazole concentration of 7.5 mg/mL. Different volumes of water may be added to achieve esomeprazole concentrations ranging from 0.8 to 20 mg/mL.

Formulation 9: Veterinary Formulation of Pantoprazole
Sodium or Pantoprazole Base ™ Powder

| Essential pH (pantoprazole sodium pKa = 3 + 0.7 ≧ 3.7) | | |
|---|---|---|
| EBC = 53.8 mEq | | |
| Pantoprazole sodium or | 1000 | mg |
| pantoprazole powder | | |
| Primary Essential Buffer(s): | | |
| Sodium bicarbonate | 4 | g (47.6 mEq) |
| Secondary Essential Buffer(s): | | |
| Trisodium phosphate | 1000 | mg* (6.2 mEq) |

(*Any Secondary Essential Buffer(s) may be added in higher or lower amounts to adjust pH for desired stability and additive antacid or buffering capacity.)

Powders of the above compounds are combined as is known in the art to create a homogenous mixture with the addition of a thickener such as hydroxypropyl cellulose 300 mg, artificial butterscotch flavor 100 mg, thaumatin powder 5 mg, and sucrose 30 Gm. Q.s. to 100 mL with distilled water to achieve a final pantoprazole concentration of 10 mg/mL. Different volumes of water may be added to achieve esomeprazole concentrations ranging from 0.2 to 20 mg/mL.

Formulation 10: Veterinary Formulation: Buffer
Base ™ Without Proton Pump Inhibitor

| EBC = 71.4 mEq | | |
|---|---|---|
| Primary Essential Buffer: | | |
| Sodium bicarbonate | 6 | g 71.4 mEq |
| Optional Secondary Essential Buffer: | | |
| Tribasic sodium phosphate | 1000 | mg* |

(*Any Secondary Essential Buffer may be added in higher or lower amounts to adjust pH for desired stability and additive antacid or buffering capacity.)

Powders of the above compounds are combined as is known in the art to create a homogenous mixture with the addition of a thickener such as hydroxypropyl cellulose 300 mg, artificial butterscotch flavor 100 mg, thaumatin powder 5 mg, and sucrose 30 Gm. Q.s. to 100 mL with distilled water. A proton pump inhibitor or other acid-labile drug may be added by the compounding pharmacist selected from available proton pump inhibiting agents or acid-labile drugs from powder or enteric-coated oral solid dosage forms. Different volumes of water may be added to achieve proton pump inhibitor concentrations ranging from 0.8 to 20 mg/mL. If other acid labile drugs are employed, the range of concentrations would be as required to deliver the normal dosage in an acceptable volume of 1 mL to 30 mL. The amount of buffer required to protect the drug in question will also determine the minimal feasible volume. This formulation may be in the form of a one-part product (liquid or dry) or a two-part product (liquid and dry), for examples. In the two-part example, the drug to be added to the formulation may be added to the dry formulation and the liquid part may be added at the time of use, or the drug may be added to the liquid portion which would be buffered to a pH above that required for disintegration of enteric-coated drug formulations (typically pH of 6.8 or greater).

For all of the veterinary and human oral dosage forms disclosed herein, sweeteners, parietal cell activators, thickeners, preservatives, and flavoring agents may also be added. Sweeteners include but are not limited to corn syrup, simple syrup, sugar, thaumatin, and aspartame. Thickeners include but are not limited to methylcellulose, xanthan gum, carrageenan, and guar gum. Preservatives may be added to retard spoilage and include but are not limited to sodium benzoate, methylparaben and propylparaben. Flavoring agents in these formulations include but are not limited to apple, caramel, maple, peanut butter, meat, etc.

P. Other Formulations

For all formulations herein, the total amount of Essential Buffer may range from about 4 mEq to about 30 mEq per dose.

Formulation 11: Oral Buffer Complex Without Proton
Pump Inhibitor (for general use to protect acid
labile drugs) Multidose Composition

| Primary Essential Buffer: | | |
|---|---|---|
| Dibasic sodium phosphate or | 10 | g |
| sodium bicarbonate | (range 2 | g to 10 g) |
| Optional Secondary Essential Buffer: | | |
| Tribasic sodium phosphate or | 200 | mg |
| sodium carbonate | | |
| Other ingredients: | | |
| Sucrose | 26 | g |
| Maltodextrin | 2 | g |
| Cocoa processed with alkaline | 1800 | mg |
| Corn syrup solids | 6000 | mg |
| Sodium caseinate | 100 | mg |
| Soy lecithin | 80 | mg |

(* Any Secondary Essential Buffer may be added in higher or lower amounts to adjust pH for desired stability and additive antacid or buffering capacity.)

Thoroughly blend the powder, then store in a container protected from light and moisture, such as in a foil packet. Preservatives may be added to retard spoilage and include but are not limited to sodium benzoate, methylparaben, and propylparaben. Thickeners such as xanthan gum, guar gum, or hydroxymethyl propyl cellulose can be flavoring agents in these formulations include chocolate, caramel, maple, butter pecan and other flavorings as have been outlined previously. Different volumes of water may be added to achieve proton pump inhibitor concentrations ranging from 0.8 to 20 mg/mL.

Weigh out approximately 60 g of the formulation. Add proton pump inhibitor (or other acid-labile drug) typically in the amount equivalent to 10 doses (range 1 dose to 30 doses). Q.s. to 100 mL with distilled water.

Formulation 12: Oral Buffer Complex Without Proton
Pump Inhibitor For General Use to Protect Acid Labile
Drugs; Protein Free, Multi-Dose Example

| Primary Essential Buffer: | | |
|---|---|---|
| Sodium bicarbonate | 5 | g |
| | (range 2 | g to 10 g) |
| | (59.5 | mEq) |
| Optional: Secondary Essential Buffer | | |
| None* | | |
| Other ingredients | | |
| Sucrose | 26 | g |
| Maltodextrin | 2 | g |
| Cocoa processed with alkaline | 1800 | mg |
| Corn syrup solids | 6000 | mg |
| Soy lecithin | 80 | mg |

(*Any Secondary Essential Buffer may be added in higher or lower amounts to adjust pH for desired stability and additive antacid or buffering capacity.)
Note that cocoa is a parietal cell activator.

Thoroughly blend the powder, then store in a container protected from light and moisture, such as in a foil packet. Weigh out approximately 60 g of the formulation. Add proton pump inhibitor (or other acid-labile drug) typically in the amount equivalent to 10 doses (range=1 dose to 30 doses).

Q.s. to 100 mL with distilled water. Different volumes of water may be added to achieve proton pump inhibitor concentrations ranging from 0.8 to 20 mg/mL.

Formulation 13: Buffer Complex Without Proton Pump
Inhibitor For General Use to Protect Acid Labile Drugs;
Protein Free, Lactose Free Multidose Example

| Proton pump inhibitor: | | |
|---|---|---|
| None (to be added later, e.g. by | | |
| compounding pharmacist) | | |
| Primary Essential Buffer(s): | | |
| Sodium bicarbonate | 8 | g |
| | (range 2 | g to 10 g) |
| Other ingredients: | | |
| Sucrose | 26 | g |
| Maltodextrin | 2 | g |
| Corn syrup solids | 6000 | mg |
| Partially hydrogenated soybean oil | 400 | mg |
| Dipotassium phosphate | 300 | mg |
| Caramel flavor | 270 | mg |
| Soy lecithin | 80 | mg |
| Sodium silico aluminate | 20 | mg |
| Titanium dioxide | 10 | mg |

Thoroughly blend the powder, then store in a container protected from light and moisture, such as in a foil packet. Optional Secondary Essential Buffer: Tribasic sodium phosphate 1000 mg

Weigh out approximately 60 g of the formulation. Add proton pump inhibitor (or other acid-labile drug) typically in the amount equivalent to 10 doses (range=1 dose to 30 doses). Q.s. to 100 mL with distilled water. Different volumes of water may be added to achieve proton pump inhibitor concentrations ranging from 0.3 to 20 mg/mL.

Formulation 14: Buffer Complex Without Proton Pump
Inhibitor For General Use to Protect Acid Labile
Drugs; Protein Free, Multi-Dose Example

| Proton pump inhibitor: | | |
|---|---|---|
| None (to be added later, e.g. by | | |
| compounding pharmacist) | | |
| Primary Essential Buffer(s): | | |
| Dibasic sodium phosphate | 8 | g |
| | (range 2 | g to 10 g) |
| Other ingredients: | | |
| Sucrose | 26 | g |
| Maltodextrin | 2 | g |
| Butterscotch flavor | 270 | mg |
| Corn syrup solids | 6000 | mg |

Thoroughly blend the powder, then store in a container protected from light and moisture, such as in a foil packet.

Weigh out approximately 60 g of the formulation. Add proton pump inhibitor (or other acid-labile drug) typically in the amount equivalent to 10 doses (range=1 dose to 30 doses). Q.s. to 100 mL with distilled water. Different volumes of water may be added to achieve proton pump inhibitor concentrations ranging from 0.8 to 20 mg/mL.

Formulation 15: Buffer Complex Without Proton Pump
Inhibitor For General Use to Protect Acid Labile
Drugs; Protein Free, Multi-Dose Example

| Proton pump inhibitor: | | |
|---|---|---|
| None (to be added later, e.g. by | | |
| compounding pharmacist) | | |
| Primary Essential Buffer(s): | | |
| Sodium bicarbonate | 8 | g |
| | (range 1 | g to 10 g) |
| Secondary Essential Buffer(s): | | |
| Trisodium phosphate | 1.5 | g |
| | (range 0 | g to 5 g) |
| Other ingredients: | | |
| Sucrose | 26 | g |
| Maltodextrin | 2 | g |
| Butterscotch flavor | 270 | mg |
| Corn syrup solids | 6000 | mg |

Thoroughly blend the powder, then store in a container protected from light and moisture, such as in a foil packet. Weigh out approximately 60 g of the formulation. Add proton pump inhibitor (or other acid-labile drug) typically in the amount equivalent to 10 doses (range=1 dose to 30 doses). Q.s. to 100 mL with distilled water. Different volumes of water may be added to achieve proton pump inhibitor concentrations ranging from 0.8 to 20 mg/mL.

Formulation 16: One Phase Lansoprazole 30 mg Tablet

Lansoprazole has a pKa of 4.1; thus, the Essential pH = 4.1 + 0.7 ≧ 4.8
Examples of buffers that produce a solution with pH 4.8 or greater and
produce the Essential Buffering Capacity include, but are not limited
to, sodium bicarbonate, sodium carbonate, dibasic sodium phosphate,
and dipotassium phosphate.
Enough powder for 11 tablets is weighed out:

| | |
|---|---|
| Proton pump inhibitor: | |
| Lansoprazole powder | 330 mg |
| Primary Essential Buffer(s): | |
| Sodium bicarbonate USP | 5500 mg |
| Dibasic sodium phosphate | 2200 mg |

The resultant powder is thoroughly mixed. Then 720 mg of the homogeneous mixture is poured into a tablet reservoir (½ inch diameter) and pressed through a full motion of the press as is known in the art. The resultant tablet contains:

| | |
|---|---|
| Lansoprazole | 30 mg |
| Sodium bicarbonate USP | 500 mg |
| Disodium hydrogen phosphate | 200 mg |

The tablet contains 6 mEq sodium bicarbonate and 1.4 mEq dibasic sodium phosphate. Variations in this tablet may include a tablet containing all dibasic sodium phosphate or all sodium bicarbonate or other buffers from the Essential Buffers list. The amount of Effective Buffer Capacity per tablet may range from as little as about 4 mEq to as much as about 30 mEq.

Additional tablet disintegrants such as croscarmelose sodium, pregelatinized starch, or providone, and tablet binders such as tapioca, gelatin, or PVP may be added. Further, a film coating may be placed on the tablet to reduce the penetration of light and improve ease of swallowing.

Formulation 17: One Phase Omeprazole 20 mg Tablet

Omeprazole has a pKa of 3.9; thus, the Essential pH = 3.9 + 0.7 ≧ 4.6
Examples of buffers that are soluble at pH 4.6 or greater include,
but are not limited to, sodium bicarbonate, sodium carbonate,
disodium hydrogen phosphate (dibasic sodium phosphate), and
dipotassium phosphate.
Enough powder for 11 tablets is weighed out:

| | |
|---|---|
| Proton pump inhibitor: | |
| Omeprazole powder USP | 220 mg |
| Primary Essential Buffer(s): | |
| Sodium bicarbonate USP | 6500 mg |
| Magnesium oxide powder | 1650 mg |
| Croscarmelose sodium | 300 mg |

The resultant powder is thoroughly mixed. Then 788 mg of the homogeneous mixture is poured into a tablet reservoir (½ inch diameter) and pressed through a full motion of the press as is known in the art. The resultant tablet contains:

| | |
|---|---|
| Omeprazole USP | 20 mg |
| Sodium bicarbonate USP | 590 mg |
| Magnesium oxide | 150 mg |
| Croscarmelose sodium | 27.27 mg |

The tablet contains 7 mEq sodium bicarbonate and 3.75 mEq magnesium oxide. The amount of Effective Buffer Capacity may range from as little as about 4 mEq to as much as about 30 mEq. The tablet excipients, tablet binders, and film coating of Formulation 16 may also be added.

Formulation 18: One Phase Omeprazole 40 mg Tablet

| Enough powder for 11 tablets is weighed out: | |
|---|---|
| Proton pump inhibitor: | |
| Omeprazole powder USP | 440 mg |
| Primary Essential Buffer(s): | |
| Sodium bicarbonate USP | 6500 mg |
| Magnesium oxide | 1650 mg |
| Pregelatinized starch | 500 mg |

The resultant powder is thoroughly mixed. Then 826 mg of the homogeneous mixture is poured into a tablet reservoir (½ inch diameter) and pressed through a full motion of the press as is known in the art. The resultant tablet contains:

| | |
|---|---|
| Omeprazole USP | 40 mg |
| Sodium bicarbonate USP | 590 mg |
| Magnesium oxide | 150 mg |
| Pregelatinized starch | 45.45 mg |

The tablet contains 7 mEq sodium bicarbonate and 3.75 mEq magnesium oxide. The amount of Effective Buffer Capacity may range from as little as 4 mEq to as much as 30 mEq. The tablet excipients, tablet binders, and film coating of Formulation 16 may also be added.

Esomeprazole magnesium or other proton pump inhibiting agents which are of low solubility (such as the base forms) may be used in place of omeprazole or lansoprazole in the above formulations. The tablet excipients, tablet binders, and film coatings of Formulation 16 may also be added. In addition, powders of any of the formulations disclosed herein may be manufactured by thoroughly mixing the powders as when making tablets and omitting the pressing of the tablets. The powder is packaged in a suitable container protecting the formulation from air moisture and light such as a foil pack or sachet. When added to a volume of water (e.g. 3 to 20 mL) the formulation may be taken orally or administered down a feeding or NG tube, etc. Flavoring agents such as are outlined in the above formulations may be used, for example, carmel flavor 0.1 % w/w. For bitter tasting proton pump inhibiting agents such as pantoprazole, omeprazole, esomperazole and rabeprazole, the use of thaumatin in a quantity of 5 to 10 ppm may be useful in masking the bitterness. Sweeteners such as sucrose or aspartame may also be employed. Tablet disintegrants such as croscarmelose sodium and glidants such as magnesium stearate may additionally be used.

Formulation 19: Omeprazole Powder Formulations (single dose)

| | | |
|---|---|---|
| Proton pump inhibitor: | | |
| Omeprazole powder USP | 20 | mg or |
| (or esomeprazole magnesium). | 40 | mg |
| Primary Essential Buffer(s): | | |
| Sodium bicarbonate USP powder (60 micron) | 1000 | mg |
| Magnesium oxide USP powder | 500 | mg |
| Optional Secondary Essential Buffer(s): | | |
| Tribasic sodium phosphate | 200 | mg* |
| Other ingredients: | | |
| Dextrose | 60 | mg |
| Xanthan gum (Rhodigel ultra fine) | 15 | mg |
| Thaumatin (Flavor enhancer) | 5 to 10 | ppm |

Thoroughly blend the powder, reconstitute all of the powder with 5 ml to 20 ml distilled water and administer the suspension enterally to the patient.

Formulation 20: Unflavored Omeprazole Powder (single dose)

| | | |
|---|---|---|
| Omeprazole powder USP | 20 | mg or |
| | 40 | mg |
| Sodium bicarbonate USP | 1500 | mg |
| Parietal cell activator: | | |
| Calcium chloride | 200 | mg |
| Other ingredients: | | |
| Dextrose | 60 | mg |
| Xanthan gum (Rhodigel ulta fine) | 15 | mg |
| Thaumatin (Flavor enhancer) | 5 to 10 | ppm |

Thoroughly blend the powder. Reconstitute all of the powder with 5 mL to 20 mL distilled water and administer the suspension enterally to the patient.

Formulation 21: Flavored Omeprazole Powder (single dose)

| | | |
|---|---|---|
| Omeprazole powder USP | 20 | mg |
| Dibasic sodium Phosphate duohydrate | 2000 | mg |
| Sodium bicarbonate USP | 840 | mg to |
| | 1680 | mg |
| Sucrose | 2.6 | g |
| Maltodextrin | 200 | mg |
| Cocoa processed with alkaline* | 180 | mg |
| Corn syrup solids | 600 | mg |
| Xanthan gum | 15 | mg |
| Aspartame | 15 | mg |
| Thaumatin | 2 | mg |
| Soy lecithin | 10 | mg |

*Parietal cell activator

Thoroughly blend the powder. Reconstitute all of the powder with 10 mL to 20 mL distilled water at the time of use.

Formulation 22: Unflavored Lansoprazole Powder (single dose)

| | |
|---|---|
| Lansoprazole powder USP | 15 mg or 30 mg |
| Sodium bicarbonate USP | 400 mg to 1500 mg |

Optionally: Tribasic sodium phosphate to adjust pH for longer stability and enhanced buffering capacity (alternatively other Essential Buffers may be employed)

Thoroughly blend the powder. Reconstitute all of the powder with 5 mL to 20 mL distilled water at the time of use.

Formulation 23: Flavored Lansoprazole Powder (single dose)

| Proton pump inhibitor: | | |
|---|---|---|
| Lansoprazole powder USP | 30 | mg |
| Primary Essential Buffer(s): | | |
| Dibasic Sodium Phosphate USP or | 1500 | mg |
| Sodium bicarbonate USP | | |
| Sucrose | 26 | g |
| Maltodextrin | 2 | g |
| Cocoa processed with alkaline* | 18 | mg |
| Corn syrup solids | 600 | mg |
| Soy lecithin | 80 | mg |

*Parietal cell activator

Thoroughly blend the powder. Reconstitute all of the powder with 5 mL to 20 mL distilled water at the time of use.

Formulation 24: Unflavored Rabeprazole Powder (single dose)

| | |
|---|---|
| Proton pump inhibitor: | |
| Rabeprazole sodium powder USP | 20 mg |
| Primary Essential Buffer(s): | |
| Disodium phosphate duohydrate USP | 2000 mg |
| Optional Secondary Essential Buffer(s) | |
| Tribasic sodium phosphate | 100 mg |

Thoroughly blend the powder and reconstitute with distilled water prior to administration. Optionally, thickeners and flavoring agents may be added as stated throughout this application. The anticipated volume for this powder would be 20 mL per dose. This formulation is designed to enhance stability of rabeprazole through the use of the common ion effect whereby sodium causes a “salting out” of rabeprazole sodium. This causes the rabeprazole sodium to remain insoluble thereby increasing its stability.

Formulation 25: Unflavored Rabeprazole Powder (single dose)

| | |
|---|---|
| Proton pump inhibitor: | |
| Rabeprazole sodium powder USP | 20 mg |
| Primary Essential Buffer(s): | |
| Sodium bicarbonate USP | 1200 mg |
| Secondary Essential Buffer(s): | |
| Trisodium phosphate USP | 300 mg |
| Optional Secondary Essential Buffer(s): | |

Sodium hydroxide or Tribasic potassium may be added in higher or lower
amounts to adjust pH for desired stability and additive antacid or
buffering capacity.

Thoroughly blend the powder and reconstitute with 15 mL distilled water at the time of use.

Alternatively, a two part product may be employed comprising one part of about 5 to about 15 mL distilled water with a low concentration of Secondary Essential Buffer (e.g. trisodium phosphate (100 mg) or sodium hydroxide (50 mg)) used to dissolve an enteric-coated tablet of rabeprazole thereby producing a stable solution/suspension. This highly alkaline suspension containing low neutralization capacity and rabeprazole sodium may then be added with a second part containing the Primary Essential Buffer(s) having significant neutralization capacity. If desired other Secondary Essential Buffer(s) may be included with the Primary Essential Buffers. This formulation is designed to enable the use of the commercially available enteric-coated tablet of rabeprazole as the source of the proton pump inhibitor. This tablet requires disintegration prior to use as a liquid formulation. Part 1 (the low concentration of Secondary Essential Buffer) produces rapid dissolution of the delayed-release tablet as well as prolonged stability of rabeprazole sodium in the liquid form. This enables the preparation to be prepared prior to administration and simply added to the Primary Essential Buffer(s) (part 2) prior to use.

Formulation 26: Unflavored Rabeprazole Powder (single dose)

| | |
|---|---|
| Proton pump inhibitor: | |
| Rabeprazole sodium powder USP | 20 mg |
| Primary Essential Buffer(s): | |
| Calcium lactate USP | 700 mg |
| Calcium glycerophosphate | 700 mg |
| Secondary Essential Buffer(s): | |
| Calcium hydroxide USP | 15 mg |

(Other Secondary Essential Buffers with cations of sodium or potassium may be added in higher or lower amounts to adjust pH for desirable stability.)

Thoroughly blend the powder. Reconstitute the powder with a liquid part comprising 10 mL glycerol and 10 mL distilled water at the time of use. Alternatively, the liquid for reconstitution may be only water (e.g. distilled) and contain some of the buffer. The liquid for reconstitution may be supplied as a buffered product (to pH 9-11) for dissolving rabeprazole sodium delayed-release tablets (if used as a source of rabeprazole sodium).

Formulation 27: Unflavored Esomeprazole Powder (single dose)

| | |
|---|---|
| Proton pump inhibitor: | |
| Esomeprazole magnesium powder USP | 20 mg |
| Primary Essential Buffer(s): | |
| Calcium lactate USP | 800 mg |
| Calcium glycerophosphate | 800 mg |
| Secondary Essential Buffer(s): | |
| Calcium hydroxide USP | 15 mg |

(Other Secondary Essential Buffers with cations of calcium or magnesium may be added in higher or lower amounts to adjust pH for desirable stability.)

Thoroughly blend the powder. Reconstitute the powder with a liquid part comprising of 10 mL distilled water at the time of use. The liquid for reconstitution may be supplied as a buffered product (to pH 8-11) for dissolving esomeprazole magnesium delayed release granules (if used as a source of esomeprazole magnesium).

Formulation 28: Omeprazole Two Part Tablet

Two part tablets contain an outer buffer phase and inner buffer/
Proton pump inhibitor core. Enough for 6 tablets is weighed out.

| | |
|---|---|
| Inner Core: | |
| Proton pump inhibitor: | |
| Omeprazole powder USP | 120 mg |
| (or esomeprazole magnesium or omeprazole sodium). | |
| Primary Essential Buffer(s): | |
| Sodium bicarbonate USP | 1200 mg |
| Outer Phase: | |
| Sodium bicarbonate USP | 3960 mg |

(Secondary Essential Buffers such as trisodium phosphate, tripotassium phosphate or sodium carbonate or others may be added to enhance neutralization capacity.)

Thoroughly blend the powders for the inner core, then weigh out approximately 220 mg of the resultant blend and add to a die of ⅜″ diameter. The powder mixture is then formulated into small tablets by conventional pharmaceutical procedures. Repeat for five additional tablets, then set these small inner tablets aside.

The outside layer surrounding the proton pump inhibitor tablet serves as a pH-buffering zone. Enough sodium bicarbonate for 6 tablets is weighed out with approximately 280 mg per tablet for a total of 1680 mg sodium bicarbonate USP. Then weigh out approximately 280 mg of the resultant blend and add to a die of ½″ diameter. Press through a full motion to compact the powder into a tablet. Place the tablet back into the ½ inch die and then place the smaller ⅜″ tablet (inner tablet) on top of the ½ tablet and center it. Add approximately 380 mg sodium bicarbonate to the die on top of the ½″ tablet and the ⅜″ tablet. Press through a full motion to compact the materials into one tablet. The approximate weight of each tablet is 815 mg to 890 mg containing 20 mg omeprazole. Binders such as tapioca or PVP and disintigrants such as pregelatinized starch may be added. The outer lay may also comprise pharmaceutically acceptable tablet exipients. Optional coatings can also be employed, for example, light film coatings and coatings to repel ultraviolet light as is known in the art.

Magnesium oxide or magnesium hydroxide may be substituted for the sodium bicarbonate outer phase. Enough magnesium oxide for 6 tablets is weighed out with approximately 280 mg per tablet for a total of 1680 mg magnesium oxide USP. Then weigh out approximately 280 mg of the resultant blend and add to a die of ½″ diameter. Press through a full motion to compact the powder into a tablet. Place the tablet back into the ½ inch die and then place the smaller ⅜″ tablet (inner tablet) on top of the ½″ tablet and center it. Add approximately 380 mg magnesium oxide to the die on top of the ½″ tablet and the ⅜″ tablet. Press through a full motion to compact the materials into one tablet. The approximate weight of each tablet is 815 mg to 890 mg containing 20 mg omeprazole. Binders such as tapioca or PVP and disintigrants such as pregelatinized starch, croscarmelose sodium or microcrystalline cellulose (MCC) and colloidal silicone dioxide (CSD) may be added. The outer layer may also comprise pharmaceutically acceptable tablet exipients. Optional coatings can also be employed, for example, light film coatings and coatings to repel ultraviolet light as is known in the art.

The outer phase can alternatively comprise a combination of sodium bicarbonate and magnesium oxide.

Formulation 29: Lansoprazole Two Part Tablet

| Enough for 6 tablets is weighed out. | | |
|---|---|---|
| Inner Core: | | |
| Proton pump inhibitor: | | |
| Lansoprazole powder USP | 180 | mg |
| Primary Essential Buffer: | | |
| Sodium bicarbonate USP | 1200 | mg |
| Outer Phase: | | |
| Sodium bicarbonate USP | 3960 | mg |

Thoroughly blend the powders of the inner core, then weigh out approximately 230 mg of the resultant blend and add to a die of ⅜″ diameter. The inner and outer tablets are then formed as described in Formulation 28. The approximate weight of each tablet is 825 mg to 900 mg. Binders such as tapioca or PVP and disintigrants such as pregelatinized starch may be added.

Formulation 30: Pantoprazole Two Part Tablet

| Enough for 6 tablets is weighed out. | | |
|---|---|---|
| Inner Core: | | |
| Proton pump inhibitor: | | |
| Pantoprazole powder USP | 240 | mg |
| (or pantoprazole sodium) | | |
| Primary Essential Buffer: | | |
| Sodium bicarbonate USP | 1200 | mg |
| Outer Phase: | | |
| Sodium bicarbonate USP | 3960 | mg |

Thoroughly blend the powders for the inner core, then weigh out approximately 220 mg of the resultant blend and add to a die of ⅜″ diameter. The inner and outer tablets are then formed as described in Formulation 28. The approximate weight of each tablet is 835 mg to 910 mg. Binders such as tapioca or PVP and disintigrants such as pregelatinized starch or croscarmelose sodium may be added.

Formulation 31: Omeprazole or esomeprazole two part tablet.

| Enough for 6 tablets is weighed out. | | |
|---|---|---|
| Inner Core: | | |
| Proton pump inhibitor: | | |
| Omeprazole powder USP (or esomeprazole or | 120 | mg |
| omeprazole sodium). | | |
| Primary Essential Buffer: | | |
| Sodium bicarbonate | 1200 | mg |
| Outer Phase: | | |
| Sodium bicarbonate | 3960 | mg |

Thoroughly blend the powders of the inner core, then weigh out approximately 220 mg of the resultant blend and add to a die of ⅜″ diameter. The inner and outer tablets are then formed as described in Formulation 28. The approximate weight of each tablet is 815 mg to 890 mg. Binders such as tapioca or PVP and disintigrants have been mentioned and may be added. Secondary Essential Buffers such as trisodium phosphate, tripotassium phosphate or sodium carbonate or others may be added to enhance neutralization capacity.

Formulation 32: Lansoprazole Two part tablet

| Enough for 6 tablets is weighed out. | | |
|---|---|---|
| Inner Core: | | |
| Proton pump inhibitor: | | |
| Lansoprazole powder USP | 180 | mg |
| Primary Essential Buffer: | | |
| Sodium bicarbonate | 1200 | mg |
| Outer Phase: | | |
| Sodium bicarbonate | 3960 | mg |

Thoroughly blend the powder of the inner core, then weigh out approximately 230 mg of the resultant blend and add to a die of ⅜″ diameter. The inner and outer tablets are then formed as described in Formulation 28. The approximate weight of each tablet is 825 mg to 900 mg. Binders such as tapioca or PVP and disintigrants have been mentioned and may be added. Secondary Essential Buffers such as trisodium phosphate, tripotassium phosphate or sodium carbonate or others may be added to enhance neutralization capacity.

Formulation 33: Pantoprazole Two part tablet

| Enough for 6 tablets is weighed out. | | |
|---|---|---|
| Inner Core: | | |
| Proton pump inhibitor: | | |
| Pantoprazole sodium powder USP | 240 | mg |
| Primary Essential Buffer: | | |
| Sodium bicarbonate | 1200 | mg |
| Outer Phase: | | |
| Sodium bicarbonate | 3960 | mg |

Thoroughly blend the powders of the inner core, then weigh out approximately 220 mg of the resultant blend and add to a die of ⅜″ diameter. The inner and outer tablets are then formed as described in Formulation 28. The approximate weight of each tablet is 835 mg to 910 mg. Binders such as tapioca or PVP and disintegrants may also be added. Secondary Essential Buffers, such as trisodium phosphate, tripotassium phosphate, sodium carbonate or others, may be added to enhance neutralization capacity.

Formulation 34: Omeprazole 20 mg Two-Part Tablet

| Inner Core: | | |
|---|---|---|
| Proton pump inhibitor: | | |
| Omeprazole enteric coated granules (base, or | 20 | mg |
| sodium salt or esomeprazole sodium or magnesium) | | |
| Outer Phase: | | |
| Sodium bicarbonate powder USP | 1000 | mg |

The inner core is created as is known in the art such that the enteric coatings on the granules remain substantially intact. The outer phase is bound to the inner core as described in Formulation 28. Other variations of this tablet include a uniform enteric coating surrounding the proton pump inhibitor of the inner core instead of separate enteric coated granules.

Formulation 35: Lansoprazole 30 mg Two-Part Tablet

| Inner Core: | | |
|---|---|---|
| Proton pump inhibitor: | | |
| Lansoprazole enteric coated granules | 30 | mg |
| Outer Phase: | | |
| Sodium bicarbonate powder USP | 1000 | mg |

This two-part tablet is formulated as per Formulation 34.

Formulation 36: Rabeprazole 20 mg Two-Part Tablet

| Inner Core: | | |
|---|---|---|
| Proton pump inhibitor: | | |
| Rabeprazole enteric coated granules | 20 | mg |
| Outer Phase: | | |
| Sodium bicarbonate powder USP | 1000 | mg |

This two-part tablet is formulated as per Formulation 34.

Formulation 37: Omeprazole Two Part Tablet

| Enough for 6 tablets is weighed out | | |
|---|---|---|
| Inner Core: | | |
| Omeprazole | 120 | mg |
| Sodium bicarbonate power USP | 1200 | mg |
| Outer Phase: | | |
| Magnesium oxide | 1500 | mg |
| Optional - calcium carbonate | 3000 | mg |

The omeprazole and sodium bicarbonate of the inner core are homogeneously mixed and formed as in Formulation 28. The outer phase is combined with the inner core as in Formulation 28.

Formulation 38: Combination Antacid
and Enteric Coated Dosage Form

| | | |
|---|---|---|
| Omeprazole enteric | 20 | mg (or an equivalent dose of |
| coated granules or | | another proton pump inhibitor) |
| enteric coated tablet | | |
| Calcium carbonate | 1000 | mg |

The above components are combined with care exerted to ensure that the enteric coating is not crushed or otherwise compromised. The resulting combination is then formed into compressed tablets or placed in capsules as is known in the pharmaceutical art. If enteric coated granules are employed, they are generally, but not required, dispersed throughout the tablet or capsule. If an enteric coated tablet is alternatively utilized, it forms a central core, which is uniformly surrounded by the calcium carbonate in either a compressed tablet or in a larger capsule. In another embodiment, a capsule containing enteric coated granules of proton pump inhibitor can be placed within a larger capsule containing the calcium carbonate.

It should be noted that other buffering agents can be utilized in lieu of or in combination with calcium carbonate. The buffer(s) employed is present in an amount of at least about 5 mEq per dose of the composition with the preferred range been 7.5 to 15 mEq. For example, sodium bicarbonate may be preferred over calcium carbonate and other antacids (such as magnesium or aluminum salts) because in many cases, sodium bicarbonate more quickly lowers gastric pH.

Formulation 39: Combination Rapid Release and Delayed
Released Proton Pump Inhibitor and Antacid

| Inner core: | | |
|---|---|---|
| Omeprazole enteric coated | 10 or 20 | mg (or an equivalent dose of |
| granules or enteric | | another proton pump inhibitor) |
| coated tablet | | |
| Outer phase: | | |
| Omeprazole powder | 10 or 20 | mg (or equivalent dose of |
| | | another proton pump inhibitor) |
| Calcium Carbonate powder | 1000 | mg |

The constituents of the outer phase are uniformly mixed. The inner core is created as is known in the art such that the enteric coatings on the granules or tablet remain substantially intact. The outer phase is bound to the inner core as described herein and as known in the art.

Formulation 40: Soft Chewable Proton Pump Inhibitor-Buffer Dosage Form

Omeprazole 10 or 20 mg (or an equivalent dose of another proton pump inhibitor) is combined with the ingredients of a soft chewable antacid tablet (e.g., Viactiv®), which comprises calcium carbonate 500 or 1000 mg, corn syrup, sugar, chocolate non fat milk, cocoa butter, salt, soy lecithin, glyceryl monostearate, flavoring (e.g., caramel), carrageenan, and sodium phosphate. Vitamins D3 and/or K1 can also be added. The finished chew tablets are administered to patients once to thrice daily for gastric acid related disorders.

### Example XVII

An in vitro Kinetic Acid Neutralization experiment was performed to evaluate impact of various amounts of buffering agent on simulated stomach. For this experiment, one hundred ml of 0.1 N HCl was placed into a stomach beaker with stirring and maintained at 37° C. A perfusion pump equiped with 2 heads was set up so that 0.1 N HCL from a feeder beaker could be pumped into the stomach beaker while the same volume of liquid from the stomach beaker could be simultaneously pumped out, thereby simulating stomach emptying. It is assumed that an average empty stomach has 50 ml of acid, however, for experimantal convenience, one hundred ml of liquid was used (thereby doubling the empty stomach acid volume). All other volumes and/or concentrations were doubled correspondingly.

The experiment assessed the impact of three different buffer amounts (for each of sodium bicarbonate and calcium carbonate) on simulated stomach acid (as measured by pH). To initiate the experiment, 2.0 g, 1.84 g or 1.6 g of sodium bicarbonate or 2.0 g, 1.84 g or 1.6 g calcium carbonate was individually added to a stomach beaker containing 100 ml of 0.1 N HCL; substantially simultaneously, a pH measurement was taken and the perfusion pumps were started to simulate stomach emptying. The 0.1 N HCl was added to the stomach beaker at a rate of 1.66 ml/minute while the solution in the stomach beaker was removed at the same flow rate; a constant volume was thereby maintained in the stomach beaker. Stomach beaker pH was measured at 10 second intervals over a period of approximately 60 minutes. Results are shown in FIG. 6 (sodium bicarbonate) and FIG. 7 (calcium carbonate).

Both of the buffers tested, when initially present in the stomach beaker in an amount of 1.84 g or 2.0 g, was able to maintain pH of the fluid in the stomach beaker above 5 for at least about 50 minutes. By contrast, when buffer was initially present in the stomach beaker in an amount of only 1.6 g, pH of the stomach beaker fluid was maintained above 5 for a period of less than 40 minutes.

For all formulations herein, multiple doses may be proportionally compounded as is known in the art. The invention has been described in an illustrative manner, and it is to be understood the terminology used is intended to be in the nature of description rather than of limitation. All patents and other references cited herein are incorporated herein by reference in their entirety. Many modifications, equivalents, and variations of the present invention are possible in light of the above teachings. Therefore, it is to be understood that within the scope of the appended claims, the invention may be practiced other than as specifically described.

1. An orally deliverable pharmaceutical composition comprising: (a) a therapeutically effective amount of at least one acid labile, substituted benzimidazole H⁺,K⁺-ATPase proton pump inhibitor; and (b) at least one buffering agent in a total amount greater than 10 mEq; wherein:
(i) the composition comprises substantially no poly[phosphoryl/sulfon]-ated carbohydrate; and
(ii) the composition is in the form of a solid, finished dosage unit.

2. The composition of claim 1 wherein the at least one buffering agent comprises substantially no amino acid buffering agent.

3. The composition of claim 1 wherein the at least one proton pump inhibitor is selected from the group consisting of omeprazole, lansoprazole, rabeprazole, esomeprazole, pantoprazole, pariprazole, leminoprazole, tenatoprazole, nepaprazole or an enantiomer, isomer, tautomer, ester, amide, derivative, prodrug, free base, or salt thereof.

4. The composition of claim 1 wherein the at least one proton pump inhibitor is present in the composition in a total amount of about 1 mg to about 1000 mg.

5. The composition of claim 1 wherein the at least one proton pump inhibitor is present in the composition in a total amount of about 10 mg to about 100 mg.

6. The composition of claim 1 wherein the at least one proton pump inhibitor is omeprazole, lansoprazole, or esomeprazole, or an enantiomer, isomer, tautomer, ester, amide, derivative, prodrug, free base, or salt thereof.

7. The composition of claim 1 wherein the at least one buffering agent is selected from the group consisting of sodium bicarbonate, potassium bicarbonate, magnesium hydroxide, magnesium lactate, magnesium gluconate, magnesium oxide, magnesium aluminate, magnesium carbonate, magnesium silicate, magnesium citrate, aluminum hydroxide, aluminum hydroxide/magnesium carbonate, potassium carbonate, potassium citrate, aluminum hydroxide/sodium bicarbonate coprecipitate, aluminum magnesium hydroxide, sodium citrate, sodium tartrate, sodium acetate, sodium carbonate, sodium polyphosphate, potassium polyphosphate, sodium pyrophosphate, potassium pyrophosphate, disodium hydrogenphosphate, dipotassium hydrogenphosphate, trisodium phosphate, tripotassium phosphate, potassium metaphosphate, calcium acetate, calcium glycerophosphate, calcium hydroxide, calcium lactate, calcium carbonate, calcium gluconate, calcium bicarbonate, calcium citrate, potassium phosphate, sodium phosphate, and mixtures thereof.

8. The composition of claim 1 wherein the at least one buffering agent is selected from the group consisting of sodium bicarbonate, sodium carbonate, calcium carbonate, magnesium oxide, magnesium hydroxide, magnesium carbonate, aluminum hydroxide, and mixtures thereof.

9. The composition of claim 1 wherein the at least one buffering agent is present in the composition in a total amount of about 10 mEq to about 150 mEq.

10. The composition of claim 1 wherein the at least one buffering agent is present in the composition in a total amount of about 10 mEq to about 50 mEq.

11. The composition of claim 1 wherein the at least one buffering agent is present in the composition in a total amount greater than 800 mg.

12. The composition of claim 1 wherein the at least one buffering agent is present in the composition in a total amount of at least about 920 mg.

13. The composition of claim 1 wherein the at least one buffering agent is present in the composition in a total amount of at least about 1000 mg.

14. The composition of claim 1 wherein the at least one buffering agent is present in the composition in a total amount of at least about 11 mEq.

15. The composition of claim 1 further comprising at least one pharmaceutically acceptable excipient selected from the group consisting of a carrier, a binder, a flavoring agent, a sweetening agent, a disintegrant, a flow aid, a lubricant, an adjuvant, a colorant, a diluent, a moistening agent, a preservative, a parietal cell activator, an anti-foaming agent, an antioxidant, a chelating agent, an antifungal agent, an antibacterial agent, and mixtures thereof.

16. The composition of claim 1 wherein the at least one proton pump inhibitor is present in the composition in a total amount of about 20 to about 40 mg and the at least one buffering agent is present in the composition in a total amount greater than 800 mg.

17. The composition of claim 1 wherein the at least one proton pump inhibitor is present in the composition in a total amount of about 20 to about 40 mg and the at least one buffering agent is present in the composition in a total amount of at least about 920 mg.

18. The composition of claim 1 wherein the at least one proton pump inhibitor is present in the composition in a total amount of about 20 to about 40 mg and the at least one buffering agent is present in the composition in a total amount of at least about 1000 mg.

19. The composition of claim 1 wherein the solid dosage form is selected from the group consisting of a tablet, a chewable tablet, a caplet, a capsule, a suspension tablet, a troche, a lozenge, and a powder.

20. The composition of claim 1 wherein the solid dosage form is a chewable tablet.

21. The composition of claim 1 wherein the solid dosage form is a capsule.

22. The composition of claim 1 wherein the at least one buffering agent is a single buffering agent.

23. An orally deliverable pharmaceutical composition comprising: (a) a therapeutically effective amount of at least one acid labile, substituted benzimidazole H⁺,K⁺-ATPase proton pump inhibitor; and (b) a combination of at least two non-amino acid buffering agents; wherein:
(i) the combination of at least two non-amino acid buffering agents comprises substantially no aluminum hydroxide-sodium bicarbonate co-precipitate;
(ii) the composition comprises substantially no poly[phosphoryl/sulfon]-ated carbohydrate; and
(iii) the composition is in the form of a solid dosage unit.

24. The composition of claim 23 wherein the at least one proton pump inhibitor is selected from the group consisting of omeprazole, lansoprazole, rabeprazole, esomeprazole, pantoprazole, pariprazole, leminoprazole, tenatoprazole, nepaprazole or an enantiomer, isomer, tautomer, ester, amide, derivative, prodrug, free base, or salt thereof.

25. The composition of claim 23 wherein the at least one proton pump inhibitor is present in the composition in a total amount of about 10 mg to about 100 mg.

26. The composition of claim 23 wherein the at least one proton pump inhibitor is omeprazole, lansoprazole, or esomeprazole, or an enantiomer, isomer, tautomer, ester, amide, derivative, prodrug, free base, or salt thereof.

27. The composition of claim 23 wherein the at least two non-amino acid buffering agents are selected from the group consisting of sodium bicarbonate, potassium bicarbonate, magnesium hydroxide, magnesium lactate, magnesium gluconate, magnesium oxide, magnesium aluminate, magnesium carbonate, magnesium silicate, magnesium citrate, aluminum hydroxide, aluminum hydroxide/magnesium carbonate, potassium carbonate, potassium citrate, aluminum hydroxide/sodium bicarbonate coprecipitate, aluminum magnesium hydroxide, sodium citrate, sodium tartrate, sodium acetate, sodium carbonate, sodium polyphosphate, potassium polyphosphate, sodium pyrophosphate, potassium pyrophosphate, disodium hydrogenphosphate, dipotassium hydrogenphosphate, trisodium phosphate, tripotassium phosphate, potassium metaphosphate, calcium acetate, calcium glycerophosphate, calcium hydroxide, calcium lactate, calcium carbonate, calcium gluconate, calcium bicarbonate, calcium citrate, potassium phosphate, sodium phosphate, and mixtures thereof.

28. The composition of claim 23 wherein the at least two non-amino acid buffering agents are selected from the group consisting of sodium bicarbonate, sodium carbonate, calcium carbonate, magnesium oxide, magnesium hydroxide, magnesium carbonate, aluminum hydroxide, and mixtures thereof.

29. The composition of claim 23 wherein the at least two non-amino acid buffering agents are present in the composition in a total amount of about 10 mEq to about 50 mEq.

30. The composition of claim 23 wherein the at least two non-amino acid buffering agents are present in the composition in a total amount greater than 800 mg.

31. The composition of claim 23 wherein the at least two non-amino acid buffering agents are present in the composition in a total amount of at least about 920 mg.

32. The composition of claim 23 wherein the at least two non-amino acid buffering agents are present in the composition in a total amount of at least about 1000 mg.

33. The composition of claim 23 wherein the at least two non-amino acid buffering agents are present in the composition in a total amount of at least about 11 mEq.

34. The composition of claim 23 further comprising at least one pharmaceutically acceptable excipient selected from the group consisting of a carrier, a binder, a flavoring agent, a sweetening agent, a disintegrant, a flow aid, a lubricant, an adjuvant, a colorant, a diluent, a moistening agent, a preservative, a parietal cell activator, an anti-foaming agent, an antioxidant, a chelating agent, an antifungal agent, an antibacterial agent, and mixtures thereof.

35. The composition of claim 23 wherein the at least one proton pump inhibitor is present in the composition in a total amount of about 20 to about 40 mg and the at least one buffering agent is present in the composition in a total amount greater than 800 mg.

36. The composition of claim 23 wherein the at least one proton pump inhibitor is present in the composition in a total amount of about 20 to about 40 mg and the at least one buffering agent is present in the composition in a total amount of at least about 920 mg.

37. The composition of claim 23 wherein the at least one proton pump inhibitor is present in the composition in a total amount of about 20 to about 40 mg and the at least one buffering agent is present in the composition in a total amount of at least about 1000 mg.

38. The composition of claim 23 wherein the solid dosage form is selected from the group consisting of a tablet, a chewable tablet, a caplet, a capsule, a suspension tablet, a troche, a lozenge, and a powder.

39. The composition of claim 23 wherein the solid dosage form is a chewable tablet.

40. The composition of claim 23 wherein the solid dosage form is a capsule.

41. An orally deliverable pharmaceutical composition comprising: (a) a therapeutically effective amount of at least one acid labile, substituted benzimidazole H⁺,K⁺-ATPase proton pump inhibitor in a total amount of about 10 to about 40 mg; and
(b) at least one non-amino acid buffering agent; wherein:
(i) the at least one non-amino acid buffering agent is present in the composition in a total amount greater than 800 mg;
(ii) the composition comprises substantially no poly[phosphoryI/sulfon]-ated carbohydrate; and
(iii) the composition is in the form of a solid dosage unit.

42. The composition of claim 41 wherein the at least one proton pump inhibitor is selected from the group consisting of omeprazole, lansoprazole, rabeprazole, esomeprazole, pantoprazole, pariprazole, leminoprazole, tenatoprazole, nepaprazole or an enantiomer, isomer, tautomer, ester, amide, derivative, prodrug, free base, or salt thereof.

43. The composition of claim 41 wherein the at least one proton pump inhibitor is present in the composition in a total amount of about 10 mg to about 100 mg.

44. The composition of claim 41 wherein the at least one proton pump inhibitor is omeprazole, lansoprazole, or esomeprazole, or an enantiomer, isomer, tautomer, ester, amide, derivative, prodrug, free base, or salt thereof.

45. The composition of claim 41 wherein the at least one non-amino acid buffering agent is selected from the group consisting of sodium bicarbonate, potassium bicarbonate, magnesium hydroxide, magnesium lactate, magnesium gluconate, magnesium oxide, magnesium aluminate, magnesium carbonate, magnesium silicate, magnesium citrate, aluminum hydroxide, aluminum hydroxide/magnesium carbonate, potassium carbonate, potassium citrate, aluminum hydroxide/sodium bicarbonate coprecipitate, aluminum magnesium hydroxide, sodium citrate, sodium tartrate, sodium acetate, sodium carbonate, sodium polyphosphate, potassium polyphosphate, sodium pyrophosphate, potassium pyrophosphate, disodium hydrogenphosphate, dipotassium hydrogenphosphate, trisodium phosphate, tripotassium phosphate, potassium metaphosphate, calcium acetate, calcium glycerophosphate, calcium hydroxide, calcium lactate, calcium carbonate, calcium gluconate, calcium bicarbonate, calcium citrate, potassium phosphate, sodium phosphate, and mixtures thereof.

46. The composition of claim 41 wherein the at least one non-amino acid buffering agent is selected from the group consisting of sodium bicarbonate, sodium carbonate, calcium carbonate, magnesium oxide, magnesium hydroxide, magnesium carbonate, aluminum hydroxide, and mixtures thereof.

47. The composition of claim 41 wherein the at least one non-amino acid buffering agent is present in the composition in a total amount of about 10 mEq to about 50 mEq.

48. The composition of claim 41 wherein the at least one non-amino acid buffering agent is present in the composition in a total amount of at least about 920 mg.

49. The composition of claim 41 wherein the at least one non-amino acid buffering agent is present in a total amount of at least about 11 mEq.

50. The composition of claim 41 further comprising at least one pharmaceutically acceptable excipient selected from the group consisting of a carrier, a binder, a flavoring agent, a sweetening agent, a disintegrant, a flow aid, a lubricant, an adjuvant, a colorant, a diluent, a moistening agent, a preservative, a parietal cell activator, an anti-foaming agent, an antioxidant, a chelating agent, an antifungal agent, an antibacterial agent, and mixtures thereof.

51. The composition of claim 41 wherein the solid dosage form is selected from the group consisting of a tablet, a chewable tablet, a caplet, a capsule, a suspension tablet, a troche, a lozenge, and a powder.

52. The composition of claim 41 wherein the solid dosage form is a chewable tablet.

53. The composition of claim 41 wherein the solid dosage form is a capsule.

54. An orally deliverable pharmaceutical composition comprising: (a) a therapeutically effective amount of at least one acid labile, substituted benzimidazole H⁺,K⁺-ATPase proton pump inhibitor; and (b) at least one buffering agent in a total amount greater than 10 mEq; wherein:
(i) the composition comprises substantially no poly[phosphoryl/sulfon]-ated carbohydrate;
(ii) the composition is in the form of a solid dosage unit; and
(iii) if an amino acid buffering agent is present, at least one of the following conditions is met:
(1) the weight ratio of amino acid buffering agent:proton pump inhibitor is greater than 20:1;
(2) the composition comprises at least two non-amino acid buffering agents;
(3) the composition comprises at least one non-amino acid buffering agent wherein the weight ratio of the at least one non-amino acid buffering agent:proton pump inhibitor is greater than 20: 1; and/or
(4) the weight ratio of total buffering agent:proton pump inhibitor is greater than 40:1.

55. A method of treating an acid related gastrointestinal disorder in a subject in need thereof, the method comprising orally delivering to the subject a composition comprising: (a) a therapeutically effective amount of at least one acid labile, substituted benzimidazole H⁺,K⁺-ATPase proton pump inhibitor; and (b) at least one buffering agent in a total amount greater than 10 mEq; wherein:
(i) the composition comprises substantially no poly[phosphoryl/sulfon]-ated carbohydrate; and
(ii) the composition is in the form of a solid, finished dosage unit.
